(19) United States
(12) Patent Application Publication
(10) Pub. No.: US 2026/0048075 A1
(43) Pub. Date: Feb. 19, 2026

(54) Inhibition of TCF4/ITF2 in the Treatment of Cancer

(71) Applicants: VIB VZW, Gent (BE); Katholieke Universiteit Leuven, Leuven (BE)

(72) Inventors: Jean-Christophe Marine, Leuven (BE); Florian Rambow, Leuven (BE); Joanna Pozniak, Leuven (BE); Dennis Pedri, Leuven (BE)

(21) Appl. No.: 19/102,126

(22) PCT Filed: Aug. 8, 2023
(86) PCT No.: PCT/EP2023/071976

§ 371 Date: Feb. 7, 2025

(30) Foreign Application Priority Data
Aug. 10, 2022 (EP) 22189705.1

(51) Int. Cl. A61K 31/7105; A61K 45/00; A61P 35/00; C12N 15/113; C12Q 1/6886; G01N 33/574

(52) U.S. Cl. CPC A61K 31/7105; A61K 45/05; A61P 35/00; C12N 15/113; C12Q 1/6886; G01N 33/57492; C12N 2310/14; C12N 2310/531; C12Q 2600/106; C12Q 2600/158

(57) ABSTRACT

The invention relates to the application of inhibition of transcription factor 4 (TCF4/ITF2) in the treatment of cancer. This in particular in combination therapy in conjunction with an immunotherapeutic compound (such as for treating cancers poorly responding or refractive to immunotherapy). In particular, inhibition of TCF4/ITF2 is capable of restoring response to immunotherapy such as immune checkpoint inhibitor therapy. Expression levels of TCF4/ITF2 as well as levels of mesenchymal-like cancer cells in a cancer lesion are predictive of future response to immunotherapy early after initiation of the immunotherapy. Further part of the invention are methods of detecting mesenchymal-like cancer cells.

### CROSS REFERENCE TO RELATED APPLICATIONS

This application is a national phase entry under 35 U.S.C. § 371 of International Patent Application PCT/EP2023/071976, filed Aug. 8, 2023, designating the United States of America and published in English as International Patent Publication WO2024/033381 on Feb. 15, 2024, which claims the benefit under Article 8 of the Patent Cooperation Treaty to European Patent Application Serial No. 22189705.1, filed Aug. 10, 2022, the entireties of which are hereby incorporated by reference.

### INCORPORATION BY REFERENCE

The ST.26 XML Sequence listing named “10488-10882-Sequence Listing ST26”, created on Aug. 8, 2023, and having a size of 8,192 bytes, is hereby incorporated herein by this reference in its entirety.

### FIELD OF THE INVENTION

The invention relates to the application of inhibition of transcription factor 4 (TCF4/ITF2) in the treatment of cancer. This in particular in combination therapy in conjunction with an immunotherapeutic compound (such as for treating cancers poorly responding or refractive to immunotherapy). In particular, inhibition of TCF4/ITF2 is capable of restoring response to immunotherapy such as immune checkpoint inhibitor therapy. Expression levels of TCF4/ITF2 as well as levels of mesenchymal-like cancer cells in a cancer lesion are predictive of future response to immunotherapy early after initiation of the immunotherapy. Further part of the invention are methods of detecting mesenchymal-like cancer cells.

### BACKGROUND OF THE INVENTION

Despite several breakthroughs in the field, metastatic melanoma (MM) continues to be a major clinical challenge (Larkin et al. 2019, N Engl J Med 381:1535-1546; Robert et al. 2019, N Engl J Med 381:626-636). Although treatment outcomes have substantially improved since the introduction of immune checkpoint blockade (ICB) (Wolchok et al. 2017, N Engl J Med 377:1345-1356; Robert et al. 2019, Lancet Oncol 20:1239-1251) approximately half of the MM patients fail to gain any durable survival benefit. One of the key challenges is therefore to elucidate why ICB therapies, such as anti-PD-1, anti-CTLA-4 or their combination, are effective in some, but not all, patients, and ultimately identify rational therapeutic (combination) strategies that overcome resistance.

In general, tumor-extrinsic and intrinsic mechanisms can drive resistance to ICB (Kalbasi et al. 2020, Nat Rev Immunol 20, 25-39; Liu et al. 2019, Nat Med 25:1916-1927). Tumor mutational burden has, for instance, been associated to ICB response, bolstering immunogenicity through increased neoantigen formation (Schumacher & Schreiber 2015, Science 348:69-74; Van Allen et al. 2015, Science 350:207-211). Mutations in genes encoding components of the antigen processing and/or presentation (APP) machinery (e.g., MHC class I, B2-microglobulin) can lead to ICB resistance. Similarly, tumors with inactivating mutations in JAK1/JAK2, are associated with loss of interferon responsiveness, and thereby with resistance to PD-1 blockade (Shin et al. 2017, Cancer Discov 7:188-201; Zaretsky et al. 2016, N Engl J Med 375:819-829). In addition, there is increasing evidence that melanoma cells can adopt a variety of phenotypic states through non-genetic reprogramming, and thereby exhibit different sensitivities to cancer treatments, including ICB (Rambow et al. 2019, Genes Dev 33:1295-1318). For instance, a subset of melanomas harbor tumor cells capable of expressing MHC class II (HLA-DR) molecules was described and their presence is associated with a CD8+ tumor infiltrate and favorable response to anti-PD1 therapy (Johnson et al. 2016, Nat Commun 7:10582).

Dedifferentiation of melanoma cells was previously described as another nongenetic mechanism that drives immune escape and resistance to adoptive T cell transfer (Landsberg et al. 2012, Nature 490:412-416; Mehta et al. 2018, Cancer Discov 8:935-943). Based on bulk RNA-seq data analyses of anti-PD-1 treated melanoma samples, collected at baseline and upon progression, it was further proposed that dedifferentiation may also be a mechanism driving resistance to ICB (Hugo et al. 2017, Cell 168:542). Deconvolution of additional bulk RNA-seq datasets and immunostaining further confirmed the enrichment of a dedifferentiated (NGFR^{high}) Neural-Crest-like program in tumors associated with immune-exclusion (Boshuizen et al. 2020, Nat Commun 11:3946) and resistance to immunotherapy (Liu et al. 2021, Nat Med 27:985-992). Mechanistically, dedifferentiation was proposed to dampen response to ICB due to a decrease in expression and/or presentation of melanocytic antigens (Landsberg et al. 2012, Nature 490:412-416; Mehta et al. 2018, Cancer Discov 8:935-943), MHC class I downregulation (Lee et al. 2020, Nat Commun 11:1897), and secretion of the neurotrophic factor BDNF, which contributes to resistance to antigen-specific T cells (Boshuizen et al. 2020, Nat Commun 11:3946). Consistent with these findings, the innate PD-1 inhibitor resistance (IPRES) signature (which was defined based on the analysis of bulk RNA-seq data and includes 26 gene signatures associated with dedifferentiation) was associated with poor response to PD-1 inhibitor in pre-treatment biopsies (Hugo et al. 2017, Cell 168:542). However, such an association could not be established in other melanoma cohorts (Riaz et al. 2017, Cell 171:934-949.e16; Chen et al. 2016, Cancer Discov 6:827-837). The difficulty in identifying reliable predictive information at baseline using bulk transcriptomic data was further highlighted by the lack of reproducibility when predicting response to ICB using IMPRES (Auslander et al. 2018, Nat Med 24:1545-1549), yet another gene expression signature derived from bulk RNA-seq datasets (Lee et al. 2020, Nat Commun 11:1897; Carter et al. 2019, Nat Med 25:1833-1835). Bulk longitudinal analyses later confirmed that a robust pre-treatment biomarker is unlikely to capture the heterogenous nature of cancer and/or anticipate the rapid evolution of tumor phenotypes under ICB therapy (Lee et al. 2020, Nat Commun 11:1897).

It has therefore become clear that understanding resistance to ICB requires single-cell resolution and temporal dissection of the entire cellular architecture of the tumor/cancer ecosystem. Using scRNA-seq, a MYC-driven malignant gene expression signature associated with immune evasion and T-cell exclusion was recently identified (Jerby-Arnon et al. 2018, Cell 175:984-997.e24). Although very informative, this study was limited by the recovery of a relatively small number of malignant cells and absence of patient-matched samples across both time points. In addition, only one responder was identified in the discovery cohort. It is important to note that like in the previous study by the same group (Tirosh et al. 2016, Science 352:189-196), most biopsies in this study originated from patients with prior exposure to diverse treatments. Therefore, a comprehensive and dynamic view of the cellular architecture and of the drug-naïve melanoma transcriptomic landscape during ICB therapy is still lacking.

The basic helix-loop-helix (bHLH) transcription factor 4 (TCF4, also known as ITF2) has been associated with tumor suppressive function as established for multiple cancer types (e.g. Hellwig et al. 2019, Acta Neuropathol 137:657-673; Shin et al. 2014, Gastroenterology 147:430-442; Grill et al. 2015, Biochem Biophys Res Commun 461:249-253). In breast cancer, loss of TCF4/ITF2 conferred resistance to chemotherapy (Ruiz de Garibay et al. 2018, Dis Model Mech 11: dmm032292) whereas in melanoma, loss of TCF4/ITF2 was associated with enhanced susceptibility to selumetinib (an inhibitor of MEK) (Hur et al. 2017, Oncotarget 8:41387-41450).

Overexpression of transcription factor 4 (TCF4/ITF2) has been linked, via influencing miR-125b expression, to progression of melanoma invasion (Rambow et al. 2016, J Invest Dermatol 136:1229-1237). Furthermore, TCF4/ITF2 has been identified as one of the many redundant transcription factors regulating epithelial-to-mesenchymal transition (EMT) whereas TCF4/ITF2 overexpressing cells showed no or very low tumorigenic capacity but displayed increased invasive behavior. As such, TCF4/ITF2 may be more important in establishment of metastases than in establishment of a primary tumor (Sobrado et al. 2009, J Cell Sci 122:1014-1024; Cano & Portillo 2010, Cell Adh Migr 4:56-60).

Although in general there appears to be a relation between EMT and immune evasion, specific targets/mechanisms have not been singled out (Wang et al. 2021, Precision Oncology 5:56; Terry et al. 2017, Mol Oncol 11:824-846). EMT in itself has been suggested as therapeutic target although this, apart from the complexity (several EMT transcription factors have complementary and redundant functions), appears associated with a number of risks and problems (Jonckheere et al. 2021, Cells Tissues Organs DOI-10.1159-000512218).

Importantly, TCF4/ITF2 should not be confused with the immune regulator transcription factor 7-like 2 (TCF7L2, a member of the TCF/LEF family of transcription factors) which is confusingly also referred to as TCF4 (T-cell factor 4) (Cano & Portillo 2010, Cell Adh Migr 4:56-60). For instance, Cen et al. 2021 (Oncogene 40:5984-5992) appears to relate to TCF4 but in fact discloses work on TCF7L2.

### SUMMARY OF THE INVENTION

In one aspect, the invention relates to an inhibitor of transcription factor 4 (TCF4/ITF2) for use in treating or inhibiting cancer, or for use in inhibiting progression, relapse or metastasis of cancer, wherein the cancer is poorly responding to or resistant to immune checkpoint inhibitor therapy or to therapy comprising an immune checkpoint inhibitor.

A further aspect relates to an inhibitor of transcription factor 4 (TCF4/ITF2) for use in treating or inhibiting cancer, or for use in inhibiting progression, relapse or metastasis of cancer,
wherein the patient having cancer has received at least one administration of an immune checkpoint inhibitor therapy or of a therapy comprising an immune checkpoint inhibitor; and
wherein, after the at least one administration of an immune checkpoint inhibitor therapy or of a therapy comprising an immune checkpoint inhibitor the cancer is expressing levels of TCF4/ITF2 corresponding to TCF4/ITF2 expression levels in the same type of cancer of patients known not to respond to the immune checkpoint inhibitor therapy or to the therapy comprising an immune checkpoint inhibitor; and/or
wherein the patient having cancer has received at least one administration of an immune checkpoint inhibitor therapy or of a therapy comprising an immune checkpoint inhibitor; and
wherein, after the at least one administration of an immune checkpoint inhibitor therapy or of a therapy comprising an immune checkpoint inhibitor the cancer is comprising a number of mesenchymal-like cells corresponding to numbers of mesenchymal-like cells in the same type of cancer of patients known not to respond to the immune checkpoint inhibitor therapy or to the therapy comprising an immune checkpoint inhibitor.

In one embodiment, the inhibitor of TCF4/ITF2 is for use according to the above in combination with immune checkpoint inhibitor therapy or with a therapy comprising an immune checkpoint inhibitor.

In a further embodiment, the inhibitor of TCF4/ITF2 is a specific inhibitor of TCF4/ITF2, and is selected from a DNA nuclease specifically knocking out or disrupting TCF4/ITF2, an RNase specifically targeting TCF4/ITF2, or an inhibitory oligonucleotide specifically targeting TCF4/ITF2.

A further aspect of the invention relates to an immune checkpoint inhibitor for use in treating or inhibiting cancer, or for use in inhibiting progression, relapse or metastasis of cancer, in combination with an inhibitor of transcription factor 4 (TCF4/ITF2). In one embodiment thereto the inhibitor of TCF4/ITF2 is a specific inhibitor of TCF4/ITF2.

The invention further relates to combinations of an inhibitor of transcription factor 4 (TCF4/ITF2) and an immune checkpoint inhibitor, and to compositions comprising such a combination. Such combinations or compositions in particular are for use as a medicine.

The invention relates in another aspect to methods for determining or predicting response of a cancer patient to immune checkpoint inhibitor therapy or to therapy comprising an immune checkpoint inhibitor, comprising:
determining, assessing, measuring, or quantifying the level of TCF4/ITF2 expression in a sample obtained from a cancer lesion, wherein the sample is obtained after the patient having cancer received at least one administration of an immune checkpoint inhibitor therapy or of a therapy comprising an immune checkpoint inhibitor;
determining or predicting the cancer patient having cancer to respond to the immune checkpoint inhibitor therapy or therapy comprising the immune checkpoint inhibitor when the TCF4/ITF2 expression level in the sample corresponds to TCF4/ITF2 expression levels in the same type of cancer of patients known to respond to the immune checkpoint inhibitor therapy or to the therapy comprising an immune checkpoint inhibitor.

The invention relates in another aspect to methods for determining or predicting response of a cancer patient to immune checkpoint inhibitor therapy or to therapy comprising an immune checkpoint inhibitor, comprising:
determining, assessing, measuring, or quantifying the number of mesenchymal-like cancer cells in a sample obtained from a cancer lesion, wherein the sample is obtained after the patient having cancer received at least one administration of an immune checkpoint inhibitor therapy or of a therapy comprising an immune checkpoint inhibitor;
determining or predicting the cancer patient having cancer to respond to the immune checkpoint inhibitor therapy or therapy comprising the immune checkpoint inhibitor when the number of mesenchymal-like cancer cells in the sample corresponds to the number of mesenchymal-like cancer cells in the same type of cancer of patients known to respond to the immune checkpoint inhibitor therapy or to the therapy comprising an immune checkpoint inhibitor.

Such methods for determining or predicting response of a cancer patient to immune checkpoint inhibitor therapy or to therapy comprising an immune checkpoint inhibitor can optionally further comprise:
determining, assessing, measuring, or quantifying the number of antigen-presenting cancer cells in a sample obtained from a cancer lesion, wherein the sample is obtained before the patient having cancer received at least one administration of an immune checkpoint inhibitor therapy or of a therapy comprising an immune checkpoint inhibitor;
determining or predicting the cancer patient having cancer to respond to the immune checkpoint inhibitor therapy or therapy comprising the an immune checkpoint inhibitor when the number of antigen-presenting cancer cells in the sample corresponds to the number of antigen-presenting cancer cells in the same type of cancer of patients known to respond to the immune checkpoint inhibitor therapy or to the therapy comprising an immune checkpoint inhibitor.

The invention relates in another aspect to methods for determining the presence of mesenchymal-like cancer cells in a tumor, comprising:
assessing the presence of a protein and/or RNA encoding the protein in a sample obtained from the tumor, wherein the protein and/or RNA is a product of a gene of Table 1 or 3;
assessing the presence of a protein and/or RNA encoding the protein in a sample obtained from the tumor, wherein the protein and/or RNA is a product of a gene of Table 4 or 5;
assessing the presence of a protein and/or RNA encoding the protein in a sample obtained from the tumor, wherein the protein and/or RNA is a product of a cancer cell gene;
determining mesenchymal-like cancer cells to be present in a tumor when detecting cancer cells in which a product of a gene of Table X1 or X2, a product of a gene of Table Y, and a product of a cancer cell gene are present.

Alternatively, such methods are comprising detecting co-localization of a product of a gene of Table 1 or 3, a product of a gene of Table 4 or 5, and a product of a cancer cell gene in the same cancer cell in a sample obtained from a patient having cancer, and designating such cancer cell as mesenchymal-like cancer cell.

In one embodiment to these latter methods, the gene of Table 1 or 3 is THY1, LUM, DCN, and/or TCF; the gene of Table 4 or 5 is CDH19, S100A1, MITF and/or SOX10; and the cancer cell gene is MITF and/or SOX10, and/or is gene with a mutation specific to the cancer cell.

### BRIEF DESCRIPTION OF THE DRAWINGS

FIG. 1. Schematic representation of the sample collection and main experimental techniques used

FIG. 2. The human melanoma landscape
A: AUCell scores of the functionally enriched marker genes per mouse malignant cell state (acquired from Karras et. al, Nature, in press), averaged and plotted across the malignant cell states.
B: AUCell scores of the top 100 marker genes of each of the human malignant cell states averaged and replotted across the malignant cell states.

FIG. 3. Developing methods to detect Melanoma MES cells
A: Top 50 marker genes of the MES cell state derived from testing against all other malignant states, and plotted between the CAFs and the MES state (left). Minimal Lineage Genes signature (MLGs) derived from testing MES versus CAFs, and plotted between the CAFs and the MES state (right).
B: AUCell score of the top 50 marker genes of the Mesenchymal-like (MES) cell state plotted across all human malignant cell states.

FIG. 4. Identification of melanoma MES cells
A: Four selected marker genes of the Minimal Lineage Genes signature (MLG) (CDH19, S100A1, MITF, SOX10) were plotted between the CAFs and the Mesenchymal-like state; and four selected marker genes of the MES state (THY1, LUM, DCN, TCF4) were plotted between the CAFs and the Mesenchymal-like state.
B: Selected marker genes of FIG. 4A genes plotted across all human malignant cell states.

FIG. 5. In situ mapping of melanoma MES cells
Combined mIHC and multiplex FISH image of a representative treatment naïve lymph node metastasis CD45, CD31 and TCF4 protein stains are shown, whereas FISH of the four selected genes for the MLGs ((MITF, SOX10, S100A1 and CDH19) and MES state (DCN, TCF4, THY1 and LUM) are combined (top). A split of each four genes is shown. Nuclei of Mesenchymal-like cells co-expressing MLGs and MES genes are segmented.

FIG. 6. Antigen Presentation- and MES-cell states are associated with response to immunotherapy Percentage of Antigen Presentation and Mesenchymal-like cell states out of total malignant cells per sample compared between Responder (R) and Non-Responder (NR) to immune checkpoint blockade therapy at both timepoints (two-sided Wilcoxon test). BT/R: before treatment/responder; OT/R: on treatment/responder; BT/NR: before treatment/non-responder; OT/NR: on treatment/non-responder.

FIG. 7. TCF4 orchestrates multiple melanoma transcriptional programs
Heatmap representing expression of gene sets related to antigen presentation, IFN signaling, and EMT without and with silencing of TCF4 in MM099 cells (bulk RNA-seq, n=2 biological replicates). siCTRL: irrelevant siRNA not targeting TCF4: siTCF4: siRNA specifically targeting TCF4. “Hot” fields (high (er) gene expression; above zero) are marked with an asterisk. “Cold” fields (low (er) gene expression; below zero) are unmarked.

FIG. 8. TCF4 silencing increases melanoma cell death
Normalized dead cell counts upon co-culture of MM099 cells with activated HLA-matched PBMCs upon silencing of TCF4 (siTCF4: siRNA specifically targeting TCF4), versus non-silencing of TCF4 (siCTRL: irrelevant siRNA not targeting TCF4) (n=3 biological replicates, paired t-test, ****p<0.0001). Line and filled area indicate mean±SEM, respectively (n=6 technical replicates). As negative controls, mono-cultured MM099 cells (activated HLA-matched PBMCs absent) were treated with siTCF4 or siCTRL.

FIG. 9. TCF4 targeting through BET-inhibition
Cell growth of MM029 cells upon treatment with BET inhibitor (ARV-771 300 nM), BRAF- and MEK-inhibitors (dabrafenib 50 nM, trametinib 10 nM), or a combination thereof. Error bars indicate mean±SEM (n=6 technical replicates, paired Student's t-test, ****p<0.0001). DMSO without inhibitor was used as negative control.

FIG. 10. Decreased activation of tumor-infiltrating lymphocytes (TILs) upon co-culture of TILs with TCF4 overexpressing melanoma cells
Overexpression of TCF4 in the melanoma Ma-Mel-86c cell line was obtained via a doxycycline-inducible TCF4 gene construct (CTRL: TCF4 overexpression not induced; TCF4 OE: overexpression of TCF4 induced by doxycycline). Activation of the TILs was measured with flow cytometry via determining TNFα- and IFNγ-positive TILs.

FIG. 11. Knockdown of TCF4 expression in melanoma cells increases T-cell activation independent of melanoma antigens
An immortalized Jurkat CD8−/−TCR−/−T cells transduced to stably express exclusively the TCR recognising the NY-ESO-1 A02 (1G4) peptide (NYE-TCR Jurkat cells) was co-cultured with A375 melanoma cells engineered to express the NY-ESO-1 A02 (1G4) peptide (NY-ESO-1 cells). Silencing of TCF4 in NY-ESO-1 cells led to a marked decrease in activation of the NYE-TCR Jurkat cells. NT: untreated; PMA+iono: phorbol 12-myristate 13-acetate and ionomycin (positive control activation Jurkat T cells in absence of antigen); CTRL: control (co-culture of NYE-TCR Jurkat cells with wild-type A375 melanoma transfected with a scramble siRNA (siCTRL) or with an siRNA targeting TCF4 (siTCF4); NY-ESO-1: co-culture of NYE-TCR Jurkat cells with NY-ESO-1 cells transfected with a scramble siRNA (siCTRL) or with an siRNA targeting TCF4 (siTCF4).

FIG. 12. Potentiation of immune checkpoint inhibitor activity by TCF4 knockdown
A. Tumor growth (indicated as volume in mm3) of control mice bearing tumors with a control knockdown (shRNA scramble) treated with either unrelated IgG control (“SCR_IgG”) or anti-PD1 immunotherapy (“SCR_aPD1”) compared to mice bearing tumors with a knockdown for TCF4 (shRNA TCF4) treated with either unrelated IgG control (“TCF4_IgG”) or in combination with anti-PD1 immunotherapy (“TCF4_aPD1”). B. Proxy progression-free survival curves (days until tumor volume reaches 300 mm3) of control mice bearing tumors with a control knockdown (shRNA scramble) treated with either IgG control (“SCR_IgG”) or anti-PD1 immunotherapy (“SCR_aPD1”) compared to mice bearing tumors with a knockdown for TCF4 (shRNA TCF4) treated with either IgG control (“TCF4_IgG” or in combination with anti-PD1 immunotherapy (“TCF4_aPD1”).

### DETAILED DESCRIPTION

The clinical success of immune checkpoint blockade (ICB) in cancer treatment is limited to only a subset of patients. The work leading to the current invention describes an increasingly complex tumor transcriptional landscape, its evolution under ICB and identifies a biomarker of response and a therapeutic strategy to increase immunogenicity of ICB-refractory tumors.

To identify cancer cell-intrinsic mechanisms driving resistance, single-cell RNA-sequencing (scRNAseq) data from a prospective longitudinal cohort of melanoma patients on ICB therapy were generated. Integrating these data with murine scRNAseq datasets, a comprehensive view of the cellular architecture of the treatment-naive melanoma ecosystem was established, and 6 evolutionarily conserved melanoma transcriptional metaprograms (melanocytic or MEL, mesenchymal-like or MES, neural crest-like, antigen presentation, stress-hypoxia response and stress-p53 response) were defined. Spatial multi-omics revealed a non-random geographic distribution of cell states that is, at least partly, driven by the tumor microenvironment. Importantly, two of the metaprograms associated with divergent clinical responses to ICB. While the antigen presentation cell population was more abundant in tumors from patients who exhibited a clinical response to ICB, MES cells were significantly enriched in early on-treatment biopsies from non-responders and their presence significantly predicted lack of response. Critically, in addition to driving the MES transcriptional program, TCF4 actively suppressed both the melanocytic and antigen presentation programs. Expression of transcription factor 4 (TCF4/ITF2) has not been proposed as regulator of genes of the antigen processing/presentation program. This latter role favorably positions TCF4/ITF2 as an ideal target to improve the response to ICB in particular. Indeed, defective or improper (levels of) tumor antigen processing and/or presentation have been linked with resistance to immune checkpoint blockers (e.g. Jenkins et al. 2018, Br J Cancer 118:9-16).

In work leading to the current invention as explained in the Figures and Examples, it was demonstrated that expression of the transcription factor 4 (TCF4/ITF2) was upregulated, and the number of mesenchymal-like (MES) cancer cells was higher in cancer patients treated with but not clinically responding to immune checkpoint inhibitor therapy. This compared to TCF4/ITF2 expression levels and number of MES cancer cells in cancer patients treated with and clinically responding to immune checkpoint inhibitor therapy. In particular, the increased expression of TCF4/ITF2 and MES cancer cell numbers in non-responders vs responders was detected early during therapy and subsequently linked to clinical (absence of/non-) response to the therapy.

Furthermore, inhibition of expression of TCF4/ITF2 was shown both to downregulate expression of genes of the epithelial-mesenchymal transition (EMT) program and to upregulate expression of genes of the antigen presentation/processing program, therewith increasing visibility of cancer cells to the cancer patient's immune system, a unique combination of features leading to breaking the non-response of the cancer patient to the immune checkpoint inhibitor therapy.

Thus, TCF4/ITF2 both is an early marker of clinical non-response during treatment of a tumor or cancer with ICB, and is in such non-responding patients as well a therapeutic target for sensitizing these patients to ICB therapy. This is further corroborated by data indicating that TCF4/ITF2 silencing in MES cancer cells indeed is increasing T-cell-mediated death of the MES cancer cells.

Distinguishing MES cancer cells and cancer-associated fibroblasts (CAFs) has so far been impossible. The work leading to the current invention solved this problem by providing a set of markers capable of distinguishing MES cancer cells and CAFs. Such markers provide a further tool to effectively predict response of cancer patients to ICB therapy.

Therefore, in a first aspect, the invention relates to an inhibitor of transcription factor 4 (TCF4/ITF2) for use in treating or inhibiting cancer or a tumor, or for use in inhibiting progression, relapse or metastasis of cancer or of a tumor, wherein the cancer or tumor is poorly responding to, resistant to, or refractory to immunotherapy, or is poorly responding to, resistant to, or refractory to treatment or therapy comprising immunotherapy. Alternatively, the invention relates to use of an inhibitor of transcription factor 4 (TCF4/ITF2) in the manufacture of a medicine or medicament for treating or inhibiting cancer or a tumor, or for use in inhibiting progression, relapse or metastasis of cancer or a tumor, wherein the cancer or tumor is poorly responding to, resistant to, or refractory to immunotherapy, or is poorly responding to, resistant to, or refractory to treatment or therapy comprising immunotherapy. Further alternatively, the invention relates to methods of treating or inhibiting cancer or a tumor, or of inhibiting progression, relapse or metastasis of cancer or a tumor, in a subject or individual (in particular a mammalian subject or mammal, such as a human subject or human), such methods comprising administering an inhibitor of transcription factor 4 (TCF4/ITF2) to the subject or individual, and wherein the cancer or tumor is poorly responding to, resistant to, or refractory to immunotherapy, or is poorly responding to, resistant to, or refractory to treatment or therapy comprising immunotherapy. The administration of the TCF4/ITF2 inhibitor, such as a therapeutically effective amount of the TCF4/ITF2 inhibitor, to the subject or individual results in the treatment or inhibition of cancer or tumor growth, or in inhibition of the progression, relapse or metastasis of cancer or tumor growth.

In particular, the immunotherapy is a treatment or therapy with an immune checkpoint inhibitor, or a treatment or therapy comprising an immune checkpoint inhibitor. Poor response to, resistance to, or refractory to immunotherapy (such as checkpoint inhibitor therapy) is herewith understood as either non-response (NR) or partial response (PR) to the immunotherapy (such as immune checkpoint inhibitor therapy), in particular to a therapy consisting of administration of immunotherapy only, or in particular to a therapy comprising administration of an immunotherapeutic compound or agent. When such therapy is comprising administration of an immunotherapeutic compound or agent, it is in particular not comprising, or is excluding, administration of an inhibitor of TCF4/ITF2. The poor response, resistance, non-response or partial response in particular may be based on clinical experience or observation and/or may be based on analysis of biomarkers that are predictive or prognostic for efficacy of immune therapy (such as immune checkpoint inhibitor treatment or therapy). Such biomarkers may be analyzed in tumor tissue (e.g. tumor biopsy) or in a liquid biopsy taken e.g. from the patient's circulation (e.g. cfDNA, ctDNA, circulating cancer cells, exosomes, serum proteins . . . ). Novel such biomarkers as identified in the work leading to this invention are described hereinafter.

Cancer immunotherapy has provided patients with a promising treatment option. Therapeutic regimens such as adoptive T cell transfer (ACT), cancer vaccines and immune checkpoint inhibitors (e.g. anti-PD-1, anti-PDL1 or anti-CTLA-4 antibodies), harness the ability of the immune system to recognize and reject the tumor (Smyth et al. 2015, Nat Rev Clin Oncol 13:143-158). However, despite high response rates, with prolonged survival in a subset of melanoma (e.g. Schadendorf et al. 2015, J Clin Oncol 33:1889-189), lung (e.g. Borghaei et al. 2015, N Engl J Med 373:1627-1639), and renal cancer patients (e.g. Motzer et al. 2015, N Engl J Med 373:1803-1813), for several other tumors such as mismatch repair (MMR)-proficient colorectal cancer (CRC) (e.g. Le et al. 2015, N Engl J Med 372:1509-2520) and pancreatic ductal adenocarcinoma (PDAC) (e.g. Sarantis et al. 2020, World J Gastrointest Oncol 12:173-181) immunotherapy fails to show any clinical benefit.

In yet a further aspect, the invention relates to an inhibitor of transcription factor 4 (TCF4/ITF2) for use in treating or inhibiting cancer or for use in inhibiting progression, relapse or metastasis of cancer, in combination with immunotherapy; or wherein the treatment or inhibition further comprises therapy with or administration of an immunotherapy/immunotherapeutic compound or agent; or wherein the treatment or inhibition is combined with a therapy comprising an immunotherapy or with administration of an immunotherapy/immunotherapeutic compound or agent (to the subject or individual having the cancer or tumor).

Alternatively, the invention relates to use of an inhibitor of transcription factor 4 (TCF4/ITF2) in the manufacture of a medicament for use in combination with (administration of) an immunotherapy/immunotherapeutic compound or agent for treating or inhibiting cancer or for inhibiting progression, relapse or metastasis of cancer (in a subject or individual having the cancer or tumor); or wherein the treatment or inhibition further comprises therapy with or administration of an immunotherapy/immunotherapeutic compound or agent; or wherein the treatment or inhibition is combined with a therapy comprising an immunotherapy or with administration of an immunotherapy/immunotherapeutic compound or agent (to the subject or individual having the cancer or tumor).

Alternatively, the invention relates to use of an inhibitor of transcription factor 4 (TCF4/ITF2) in the manufacture of a medicament for treating or inhibiting cancer or for inhibiting progression, relapse or metastasis of cancer (in a subject or individual having the cancer) in combination with an immunotherapy (for treating or inhibiting cancer or for inhibiting progression, relapse or metastasis of cancer); or in combination with administering an immunotherapy/immunotherapeutic compound or agent to the subject or individual; or wherein the treatment or inhibition further comprises therapy with or administration of an immunotherapy/immunotherapeutic compound or agent; or wherein the treatment or inhibition is combined with a therapy comprising an immunotherapy or with administration of an immunotherapy/immunotherapeutic compound or agent (to the subject or individual having the cancer or tumor).

Yet a further alternative aspect of the invention relates to an inhibitor of transcription factor 4 (TCF4/ITF2) for use in increasing antigen presentation by tumor cells; to use of an inhibitor of TCF4/ITF2 in the manufacture of a medicament for increasing antigen presentation by tumor cells; or to methods of increasing the presentation of antigens by tumor cells including administering an inhibitor of TCF4/ITF2 to a subject having cancer or a tumor.

Another aspect of the invention relates to an inhibitor of transcription factor 4 (TCF4/ITF2) for use in (a method of/the manufacture of a medicament for) treating or inhibiting cancer, or for use in (a method of/the manufacture of a medicament for) inhibiting progression, relapse or metastasis of cancer,
wherein the patient having cancer has received at least one administration of immunotherapy/immunotherapeutic compound; and
wherein, after the at least one administration of the immunotherapy/immunotherapeutic compound the cancer is expressing levels of TCF4/ITF2 corresponding to TCF4/ITF2 expression levels in the same type of cancer of patients known not to respond (clinically) to the immunotherapy/immunotherapeutic compound; and/or:
wherein the patient having cancer has received at least one administration of immunotherapy/immunotherapeutic compound; and wherein, after the at least one administration of an immunotherapy/immunotherapeutic compound the cancer is comprising a number of mesenchymal-like cells corresponding to numbers of mesenchymal-like cells in the same type of cancer of patients known not to respond (clinically) to the immunotherapy/immunotherapeutic compound.

When TCF4/ITF2 expression levels in the tumor or cancer cells of the patients having the tumor or cancer corresponds to clinical non-response to the immunotherapy/immunotherapeutic compound, then the inhibitor of TCF4/ITF2 is administered to the patient having cancer. Alternatively, when the number of mesenchymal-like cells in the tumor or cancer cells of the patients having the tumor or cancer corresponds to clinical non-response to the immunotherapy/immunotherapeutic compound, then the inhibitor of TCF4/ITF2 is administered to the patient having cancer. Further alternatively, when TCF4/ITF2 expression levels and number of mesenchymal-like cells in the tumor or cancer cells of the patients having the tumor or cancer correspond to clinical non-response to the immunotherapy/immunotherapeutic compound, then the inhibitor of TCF4/ITF2 is administered to the patient having cancer. In all these cases, administration of the inhibitor of TCF4/ITF2 is advisable in order to increase, restore, or potentiate response to the immunotherapy/immunotherapeutic compound; or in order to increase sensitivity or decrease refraction to the immunotherapy/immunotherapeutic compound.

A further aspect of the invention relates to an inhibitor of transcription factor 4 (TCF4/ITF2) for use in (a method of/the manufacture of a medicament for) treating or inhibiting cancer, or for use in (a method of/the manufacture of a medicament for) inhibiting progression, relapse or metastasis of cancer, wherein the patient having cancer has received immunotherapy/immunotherapeutic compound, and has developed resistance to the immunotherapy/immunotherapeutic compound; and wherein the patient is subsequently receiving treatment comprising an inhibitor of BRAF in combination with an inhibitor of TCF4/ITF2, or is receiving treatment comprising a combination of an inhibitor of BRAF, an inhibitor of MEK, and an inhibitor of TCF4/ITF2.

In other words, the patient having cancer has received a first (or previous) line of treatment with an immunotherapy/immunotherapeutic compound; and may receive as a second (or subsequent) line of treatment a treatment comprising an inhibitor of BRAF in combination with an inhibitor of TCF4/ITF2, or is receiving treatment comprising a combination of an inhibitor of BRAF, an inhibitor of MEK, and an inhibitor of TCF4/ITF2.

In an alternative aspect, the invention relates to an immunotherapy for use in treating or inhibiting cancer or for use in inhibiting progression, relapse or metastasis of cancer, in combination with (administration of) an inhibitor of transcription factor 4 (TCF4/ITF2); or wherein the treatment or inhibition further comprises therapy with or administration of an inhibitor of transcription factor 4 (TCF4/ITF2); or wherein the treatment or inhibition is combined with a therapy comprising an inhibitor of transcription factor 4 (TCF4/ITF2) or with administration of an inhibitor of transcription factor 4 (TCF4/ITF2) (to the subject or individual having the cancer or tumor).

Alternatively, the invention relates to use of an immunotherapeutic compound or agent in the manufacture of a medicament for treating or inhibiting cancer or for inhibiting progression, relapse or metastasis of cancer (in a subject or individual having the cancer) in combination with an inhibitor of transcription factor 4 (TCF4/ITF2) (for treating or inhibiting cancer or for inhibiting progression, relapse or metastasis of cancer); or in combination with administering an inhibitor of transcription factor 4 (TCF4/ITF2) to the subject or individual; or wherein the treatment or inhibition further comprises therapy with or administration of an inhibitor of transcription factor 4 (TCF4/ITF2); or wherein the treatment or inhibition is combined with a therapy comprising an inhibitor of transcription factor 4 (TCF4/ITF2) or with administration of an inhibitor of transcription factor 4 (TCF4/ITF2) (to the subject or individual having the cancer or tumor).

In another alternative aspect, the invention relates to an inhibitor of transcription factor 4 (TCF4/ITF2) and an immunotherapy for use in treating or inhibiting cancer or for use in inhibiting progression, relapse or metastasis of cancer. Alternatively, the invention relates to use of an inhibitor of transcription factor 4 (TCF4/ITF2) and (use of) an immunotherapy/immunotherapeutic compound or agent in the manufacture of a medicament for use in treating or inhibiting cancer or for inhibiting progression, relapse or metastasis of cancer (in a subject or individual having the cancer).

A further aspect of the invention relates to a method for treating or inhibiting cancer, or a method for inhibiting progression, relapse or metastasis of cancer, in a subject or individual (in particular a mammalian subject or mammal, such as a human subject or human), the methods comprising administering an inhibitor of transcription factor 4 (TCF4/ITF2) and administering an immunotherapy/immunotherapeutic compound or agent to the subject or individual. By administering the inhibitor of transcription factor 4 (TCF4/ITF2) and the immunotherapy/immunotherapeutic compound or agent, the cancer is treated or inhibited, or the progression, relapse or metastasis of the cancer is inhibited. In particular, an effective amount of the inhibitor of transcription factor 4 (TCF4/ITF2) and of the immunotherapy/immunotherapeutic compound or agent is administered to the subject; or an effective amount of a combination (in any way) of the inhibitor of transcription factor 4 (TCF4/ITF2) and of the immunotherapy/immunotherapeutic compound or agent is administered to the subject.

In one embodiment relating to the immunotherapy/immunotherapeutic compound for any use as described above, the patient having cancer has received a prior treatment different from the immunotherapy/immunotherapeutic compound and different from the inhibitor of transcription factor 4 (TCF4/ITF2). In other words, the patient having cancer has received a first (or previous) line of treatment different from immunotherapy/immunotherapeutic compound and different from a TCF4/ITF2 inhibitor; and may receive as a second (or subsequent) line of treatment the immunotherapy/immunotherapeutic compound in combination with the TCF4/ITF2 inhibitor. In one embodiment thereto, the prior treatment (first line of treatment, or previous line of treatment) is treatment comprising an inhibitor of BRAF, or treatment with or comprising a combination of an inhibitor of BRAF and an inhibitor of MEK.

In yet a further aspect, the invention relates to a BET inhibitor for use in (a method of/the manufacture of a medicament for) treating or inhibiting cancer, or for use in (a method of/the manufacture of a medicament for) inhibiting progression, relapse or metastasis of cancer, wherein the patient having cancer has received immune checkpoint inhibitor therapy or a therapy comprising an immune checkpoint inhibitor, and has developed resistance to the therapy; and wherein the patient is subsequently receiving treatment comprising an inhibitor of BRAF in combination with a BET inhibitor, or is receiving treatment comprising an inhibitor of BRAF, an inhibitor of MEK, and a BET inhibitor.

In other words, the patient having cancer has received a first (or previous) line of treatment with an immunotherapy/immunotherapeutic compound; and may receive as a second (or subsequent) line of treatment comprising an inhibitor of BRAF in combination with a BET inhibitor, or is receiving treatment comprising an inhibitor of BRAF, an inhibitor of MEK, and a BET inhibitor.

In any of the above, the inhibitor of transcription factor 4 (TCF4/ITF2) may in particular be a specific inhibitor of transcription factor 4 (TCF4/ITF2) or a selective inhibitor of transcription factor 4 (TCF4/ITF2). Specificity and selectivity of inhibition are explained in more detail hereinafter. In particular, a specific or selective inhibitor of TCF4/ITF2 may be an inhibitory oligonucleotide specifically targeting TCF4/ITF2. Such inhibitory oligonucleotide specifically targeting TCF4/ITF2 may be selected from (the group consisting of) an antisense oligomer, a siRNA, a shRNA, a gapmer, and the likes. Furthermore, a specific or selective inhibitor of TCF4/ITF2 may be selected from (the group consisting of) DNA nucleases specifically knocking out or disrupting TCF4/ITF2, and RNases specifically targeting TCF4/ITF2. Such DNA nuclease specifically knocking out or disrupting TCF4/ITF2 may be selected from (the group consisting of) a ZFN, a TALEN, a CRISPR-Cas, and a meganuclease targeting TCF4/ITF2. Such RNase specifically targeting TCF4/ITF2 may be selected from (the group consisting of) a ribozyme and a CRISPR-C2c2.

In any of the above, the immunotherapy in one embodiment is a treatment or therapy with an immune checkpoint inhibitor or a treatment or therapy comprising an immune checkpoint inhibitor (the immunotherapeutic compound or agent in this case thus is an immune checkpoint inhibitor). In a further embodiment, the immunotherapy is a treatment or therapy with two immune checkpoint inhibitors or a treatment or therapy comprising two immune checkpoint inhibitors (the immunotherapeutic compound or agent in this case thus is a combination of two immune checkpoint inhibitors). In a particular embodiment, said two immune checkpoint inhibitors are then chosen such that each of the two inhibitors is inhibiting a different immune checkpoint protein or a different immune checkpoint protein-ligand interaction. The inhibitors of the two different immune checkpoint protein-ligand interactions are for instance a PD1 inhibitor and a CTLA4 inhibitor. The two different immune checkpoint-ligand interactions are for instance two selected from (the group consisting of) PD1 with ligand PDL1, PD1 with ligand PDL2, CTLA4 with ligand B7-1, CTLA4 with ligand B7-2.

In any of the above aspects and embodiments, the combination is in particular a combination in any way or in any appropriate way (explained in more detail hereinafter).

In any of the above aspects and embodiments, the tumor or cancer is, in a further embodiment, poorly responding to, resistant to, or refractory to immunotherapy or to therapy comprising an immunotherapeutic compound or agent. In a further embodiment, the tumor or cancer in particular is a tumor or cancer with a β-catenin defect. Such β-catenin defects include β-catenin mutations (in particular gain-of-function mutations) or any defect resulting in elevated β-catenin levels (Kolligs et al. 2002, Cancer Cell 1:145-155). In a particular embodiment, the tumor or cancer may be a breast, lung, colorectal, ovarian, or pancreatic tumor or cancer, may be a glioblastoma, or may be melanoma. In a further particular embodiment, the tumor or cancer is a metastatic tumor or cancer, such as metastatic melanoma.

The term “antagonist” or “inhibitor” of a target as used interchangeable herein refers to inhibitors of function or to inhibitors of expression of a target of interest. Interchangeable alternatives for “antagonist” include inhibitor, repressor, suppressor, inactivator, and blocker. An “antagonist” thus refers to a molecule that decreases, blocks, inhibits, abrogates, or interferes with target expression, activation, function, or activity.

Downregulating of expression of a gene encoding a target is feasible through gene therapy (e.g., by administering siRNA, shRNA or antisense oligonucleotides to the target gene). Biopharmaceutical and gene therapeutic antagonists include such entities as antisense oligonucleotides, gapmers, siRNA, shRNA, zinc-finger nucleases, meganucleases, TAL effector nucleases, CRISPR-Cas effectors, etc. (general description of these compounds included hereinafter).

Inactivation of a process as envisaged in the current invention refers to different possible levels of inactivation, e.g., at least 10%, at least 20%, at least 30%, at least 40%, at least 50%, at least 60%, at least 70%, at least 80%, at least 90%, at least 95%, or even 100% or more if inactivation (compared to a normal situation or compared to the situation prior to starting the inactivation). The nature of the inactivating compound is not vital/essential to the invention as long as the process envisaged is inactivated such as to treat or inhibit cancer or tumor growth or such as to inhibit progression, relapse or metastasis of cancer or tumor growth as described herein.

### Inhibition of a Target of Interest

Downregulating expression of a gene encoding a target is feasible through agents include entities such as antisense oligonucleotides, gapmers, siRNA, shRNA, zinc-finger nucleases, meganucleases, Argonaute, TAL effector nucleases, CRISPR-Cas effectors, and nucleic acid aptamers. In particular, any of these agents is specifically, selectively, or exclusively acting on or antagonizing the target of interest; or any of these agents is designed for specifically, selectively, or exclusively acting on or antagonizing the target of interest.

One process of modulating/downregulating expression of a gene/target gene of interest relies on antisense oligonucleotides (ASOs), or variants thereof such as gapmers. An antisense oligonucleotide (ASO) is a short strand of nucleotides and/or nucleotide analogues that hybridizes with the complementary mRNA in a sequence-specific or -selective manner. Formation of the ASO-mRNA complex ultimately results in downregulation of target protein expression (Chan et al. 2006, Clin Exp Pharmacol Physiol 33:533-540; this reference also describes some of the software available for assisting in design of ASOs). Modifications to ASOs can be introduced at one or more levels: phosphate linkage modification (e.g. introduction of one or more of phosphodiester, phosphoramidate or phosphorothioate bonds), sugar modification (e.g. introduction of one or more of LNA (locked nucleic acids), 2′-O-methyl, 2′-O-methoxy-ethyl, 2′-fluoro, S-constrained ethyl or tricyclo-DNA and/or non-ribose modifications (e.g. introduction of one or more of phosphorodiamidate morpholinos or peptide nucleic acids). The introduction of 2′-modifications has been shown to enhance safety and pharmacologic properties of antisense oligonucleotides. Antisense strategies relying on degradation of mRNA by RNase H requires the presence of nucleotides with a free 2′-oxygen, i.e. not all nucleotides in the antisense molecule should be 2′-modified. The gapmer strategy has been developed to this end. A gapmer antisense oligonucleotide consists of a central DNA region (usually a minimum of 7 or 8 nucleotides) with (usually 2 or 3) 2′-modified nucleosides flanking both ends of the central DNA region. This is sufficient for the protection against exonucleases while allowing RNAseH to act on the (2′-modification free) gap region. Antidote strategies are available as demonstrated by administration of an oligonucleotide fully complementary to the antisense oligonucleotide (Crosby et al. 2015, Nucleic Acid Ther 25:297-305). Another process to modulate expression of a gene/target gene of interest is based on the natural process of RNA interference. It relies on double-stranded RNA (dsRNA) that is cut by an enzyme called Dicer, resulting in double stranded small interfering RNA (siRNA) molecules which are 20-25 nucleotides long. siRNA then binds to the cellular RNA-Induced Silencing Complex (RISC) separating the two strands into the passenger and guide strand. While the passenger strand is degraded, RISC is cleaving mRNA specifically or selectively at a site instructed by the guide strand. Destruction of the mRNA prevents production of the protein of interest and the gene is ‘silenced’. siRNAs are dsRNAs with 2 nt 3′ end overhangs whereas shRNAs are dsRNAs that contains a loop structure that is processed to siRNA. shRNAs are introduced into the nuclei of target cells using a vector (e.g. bacterial or viral) that optionally can stably integrate into the genome. Apart from checking for lack of cross-reactivity with non-target genes, manufacturers of RNAi products provide guidelines for designing siRNA/shRNA. siRNA sequences between 19-29 nt are generally the most effective. Sequences longer than 30 nt can result in nonspecific silencing. Ideal sites to target include AA dinucleotides and the 19 nt 3′ of them in the target mRNA sequence. Typically, siRNAs with 3′ dUdU or dTdT dinucleotide overhangs are more effective. Other dinucleotide overhangs could maintain activity but GG overhangs should be avoided. Also to be avoided are siRNA designs with a 4-6 poly (T) tract (acting as a termination signal for RNA pol III), and the G/C content is advised to be between 35-55%. shRNAs should comprise sense and antisense sequences (advised to each be 19-21 nt in length) separated by loop structure, and a 3′ AAAA overhang. Effective loop structures are suggested to be 3-9 nt in length. It is suggested to follow the sense-loop-antisense order in designing the shRNA cassette and to avoid 5′ overhangs in the shRNA construct. shRNAs are usually transcribed from vectors, e.g. driven by the Pol III U6 promoter or H1 promoter. Vectors allow for inducible shRNA expression, e.g. relying on the Tet-on and Tet-off inducible systems commercially available, or on a modified U6 promoter that is induced by the insect hormone ecdysone. A Cre-Lox recombination system has been used to achieve controlled expression in mice. Synthetic shRNAs can be chemically modified to affect their activity and stability. Plasmid DNA or dsRNA can be delivered to a cell by means of transfection (lipid transfection, cationic polymer-based nanoparticles, lipid or cell-penetrating peptide conjugation) or electroporation. Vectors include viral vectors such as lentiviral, retroviral, adenoviral and adeno-associated viral vectors.

Ribozymes (ribonucleic acid enzymes) are another type of molecules that can be used to modulate expression of a gene/target gene of interest. They are RNA molecules capable of catalyzing specific biochemical reactions, in the current context capable of targeted cleavage of nucleotide sequences, in particular targeted cleavage of a RNA/RNA target of interest. Examples of ribozymes include the hammerhead ribozyme, the Varkud Satellite ribozyme, Leadzyme and the hairpin ribozyme.

Besides the use of the inhibitory RNA technology, modulation of expression of a gene of interest can be achieved at DNA level such as by gene therapy to knock-out, knock-down or disrupt the target gene/gene of interest. As used herein, a “gene knock-out” can be a gene knockdown or the gene can be knocked out, knocked down, disrupted or modified by a mutation such as, a point mutation, an insertion, a deletion, a frameshift, or a missense mutation by techniques such as described hereafter, including, but not limited to, retroviral gene transfer. One way in which genes can be knocked out, knocked down, disrupted or modified is by the use of zinc finger nucleases. Zinc-finger nucleases (ZFNs) are artificial restriction enzymes generated by fusing a zinc finger DNA-binding domain to a DNA-cleavage domain. Zinc finger domains can be engineered to target a desired DNA sequence/DNA sequence of interest, which enable zinc-finger nucleases to target unique sequence within a complex genome. By taking advantage of the endogenous DNA repair machinery, these reagents can be used to precisely alter the genomes of higher organisms.

Other technologies for genome customization that can be used to specifically or selectively knock out, knock down or disrupt a gene/gene of interest are meganucleases and TAL effector nucleases (TALENs, Cellectis bioresearch). A TALEN® is composed of a TALE DNA binding domain for sequence-specific or sequence-selective recognition fused to the catalytic domain of an endonuclease that introduces double strand breaks (DSB). The DNA binding domain of a TALEN® is capable of targeting with high precision a large recognition site (for instance 17 bp). Meganucleases are sequence-specific or sequence-selective endonucleases, naturally occurring “DNA scissors”, originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes) or DNA sequences of interest. Another recent genome editing technology is the CRISPR/Cas system, which can be used to achieve RNA-guided genome engineering (including knock-out, knock-down or disruption of a gene of interest).

CRISPR interference is a genetic technique which allows for sequence-specific or sequence-selective control of expression of a gene of interest in prokaryotic and eukaryotic cells. It is based on the bacterial immune system-derived CRISPR (clustered regularly interspaced palindromic repeats) pathway. Recently, it was demonstrated that the CRISPR-Cas editing system can also be used to target RNA. It has been shown that the Class 2 type VI-A CRISPR-Cas effector C2c2 (Cas13a; CRISPR-Cas13a or CRISPR-C2c2) can be programmed to cleave single stranded RNA targets carrying complementary protospacers (Abudayyeh et al. 2016 Science353/science.aaf5573). C2c2 is a single-effector endoRNase mediating ssRNA cleavage once it has been guided by a single crRNA guide toward a target RNA/RNA of interest.

Methods for administering nucleic acid-based therapeutic modalities/agents include methods applying non-viral (DNA or RNA) or viral nucleic acids (DNA or RNA viral vectors). Methods for non-viral gene therapy include the injection of naked DNA (circular or linear), electroporation, the gene gun, sonoporation, magnetofection, the use of oligonucleotides, lipoplexes (e.g. complexes of nucleic acid with DOTAP or DOPE or combinations thereof, complexes with other cationic lipids), dendrimers, viral-like particles, inorganic nanoparticles, hydrodynamic delivery, photochemical internalization (Berg et al. 2010, Methods Mol Biol 635:133-145) or combinations thereof.

Many different vectors have been used in human nucleic acid therapy trials and a listing can be found on http://www.abedia.com/wiley/vectors.php. Currently the major groups are adenovirus or adeno-associated virus vectors (in about 21% and 7% of the clinical trials), retrovirus vectors (about 19% of clinical trials), naked or plasmid DNA (about 17% of clinical trials), and lentivirus vectors (about 6% of clinical trials). Combinations are also possible, e.g. naked or plasmid DNA combined with adenovirus, or RNA combined with naked or plasmid DNA to list just a few. Other viruses (e.g. alphaviruses, vaccinia viruses such as vaccinia virus Ankara) are used in nucleic acid therapy and are not excluded in the context of the current invention.

Administration may be aided by specific formulation of the nucleic acid e.g. in liposomes (lipoplexes) or polymersomes (synthetic variants of liposomes), as polyplexes (nucleic acid complexed with polymers), carried on dendrimers, in inorganic (nano) particles (e.g. containing iron oxide in case of magnetofection), or combined with a cell penetrating peptide (CPP) to increase cellular uptake. Organ- or cellular-targeting strategies may also be applied to the nucleic acid (nucleic acid combined with organ- or cell-targeting moiety); these include passive targeting (mostly achieved by adapted formulation) or active targeting (e.g. by coupling a nucleic acid-comprising nanoparticle with any compound (e.g. an aptamer or antibody or antigen binding molecule) binding to a target organ- or cell-specific antigen) (e.g. Steichen et al. 2013, Eur J Pharm Sci 48:416-427).

CPPs enable translocation of their payload of interest across the plasma membrane. CPPs are alternatively termed Protein Transduction Domains (TPDs), usually comprise 30 or less (e.g. 5 to 30, or 5 to 20) amino acids, and usually are rich in basic residues, and are derived from naturally occurring CPPs (usually longer than 20 amino acids), or are the result of modelling or design. A non-limiting selection of CPPs includes the TAT peptide (derived from HIV-1 Tat protein), penetratin (derived from Drosophila Antennapedia-Antp), pVEC (derived from murine vascular endothelial cadherin), signal-sequence based peptides or membrane translocating sequences, model amphipathic peptide (MAP), transportan, MPG, polyarginines; more information on these peptides can be found in Torchilin 2008 (Adv Drug Deliv Rev 60:548-558) and references cited therein. CPPs can be coupled to carriers such as nanoparticles, liposomes, micelles, or generally any hydrophobic particle. Coupling can be by absorption or chemical bonding, such as via a spacer between the CPP and the carrier. To increase target specificity or target selectivity, an antibody binding to a target-specific antigen can further be coupled to the carrier (Torchilin 2008, Adv Drug Deliv Rev 60:548-558). CPPs have already been used to deliver payloads as diverse as plasmid DNA, oligonucleotides, siRNA, peptide nucleic acids (PNA), proteins and peptides, small molecules and nanoparticles inside the cell (Stalmans et al. 2013, PloS One 8: e71752).

Any other modification of the DNA or RNA to enhance efficacy of nucleic acid therapy is likewise envisaged to be useful in the context of the applications of the genetic inhibitor as outlined herein. The enhanced efficacy can reside in enhanced expression, enhanced delivery properties, enhanced stability and the like. The applications of the genetic inhibitor as outlined herein may thus rely on using a modified nucleic acid as described above. Further modifications of the nucleic acid may include those suppressing inflammatory responses (hypoinflammatory nucleic acids).

A specific or selective inhibitor of a target of interest may exert the desired level of inhibition of the target of interest with an IC50 of 1000 nM or less, with an IC50 of 500 nM or less, with an IC50 of 100 nM or less, with an IC50 of 50 nM or less, with an IC50 of 10 nM or less, with an IC50 of 1 nM or less, with an IC50 between 1 pM and 1 nM, or with an IC50 between 0.1 pM and 10 nM.

Cross-inhibition of more than one target is possible; for clinical development it can e.g. be desired to be able to test an inhibitor in a suitable in vitro model or in vivo animal model before starting clinical testing with the same inhibitor in a human population, which may require the inhibitor to cross-inhibit the animal (or other non-human) target and the orthologous human target.

Specificity or selectivity of inhibition refers to the situation in which an inhibitor is, at a certain concentration (sufficient to inhibit the target of interest) inhibiting the target protein with higher efficacy (e.g. with an at least 2-fold, 5-fold, or 10-fold lower IC50, e.g. at least 20-, 50- or 100-fold or more lower IC50) than the efficacy with which it is possibly (if at all) inhibiting other targets (targets not of interest). Such specificity or selectivity of inhibition is in particular determined within the setting of the target subject (e.g. human patient, or animal model) and thus can encompass/does not exclude inhibition of (at least one) orthologous target. Exclusivity of inhibition refers to the situation in which an inhibitor is inhibiting only the target of interest.

### Treatment/Therapeutically Effective Amount

The terms therapeutic modality, therapeutic agent, agent, and drug are used interchangeably herein, and likewise relate to immunotherapeutic compounds or agents. All refer to a therapeutically active compound, to a combination of therapeutically active compounds, or to a therapeutically active composition (comprising one or more therapeutically active compounds).

“Treatment”/“treating” refers to any rate of reduction, delaying or retardation of the progress of the disease or disorder, or a single symptom thereof, compared to the progress or expected progress of the disease or disorder, or single symptom thereof, when left untreated. This implies that a therapeutic modality on its own may not result in a complete or partial response (or may even not result in any response), but may, in particular when combined with other therapeutic modalities (such as, but not limited thereto: surgery, radiation, etc.), contribute to a complete or partial response (e.g. by rendering the disease or disorder more sensitive to therapy). More desirable, the treatment results in no/zero progress of the disease or disorder, or single symptom thereof (i.e. “inhibition” or “inhibition of progression”), or even in any rate of regression of the already developed disease or disorder, or single symptom thereof. “Suppression/suppressing” can in this context be used as alternative for “treatment/treating”. Treatment/treating also refers to achieving a significant amelioration of one or more clinical symptoms associated with a disease or disorder, or of any single symptom thereof. Depending on the situation, the significant amelioration may be scored quantitatively or qualitatively. Qualitative criteria may e.g. by patient well-being. In the case of quantitative evaluation, the significant amelioration is typically a 10% or more, a 20% or more, a 25% or more, a 30% or more, a 40% or more, a 50% or more, a 60% or more, a 70% or more, a 75% or more, a 80% or more, a 95% or more, or a 100% improvement over the situation prior to treatment. The time-frame over which the improvement is evaluated will depend on the type of criteria/disease observed and can be determined by the person skilled in the art.

A “therapeutically effective amount” refers to an amount of a therapeutic agent to treat, inhibit or prevent a disease or disorder in a subject (such as a mammal). In the case of cancers, the therapeutically effective amount of the therapeutic agent may reduce the number of cancer cells; reduce the primary tumor size; inhibit (i.e., slow down to some extent and preferably stop) cancer cell infiltration into peripheral organs; inhibit (i.e., slow down to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, e.g., be measured by assessing the duration of survival (e.g. overall survival), time to disease progression (TTP), response rates (e.g., complete response and partial response, stable disease), length of progression-free survival (PFS), duration of response, and/or quality of life.

The term “effective amount” or “therapeutically effective amount” may depend on the dosing regimen of the agent/therapeutic agent or composition comprising the agent/therapeutic agent (e.g. medicament or pharmaceutical composition). The effective amount will generally depend on and/or will need adjustment to the mode of contacting or administration. The effective amount of the agent or composition comprising the agent is the amount required to obtain the desired clinical outcome or therapeutic effect without causing significant or unnecessary toxic effects (often expressed as maximum tolerable dose, MTD). To obtain or maintain the effective amount, the agent or composition comprising the agent may be administered as a single dose or in multiple doses. The effective amount may further vary depending on the severity of the condition that needs to be treated; this may depend on the overall health and physical condition of the subject or patient and usually the treating doctor's or physician's assessment will be required to establish what is the effective amount. The effective amount may further be obtained by a combination of different types of contacting or administration.

The aspects and embodiments described above in general may comprise the administration of one or more therapeutic compounds to a subject (such as a mammal) in need thereof, i.e., harboring a tumor, cancer or neoplasm in need of treatment. In general a (therapeutically) effective amount of (a) therapeutic compound(s) is administered to the mammal in need thereof in order to obtain the described clinical response(s).

“Administering” means any mode of contacting that results in interaction between an agent (e.g. a therapeutic compound or immunotherapeutic compound or agent) or composition comprising the agent (such as a medicament or pharmaceutical composition) and an object (e.g. cell, tissue, organ, body lumen) with which said agent or composition is contacted. The interaction between the agent or composition and the object can occur starting immediately or nearly immediately with the administration of the agent or composition, can occur over an extended time period (starting immediately or nearly immediately with the administration of the agent or composition), or can be delayed relative to the time of administration of the agent or composition. More specifically the “contacting” results in delivering an effective amount of the agent or composition comprising the agent to the object.

### Combinations

The invention further relates to a combination of an inhibitor of transcription factor 4 (TCF4/ITF2) and an immunotherapeutic compound or agent. Alternatively, the invention relates to a combination of a composition, such as a pharmaceutically acceptable composition, comprising an inhibitor of transcription factor 4 (TCF4/ITF2); and of a composition, such as a pharmaceutically acceptable composition, comprising an immunotherapeutic compound or agent. In one embodiment thereto, the invention relates to a combination of an inhibitor of transcription factor 4 (TCF4/ITF2) and an immunotherapeutic compound or agent which is an immune checkpoint inhibitor.

In a further embodiment thereto, the invention relates to a combination of an inhibitor of transcription factor 4 (TCF4/ITF2) and an immunotherapeutic compound or agent which is a combination of two immune checkpoint inhibitors. In the latter case a further embodiment relates to a combination of a composition, such as a pharmaceutically acceptable composition, comprising an inhibitor of transcription factor 4 (TCF4/ITF2); of a composition, such as a pharmaceutically acceptable composition, comprising a first immune checkpoint inhibitor; and of a composition, such as a pharmaceutically acceptable composition, comprising a second immune checkpoint inhibitor.

The invention further relates to any composition comprising a combination of an inhibitor of transcription factor 4 (TCF4/ITF2) and an immunotherapeutic compound or agent as described hereinabove for use as a medicine or medicament. Alternatively, the invention relates to a medicine or medicament comprising a combination of an inhibitor of transcription factor 4 (TCF4/ITF2) and an immunotherapeutic compound or agent as described hereinabove. In one embodiment thereto, these combinations, compositions, medicines or medicaments are for use in treating or inhibiting cancer, or for use in inhibiting progression, relapse or metastasis of cancer. In one embodiment, the cancer is poorly responding to, resistant to, or refractory to immunotherapy or to therapy comprising an immunotherapeutic compound or agent.

Any of these combinations, compositions, medicines or medicaments may further be combined with another anti-cancer treatment or therapy such as surgery, radiation, chemotherapy etc.

“Combination”, “combination in any way” or “combination in any appropriate way” as referred to herein is meant to refer to any sequence of administration of two (or more) therapeutic modalities, i.e. the administration of the two (or more) therapeutic modalities can occur concurrently in time or separated from each other by any amount of time; and/or “combination”, “combination in any way” or “combination in any appropriate way” as referred to herein can refer to the combined or separate formulation of the two (or more) therapeutic modalities, i.e. the two (or more) therapeutic modalities can be individually provided in separate vials or (other suitable) containers, or can be provided combined in the same vial or (other suitable) container. When combined in the same vial or (other suitable) container, the two (or more) therapeutic modalities can each be provided in the same vial/container chamber of a single-chamber vial/container or in the same vial/container chamber of a multi-chamber vial/container; or can each be provided in a separate vial/container chamber of a multi-chamber vial/container.

### Therapeutic Kits

The invention further relates to (therapeutic) kits comprising a container or vial (any suitable container or vial, such as a pharmaceutically acceptable container or vial) comprising an inhibitor of transcription factor 4 (TCF4/ITF2) or comprising a composition comprising an inhibitor of transcription factor 4 (TCF4/ITF2); and optionally comprising a container or vial (any suitable container or vial, such as a pharmaceutically acceptable container or vial) comprising an immunotherapeutic compound or agent, such as an immune checkpoint inhibitor. One embodiment relates to kits comprising a container or vial (any suitable container or vial, such as a pharmaceutically acceptable container or vial) comprising an inhibitor of transcription factor 4 (TCF4/ITF2) or comprising a composition comprising an inhibitor of transcription factor 4 (TCF4/ITF2); and optionally: comprising a first immune checkpoint inhibitor or a composition comprising a first immune checkpoint inhibitor and comprising a container or vial (any suitable container or vial, such as a pharmaceutically acceptable container or vial) comprising a second immune checkpoint inhibitor or a composition comprising a second immune checkpoint inhibitor.

Alternatively, such kits are comprising a container or vial (any suitable container or vial, such as a pharmaceutically acceptable container or vial) comprising a combination of an inhibitor of transcription factor 4 (TCF4/ITF2) and an immunotherapeutic compound or agent (such as one or two immune checkpoint inhibitors) (see discussion on “combination in any way” on how such combination in a single container, e.g., vial can be defined). Other optional components of such kit include one or more diagnostic agents capable of predicting, prognosing, or determining the success of a therapy comprising one of the therapies according to the invention; use instructions; one or more containers with sterile pharmaceutically acceptable carriers, excipients or diluents [such as for producing or formulating a (pharmaceutically acceptable) composition of the invention]; one or more syringes; one or more needles; etc. In particular, such kits may be pharmaceutical kits.

Immune checkpoints antagonists or inhibitors as referred to herein include the cell surface protein cytotoxic T lymphocyte antigen-4 (CTLA-4), programmed cell death protein-1 (PD-1) and their respective ligands. CTLA-4 binds to its co-receptor B7-1 (CD80) or B7-2 (CD86); PD-1 binds to its ligands PD-L1 (B7-H10) and PD-L2 (B7-DC). Other immune checkpoint inhibitors include the adenosine A2A receptor (A2AR), B7-H3 (or CD276), B7-H4 (or VTCN1), BTLA (or CD272), IDO (indoleamine 2,3-10 dioxygenase), KIR (killer-cell immunoglobulin-like receptor), LAG3 (lymphocyte activation gene-3), NOX2 (nicotinamide adenine dinucleotide phosphate (NADPH) oxidase isoform 2), TIM3 (T-cell immunoglobulin domain and mucin domain 3), VISTA (V-domain lg suppressor of T cell activation), SIGLEC7 (sialic acid-binding immunoglobulin-type lectin 7, or CD328) and SIGLEC9 (sialic acid-binding immunoglobulin-type lectin 9, or CD329).

In any of the above methods, embodiments, and kits, referring to two immune checkpoint inhibitors, in one embodiment these are each inhibiting a different immune checkpoint or a different immune checkpoint-ligand interaction. For instance, when an inhibitor of PD1 is selected as a first immune checkpoint inhibitor, the second immune checkpoint inhibitor could be an inhibitor of PDL1 or an inhibitor of PDL2. Such first and second immune checkpoint inhibitor are each inhibiting a different immune checkpoint protein. In a further non-limiting example, an inhibitor of PD1 is selected as a first immune checkpoint inhibitor, and as second immune checkpoint inhibitor an inhibitor different from an inhibitor of PDL1 and different from an inhibitor of PDL2 is selected, e.g. an inhibitor of CTLA-4 is selected. In this latter example, the first and second immune checkpoint inhibitor are not only each inhibiting a different immune checkpoint, but also each inhibiting a different immune checkpoint-ligand interaction.

### Immunotherapy/Immunotherapeutic Compound or Agent

Immunotherapy in general is defined as a treatment comprising administration of an immunotherapeutic compound or agent that supports (including activation or reactivation) the body's own immune system to help fight a disease, more specifically cancer in the context of the current invention. Immunotherapeutic treatment as used herein refers to the reactivation and/or stimulation and/or reconstitution of the immune response of a mammal towards a condition such as a tumor, cancer or neoplasm evading and/or escaping and/or suppressing normal immune surveillance. The reactivation and/or stimulation and/or reconstitution of the immune response of a mammal in turn in part results in an increase in elimination of tumorous, cancerous or neoplastic cells by the mammal's immune system (anticancer, antitumor or anti-neoplasm immune response; adaptive immune response to the tumor, cancer or neoplasm).

Immunotherapeutic agents include antibodies, in particular monoclonal antibodies, employed as (targeted) anti-cancer agents include alemtuzumab (chronic lymphocytic leukemia), bevacizumab (colorectal cancer), cetuximab (colorectal cancer, head and neck cancer), denosumab (solid tumor's bony metastases), gemtuzumab (acute myelogenous leukemia), ipilumab (melanoma), ofatumumab (chronic lymphocytic leukemia), panitumumab (colorectal cancer), rituximab (Non-Hodgkin lymphoma), tositumomab (Non-Hodgkin lymphoma) and trastuzumab (breast cancer). Other antibodies include for instance abagovomab (ovarian cancer), adecatumumab (prostate and breast cancer), afutuzumab (lymphoma), amatuximab, apolizumab (hematological cancers), blinatumomab, cixutumumab (solid tumors), dacetuzumab (hematologic cancers), elotuzumab (multiple myeloma), farletuzumab (ovarian cancer), intetumumab (solid tumors), muatuzumab (colorectal, lung and stomach cancer), onartuzumab, parsatuzumab, pritumumab (brain cancer), tremelimumab, ublituximab, veltuzumab (non-Hodgkin's lymphoma), votumumab (colorectal tumors), zatuximab and anti-placental growth factor antibodies such as described in WO 2006/099698.

Immunotherapeutic agents of particular interest further include immune checkpoint inhibitors (such as anti-PD-1, anti-PD-L1 or anti-CTLA-4 antibodies; detailed hereinafter), bispecific antibodies bridging a cancer cell and an immune cell, dendritic cell vaccines, CAR-T cells, oncolytic viruses, RNA vaccines, and so on. Immunotherapy is a promising new area of cancer therapeutics and several immunotherapies are being evaluated preclinically as well as in clinical trials and have demonstrated promising activity (Callahan et al. 2013, J Leukoc Biol 94:41-53; Page et al. 2014, Annu Rev Med 65:185-202). However, not all the patients are sensitive to immune checkpoint blockade and sometimes PD-1 or PD-L1 blocking antibodies accelerate tumor progression. An overview of clinical developments in the field of immune checkpoint therapy is given by Fan et al. 2019 (Oncology Reports 41:3-14). Combinatorial cancer treatments that include chemotherapies can achieve higher rates of disease control by impinging on distinct elements of tumor biology to obtain synergistic antitumor effects. It is now accepted that certain chemotherapies can increase tumor immunity by inducing immunogenic cell death and by promoting escape in cancer immunoediting, such therapies are therefore called immunogenic therapies as they provoke an immunogenic response. Drug moieties known to induce immunogenic cell death include bleomycin, bortezomib, cyclophosphamide, doxorubicin, epirubicin, idarubicin, mafosfamide, mitoxantrone, oxaliplatin, and patupilone (Bezu et al. 2015, Front Immunol 6:187). Other forms of immunotherapy include chimeric antigen receptor (CAR) T-cell therapy in which allogenic T-cells are adapted to recognize a tumoral neo-antigen and oncolytic viruses preferentially infecting and killing cancer cells. Treatment with RNA, e.g. encoding MLKL, is a further means of provoking an immunogenic response (Van Hoecke et al. 2018, Nat Commun 9:3417), as well as vaccination with neo-epitopes (Brennick et al. 2017, Immunotherapy 9:361-371).

### Inhibitors of BRAF, MEK, and BET

BRAF (aliases: B-Raf Proto-Oncogene; Serine/Threonine Kinase; V-Raf Murine Sarcoma Viral Oncogene Homolog B1; V-Raf Murine Sarcoma Viral Oncogene Homolog B; Proto-Oncogene B-Raf; BRAF1; RAFB1; B-Raf Proto-Oncogene Serine/Threonine-Protein Kinase (P94); Murine Sarcoma Viral (V-Raf) Oncogene Homolog B1; Serine/Threonine-Protein Kinase B-Raf; B-Raf Serine/Threonine-Protein; 94 KDa B-Raf Protein; EC 2.7.11.1; B-RAF1, B-Raf; NS7; P94); NCBI reference mRNA sequences include: NM_001354609.1, NM_004333.5, NM_001354609, XM_017012558.1, XM_017012559.1 MEK1 (aliases: Mitogen-Activated Protein Kinase Kinase 1; ERK Activator Kinase 1; MAPK/ERK Kinase 1; EC 2.7.12.2; MAPKK 1; PRKMK1; MAP2K1; MEK1; MKK1; Mitogen-Activated, Kinase 1; Dual Specificity Mitogen-Activated Protein Kinase Kinase 1; MAP Kinase Kinase 1; MAPKK; CFC3); NCBI reference mRNA sequences include: NM_002755.3, XM_011521783.2, XM_017022411.1, XM_017022412.1, XM_017022413.1

MEK2 (aliases: Mitogen-Activated Protein Kinase Kinase 2, ERK Activator Kinase 2, MAP Kinase Kinase 2, MAPK/ERK Kinase 2, EC 2.7.12.2, PRKMK2, MAP2K2, MKK2, Dual Specificity Mitogen-Activated Protein Kinase Kinase 2, MAPKK2, CFC4); NCBI reference cDNA sequences include NM_030662.3, XM_006722799.2, XM_017026989.1, XM_017026990.1, XM_017026991.1.

The term “MAPK signaling pathway” refers to the mitogen-activated protein kinase signaling pathway (e.g., the RAS/RAF/MEK/ERK signaling pathway) which encompasses a family of conserved serine/threonine protein kinases (e.g., the mitogen-activated protein kinases (MAPKs)). Abnormal regulation of the MAPK pathway contributes to uncontrolled proliferation, invasion, metastases, angiogenesis, and diminished apoptosis. The RAS family of GTPases includes KRAS, HRAS, and NRAS. Exemplary MAPKs within the RAS/RAF/MEK/ERK signaling pathway include the RAF family of serine/threonine protein kinases (such as ARAF, BRAF, and CRAF (RAF1)) and the extracellular signal-regulated kinase 1 and 2 (i.e., ERK1 and ERK2). The importance of the MAPK pathway in melanoma is recognized in the art. It is estimated that about 40-60% of melanomas carry a mutation in this MAPK pathway which leads to constitutive activation of the MAPK pathway. The mutation is often in the BRAF gene, more particularly BRAF V600E and V600K (20% of MAPK mutations in melanoma), or V600R (7% of MAPK mutations in melanoma). Exclusive to BRAF mutations, mutations in the N-RAS gene occur (20% of MAPK mutations in melanoma; one N-RAS mutant in melanoma results in NRAS Q61K mutant kinase protein). Mutations in cKIT, GNAQ and GNA11 are not frequent in cutaneous melanoma but cKIT (mast/stem cell growth factor receptor (SCFR), proto-oncogene c-Kit, tyrosine-protein kinase Kit, CD117) mutations occur with a 20-30% incidence in mucosal melanomas, and mutations in GNAQ (guanine nucleotide-binding protein G (q) subunit alpha) and GNA11 (GNAQ paralogue) with a 85% incidence in uveal melanoma. (Manzano et al. 2016, Ann Transl Med 4:237). Furthermore, MEK1 (MAP2K1) mutants include MEK P124L identified in a melanoma patient and may confer cross-resistance to B-RAF inhibition (Emery et al. 2009, Proc Natl Acad Sci USA 106:20411-20416). Other BRAF mutations include BRAF G593S, BRAF L597R and BRAF K601E, whereas other MEK1 mutations include F53L, P124S, E203K and N382H, and MEK2 mutations include S154F (Nikolaev et al. 2012, Nature Genet 44:133-139). A comprehensive review of BRAF mutations in melanoma and other cancers is provided by Dankner et al. 2018 (Oncogene 37:3183-3199).

The term “inhibitor of MAPK pathway”, “MAPK signaling inhibitor”, “MAPK pathway inhibitor” or “MAPK pathway signaling inhibitor” (wherein “inhibitor” may be exchanged for “antagonist”) refers to a molecule that decreases, blocks, inhibits, abrogates, or interferes with signal transduction through the MAPK pathway (e.g., the RAS/RAF/MEK/ERK pathway). In some embodiments, a MAPK signaling inhibitor may inhibit the activity of one or more proteins involved in the activation of MAPK signaling. In some embodiments, a MAPK signaling inhibitor may activate the activity of one or more proteins involved in the inhibition of MAPK signaling. MAPK signaling inhibitors include, but are not limited to, MEK inhibitors (e.g., MEK1 inhibitors, MEK2 inhibitors, and inhibitors of both MEK1 and MEK2), RAF inhibitors (e.g., ARAF inhibitors, BRAF inhibitors, CRAF inhibitors, and pan-RAF inhibitors (i.e., RAF inhibitors that are inhibiting more than one member of the RAF family (i.e., two or all three of ARAF, BRAF, and CRAF)), and ERK inhibitors (e.g., ERK1 inhibitors and ERK2 inhibitors).

The term “inhibitor of BRAF or CRAF”, “inhibitor of BRAF or CRAF kinase”, or “BRAF or CRAF inhibitor” (wherein “inhibitor” may be exchanged for “antagonist”) refers to molecule that decreases, blocks, inhibits, abrogates, or interferes with BRAF or CRAF activation or function.

Sorafenib (a tyrosine kinase inhibitor, TKI) blocks wild-type BRAF, whereas vemurafenib (Zelboraf®) and dabrafenib (Tafinlar®) block mutant BRAF (B-RAF kinase) protein. Phase 3 trial data indicated a rapid response of BRAF-mutant melanoma to vemurafenib but of a short duration. BRAF-inhibitors are also referred to as RAF-inhibitors. Other examples of BRAF inhibitors include, without limitation, sorafenib, vemurafenib, dabrafenib, regorafenib, LY-3009120, HM95573, LXH-254, MLN2480, BeiGene-283, RXDX-105, BAL3833, encorafenib (LGX818), GDC-0879, XL281, ARQ736, PLX3603, RAF265, selumetinib, trametinib, cobimetinib, pimasertib, refametinib, binimetinib, CI-1040 (PD184352), GDC-0623, PD-0325901, and BI-847325, or a pharmaceutically acceptable salt of any thereof. BRAF inhibitors may inhibit only BRAF or may inhibit BRAF and one or more additional targets. BRAF inhibitors are described in e.g. WO 2005/062795, WO 2007/002325, WO 2007/002433, WO 2008/079903, and WO 2008/079906.

Examples of CRAF include, without limitation, sorafenib, semapimod (Messoussi et al. 2014, Chem Biol 21:1433-1443), or a pharmaceutically acceptable salt thereof. CRAF inhibitors may inhibit only CRAF or may inhibit CRAF and one or more additional targets.

The term “pan-RAF inhibitor” (wherein “inhibitor” may be exchanged for “antagonist”) refers to a molecule that decreases, blocks, inhibits, abrogates, or interferes with the activation or function of two or more RAF family members (e.g., two or more of ARAF, BRAF, and CRAF). In one embodiment, the pan-RAF inhibitor inhibits all three RAF family members (i.e., ARAF, BRAF, and CRAF) to some extent. Examples of pan-RAF inhibitors include, without limitation, LY-3009120, HM95573, LXH-254, MLN2480, BeiGene-283, RXDX-105, BAL3833, regorafenib, and sorafenib, or a pharmaceutically acceptable salt thereof. Pan-RAF inhibitors are described in e.g. WO2013/100632, WO2014/151616, and WO2015/075483. Pan-RAF inhibitors may inhibit ARAF, BRAF, and/or CRAF and one or more additional targets.

MEK inhibitors seem less effective compared to BRAF inhibitors. A common approach becoming standard of care, is to combine BRAF- and MEK-inhibitors. Such combination often is effective over a longer time and seems to reduce the frequency of side effects such as the development of squamous cell skin cancers. The term “MEK inhibitor” (wherein “inhibitor” may be exchanged for “antagonist”) refers to molecule that decreases, blocks, inhibits, abrogates, or interferes with MEK (e.g., MEK1 and/or MEK2) activation or function. MEK inhibitors include selumetinib, trametinib (Mekinist®), cobimetinib (Cotellic®, hemifumarate salt of cobimetinib), pimasertib, refametinib, binimetinib, and CI-1040 (PD184352), or a pharmaceutically acceptable salt thereof. Other examples of MEK inhibitors include, without limitation, GDC-0623, PD-0325901, and BI-847325, or a pharmaceutically acceptable salt thereof. MEK inhibitors are described in e.g. WO 2007/044515, WO 2008/024725, WO 2008/024724, WO 2008/067481, WO 2008/157179, WO 2009/085983, WO 2009/085980, WO 2009/082687, WO 2010/003025, and WO 2010/003022. MEK inhibitors may inhibit only MEK or may inhibit MEK and one or more additional targets.

BET proteins are reviewed by Tanigeuchi 2016 (Int J Mol Sci 17:1849), and include human BRD2, BRD3, BRD4 and BRDT. A BET antagonist may inhibit one or more of the paralogous BET proteins. At least five BET-antagonistic pharmacologic compounds are in clinical trials: RVX-208, I-BET762 (GSK525762A), OTX 015, CPI0610 and TEN-010. Other small molecule BET antagonists include JQ1, I-BET151, I-BET, CPI203, RVX2135, dinaciclib, PFI-1, and RVX-208 (Fu et al. 2015, Oncotarget 6:5501-5516).

### TCF4, TCF7L2, PD1, PD-L1, CTLA4

### TCF4/ITF2

Entrez ID 6925. Aliases of TCF4 provided in GeneCards® include Transcription Factor 4; ITF2 or ITF-2 or immunoglobulin transcription factor 2; E2-2; and BHLHB19 or Basic Helix Loop Helix Protein 19. The genomic locations for the TCF4/ITF2 gene are chr18: 55,222,185-55,664,787 (GRCh38/hg38), minus strand; chr18: 52,889,416-53,303,188 (GRCh37/hg19 by Entrez Gene), minus strand; or chr18: 52,889,562-53,332,018 (GRCh37/hg19 by Ensembl), minus strand. GenBank reference TCF4/ITF2 mRNA sequences include accession nos. NM_001083962.2; NM_001243226.3; NM_001243227.2; NM_001243228.2; and NM_001243230.2. TCF4/ITF2 human shRNA lentiviral particles are offered for sale by e.g. Origene. Other TCF4/ITF2 siRNA and shRNA products are available through e.g. Santa Cruz Biotechnology.

### TCF7L2

Aliases of TCF7L2 provided in GeneCards® include Transcription Factor 7 like 2; T-cell factor 4 or TCF4 or T-cell specific transcription factor 4. The genomic locations for the TCF4/ITF2 gene are chr10: 112,950,247-113,167,678 (GRCh38/hg38), plus strand; chr 10:114,710,006-114,927,437 (GRCh37/hg19 by Entrez Gene), plus strand; or chr10: 114,710,009-114,927,437 (GRCh37/hg19 by Ensembl), plus strand. GenBank reference TCF7L2 mRNA sequences include accession nos. NM_001146274.2; NM_001146283.2; NM_001146284.2; NM_001146285.2; and NM_001146286.2.

### PD1

Aliases of PD1 provided in GeneCards® include PDCD1; Programmed Cell Death 1; Systemic Lupus Erythematosus Susceptibility 2; PD-1; CD279; HPD-1; SLEB2; and HPD-L. The genomic locations for the PDCD1 gene are chr2: 241,849,881-241,858,908 (in GRCh38/hg38) and chr2: 242,792,033-242,801,060 (in GRCh37/hg19). The GenBank reference PD1 mRNA sequence is known under accession no. NM_005018.3. Approved PD1-inhibiting antibodies include nivolumab, pembrolizumab, and cemiplimab; PD1-inhibiting antibodies under development include CT-011 (pidilizumab) and therapy with PD1-inhibiting antibodies is referred to herein as α-PD-1 therapy or α-PD1 therapy. PD1 siRNA and shRNA products are available through e.g. Origene.

### PD-L1

Aliases of PD-L1 provided in GeneCards® include CD274, Programmed Cell Death 1 Ligand 1, B7 Homolog 1, B7H1, PDL1, PDCD1 Ligand 1, PDCD1LG1, PDCD1L1, HPD-L1, B7-H1, B7-H, and Programmed Death Ligand 1. The genomic locations for the PDCD1 gene are chr9: 5,450,503-5,470,567 (in GRCh38/hg38) and chr9: 5,450,503-5,470,567 (in GRCh37/hg19). The GenBank reference PD1 mRNA sequence is known under accession no. NM 001267706.1, NM 001314029.2 and NM 014143.4. Approved PD-L1-inhibiting antibodies include atezolizumab, avelumab, and durvalumab. PD-L1 siRNA and shRNA products are available through e.g. Origene.

### CTLA4

Aliases of CTLA4 provided in GeneCards® include Cytotoxic T-Lymphocyte Associated Protein 4; CTLA-4; CD152; Insulin-Dependent Diabetes Mellitus 12; Cytotoxic T-Lymphocyte Protein 4; Celiac Disease 3; GSE; Ligand And Transmembrane Spliced Cytotoxic T Lymphocyte Associated Antigen 4; Cytotoxic T Lymphocyte Associated Antigen 4 Short Spliced Form; Cytotoxic T-Lymphocyte-Associated Serine Esterase-4; Cytotoxic T-Lymphocyte-Associated Antigen 4; CELIAC3; IDDM12; ALPS5; and GRD4. The genomic locations for the CTLA4 gene are chr2: 203,867,771-203,873,965 (in GRCh38/hg38) and chr2: 204,732,509-204,738,683 (in GRCh37/hg19). The GenBank reference CTLA4 mRNA sequences are known under accession nos. NM_001037631.3 and NM_005214.5. Approved CTLA4-inhibiting antibodies include ipilumab; CTLA4-inhibiting antibodies under development include tremelimumab; therapy with CTLA4-inhibiting antibodies is referred to herein as α-CTLA4 therapy. CTLA4 siRNA and shRNA products are available through e.g. Origene.

### Determining Response to Immune Checkpoint Blockade

In a further aspect, the invention relates to methods for determining or predicting (clinical) response of a cancer patient to immunotherapy or to therapy comprising an immunotherapeutic compound or agent, such methods comprising:
determining, assessing, measuring, or quantifying the level of TCF4/ITF2 expression in a sample obtained from a cancer lesion, wherein the sample is obtained after the patient having cancer received at least one administration of an immunotherapy or of the therapy comprising an immunotherapeutic compound or agent;
determining or predicting the cancer patient having cancer to (clinically) respond to the immunotherapy or to the therapy comprising an immunotherapeutic compound or agent when the TCF4/ITF2 expression level in the sample corresponds to TCF4/ITF2 expression levels in the same type of cancer of patients known to (clinically) respond to the immunotherapy or to the therapy comprising an immunotherapeutic compound or agent.

TCF4/ITF2 expression levels in the same type of cancer of patients known to (clinically) respond to the immunotherapy or to the therapy comprising an immunotherapeutic compound or agent herein form the reference TCF4/ITF2 expression levels, in particular the responder reference TCF4/ITF2 expression levels, to which a test sample's TCF4/ITF2 expression levels are compared.

TCF4/ITF2 expression levels in the same type of cancer of patients known not to (clinically) respond to the immunotherapy or to the therapy comprising an immunotherapeutic compound or agent in general will also be available, and form another set of reference TCF4/ITF2 expression levels, in particular the non-responder reference TCF4/ITF2 expression levels.

Non-responder reference TCF4/ITF2 expression levels and responder reference TCF4/ITF2 expression levels each can be expressed as minimum to maximum expression levels, as average expression levels, or as average+/−standard deviation expression levels; and each are typically derived from a plurality of treated patients for which the (clinical) response has been determined/is known.

Non-responder reference TCF4/ITF2 expression levels and responder reference TCF4/ITF2 expression levels are numerically distinguishable from each other and a test sample's TCF4/ITF2 expression level will “correspond” to either one, i.e., will either be close to either reference expression levels or will be within the range of either reference expression levels, enabling categorizing the test sample as coming from either a non-responding or responding patient.

Alternatively, and not mutually exclusive (both can be optionally combined), the invention relates to methods for determining or predicting (clinical) response of a cancer patient to immunotherapy or to therapy comprising an immunotherapeutic compound or agent, comprising:
determining, assessing, measuring, or quantifying the number of mesenchymal-like cancer (MES) cells in a sample obtained from a cancer lesion, wherein the sample is obtained after the patient having cancer received at least one administration of the immunotherapy or of the therapy comprising an immunotherapeutic compound or agent;
determining or predicting the cancer patient having cancer to (clinically) respond to the immune checkpoint inhibitor therapy or therapy comprising the immune checkpoint inhibitor when the number of mesenchymal-like cancer cells in the sample corresponds to the number of mesenchymal-like cancer cells in the same type of cancer of patients known to (clinically) respond to the immunotherapy or to the therapy comprising an immunotherapeutic compound or agent.

The number of MES cells in the same type of cancer of patients known to (clinically) respond to the immunotherapy or to the therapy comprising an immunotherapeutic compound or agent herein form the reference number of MES cells, in particular the responder reference number of MES cells, to which a test sample's number of MES cells are compared.

The number of MES cells in the same type of cancer of patients known not to (clinically) respond to the immunotherapy or to the therapy comprising an immunotherapeutic compound or agent in general will also be available, and form another set of reference number of MES cells, in particular the non-responder reference number of MES cells.

Non-responder reference number of MES cells and responder reference number of MES cells each can be expressed as minimum to maximum number, as average numbers, or as average+/−standard deviation numbers; and each are typically derived from a plurality of treated patients for which the (clinical) response has been determined/is known.

Non-responder reference number of MES cells and responder reference number of MES cells are numerically distinguishable from each other and a test sample's number of MES cells will “correspond” to either one, i.e., will either be close to either reference number or will be within the range of either reference number, enabling categorizing the test sample as coming from either a non-responding or responding patient.

These methods for determining or predicting (clinical) response of a cancer patient to immunotherapy or to therapy comprising an immunotherapeutic compound or agent can optionally be extended and can further comprise:
determining, assessing, measuring, or quantifying the number of antigen-presenting cells in a sample obtained from a cancer lesion, wherein the sample is obtained before the patient having cancer received at least one administration of the immunotherapy or of the therapy comprising an immunotherapeutic compound or agent;
determining or predicting the cancer patient having cancer to (clinically) respond to the immunotherapy or to the therapy comprising an immunotherapeutic compound or agent when the number of antigen-presenting cells in the sample correspond to the number of antigen-presenting cells in the same type of cancer of patients known to (clinically) respond to the immunotherapy or to the therapy comprising an immunotherapeutic compound or agent.

Reference numbers of antigen-presenting cells in (non-) responders to immunotherapy or therapy comprising an immunotherapeutic compound or agent can be defined as outlined hereinabove for MES cells.

In one embodiment, the methods are in part computer-implemented methods.

### Identification of Mesenchymal-Like Cells in a Cancer or Tumor

In a further aspect, the invention relates to methods of determining the presence of mesenchymal-like (MES) cancer cells in a tumor, comprising:
assessing the presence of a protein and/or RNA encoding the protein in a sample obtained from the tumor, wherein the protein and/or RNA is a product of a gene of Table 1 or 3 (MES-genes; see Example 2 hereinafter);
assessing the presence of a protein and/or RNA encoding the protein in a sample obtained from the tumor, wherein the protein and/or RNA is a product of a gene of Table 4 or 5 (minimal lineage gene signature; see Example 4 hereinafter);
assessing the presence of a protein and/or RNA encoding the protein in a sample obtained from the tumor, wherein the protein and/or RNA is a product of a cancer cell gene;
determining mesenchymal-like cancer cells to be present in a tumor when detecting cancer cells in which a product of a gene of Table 1 or 3, a product of a gene of Table 4 or 5, and a product of a cancer cell gene are present.

In such methods, a protein can be detected by immunohistochemistry, multiplex immunohistochemistry, or by spatial proteomics.

In such methods, an RNA can be detected in silico or in situ, or can be detected by in situ hybridization, fluorescent in situ hybridization, spatial transcriptomics, multiplex in situ hybridization, or can be detected in bulk transcriptomic data.

In a further aspect, the invention relates to methods of determining the presence of mesenchymal-like cancer cells in a tumor, comprising detecting co-localization of a product of a gene of Table 1 or 3, a product of a gene of Table 4 or 5, and a product of a cancer cell gene in the same cancer cell in a sample obtained from a patient having cancer, and designating such cancer cell as mesenchymal-like cancer cell.

In one embodiment to the methods of determining the presence of mesenchymal-like cancer cells in a tumor, the gene of Table 1 or 3 is THY1, LUM, DCN, and/or TCF. In a further embodiment, the gene of Table 4 or 5 is CDH19, S100A1, MITF and/or SOX10. In a further embodiment, the cancer cell gene is MITF and/or SOX10, and/or is gene with a mutation specific to the cancer cell. In a further embodiment, the cancer is melanoma or metastatic melanoma. In a further embodiment, the methods are in part computer-implemented methods.

Genes with a mutation specific to a cancer cell are known in the art and include e.g. the BRAF^{V600E} gene mutation.

In cases wherein in addition the antigen-presenting cells are to be assessed, methods similar as for determining MES-cells can be employed. Suitable proteins and/or RNAs encoding such proteins are proteins/genes such as listed in Table 2 (see Example 2 hereinafter).

### Spatial Proteomics

A good overview of spatial detection methods is provided by Lewis et al. 2021 (Nature Methods 18:997-1012) with FIGS. 3 to 5 therein summarizing the described methods and their performance.

The most classical spatial detection methods are histopathological staining (e.g. heamotxylin and eosin staining) and immunohistochemical staining. Histopathological staining provides information on different tissue structures and possible abnormalities therein. Immunohistochemical (IHC) staining involves binding of antibodies to target proteins of interest, usually these (primary) antibodies are unlabeled and (primary) antibodies bound to its target in e.g. a tissue section are subsequently detected by binding of a labeled, e.g. fluorescently labeled, (secondary) antibody that binds to the (primary) antibody bound to the target protein of interest. In multiplexed IHC (mIHC) usually up to 4 or 5 target proteins of interest can be detected simultaneously. In modern mIHC, iterative cycles of target protein of interest detection are applied. This involves successive cycles of antibody binding and stripping of the antibody or stripping or bleaching of the antibody-labels. Alternatively, a pool of DNA-barcoded antibodies is applied and iterative hybridization with differently labeled oligonucleotides is performed.

As a result, some of these techniques can detect up to 100 different proteins can be detected in a single tissue sample.

Non-iterative methods of target protein of interest detection involve binding of metal isotope-labeled antibodies that are subsequently detected by mass spectrometry upon release from a sample by means of tissue ablation with a laser beam (IMC: imaging mass cytometry) or tissue ionization with an ion beam (MIBI: multiplexed ion beam imaging). These techniques can detect up to 40 different proteins can be detected in a single tissue sample. IMC also allows for detection of an RNA target of interest. Concurrent quantitation of more than 40 proteins of interest is furthermore possible by quantitative analysis (involving sequencing) of oligonucleotides cleaved off from oligo-nucleotide labeled primary antibodies, this in a technique called digital spatial profiling (DSP). DSP also allows for detection of an RNA target of interest. These spatial proteomic techniques have been summarized in e.g. FIG. 3 of Lewis et al. 2021 (Nature Methods 18:997-1012).

### Spatial Transcriptomics

Spatial transcriptomics rely on direct detection of transcripts of interest with fluorescently labeled probes, the technology called fluorescent in situ hybridization (FISH). Many different FISH-based spatial transcriptomic methods have been developed and include iterative hybridization or bleaching or destruction of the labeled probes. Some of the FISH-based spatial transcriptomic methods allow for detection of up to 10000 different transcripts (summarized in e.g. FIG. 4 of Lewis et al. 2021 (Nature Methods 18:997-1012). Another series of spatial transcriptomic methods involve sequencing. In vitro methods (e.g. on a tissue sample) include laser capture microdissection (LCM) methods, methods including an mRNA capture step, and microfluidic-based methods-all have been reported to allow for detection of 10000 or more targets. In situ methods include in situ sequencing and fluorescence in situ sequencing methods. The sequencing technique can rely on sequence-by-ligation or sequence-by-hybridization methodologies. Again, some of these methods have been reported to allow for detection of 10000 or more targets (incompletely summarized in e.g. FIG. 5 of Lewis et al. 2021 (Nature Methods 18:997-1012).

### Sample/Cancer Lesion

A “sample” as referred to herein in general is a sample obtained from a tissue affected by a tumor or cancer (also referred to as “cancer lesion”). The tissue affected by a tumor or cancer/the cancer lesion may be the tumor or cancer itself, but can also refer to e.g. a lymph node metastasis in case of a metastatic tumor or cancer. The sample can be obtained as a or from a biopsy. The sample comprises at least one cancer or tumor cell. The sample furthermore may furthermore be a section of a fixed or frozen biopsy.

### Diagnostic Kits

The invention further relates to (therapeutic) kits comprising one or more containers or vials (any suitable container or vial, such as a pharmaceutically acceptable container or vial) comprising a: labeled; antibody or labeled oligonucleotide to detect a product of a gene of Table 1 or 3, a product of a gene of Table 4 or 5, a product of a cancer cell gene, and/or a product of a gene of Table 2. In particular the (labeled) antibody or labeled oligonucleotide is in one embodiment adapted for multiplex in situ analysis or detection (see sections on spatial proteomics/transcriptomics).

Further in particular, the kit is a kit for use in performing the above-described methods of determining response to immunotherapy; or for use in performing the above-described methods of identification of mesenchymal-like cells in a cancer or tumor.

Other optional components of such kit include one or more (further) diagnostic agents capable of predicting, prognosing, or determining the success of a therapy comprising one of the therapies according to the invention; use instructions; one or more containers with sterile pharmaceutically acceptable carriers, excipients or diluents [such as for producing or formulating a (pharmaceutically acceptable) composition of the invention]; one or more syringes; one or more needles; etc. In particular, such kits may be diagnostic kits or companion diagnostic kits.

### Gene Expression and Quantification of Gene Expression

The term “level of expression” or “expression level” generally refers to the amount of an expressed biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) are also regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs, long non-coding RNA, microRNA or miRNA).

“Increased/higher expression,” “increased/higher expression level,” “increased/higher levels,” “elevated expression,” “elevated expression levels,” or “elevated levels” refers to an increased/higher expression or to increased/higher levels of a biomarker in an individual relative to a suitable control or standard.

The term “detection” includes any means of detecting, including direct and indirect detection. The term “biomarker” as used herein refers to an indicator molecule or set of molecules (e.g., predictive, diagnostic, and/or prognostic indicator), which can be detected in a sample. The biomarker may be a predictive biomarker and serve as an indicator of the likelihood of sensitivity or benefit to therapeutic treatment of a patient having a particular disease or disorder (e.g., a proliferative cell disorder (e.g., cancer)) to treatment. Biomarkers include, but are not limited to, polynucleotides (e.g., DNA and/or RNA (e.g., mRNA)), polynucleotide copy number alterations (e.g., DNA copy numbers), polypeptides, polypeptide and polynucleotide modifications (e.g., post-translational modifications, nucleotide substitutions, nucleotide insertions or deletions (indels)), carbohydrates, and/or glycolipid-based molecular markers. In some embodiments, a biomarker is a gene. The “amount” or “level” of a biomarker, as used herein, is a detectable level in a biological sample. These can be measured by methods known to one skilled in the art and also disclosed herein.

In first instance, methodologies for determining gene expression by means of determining transcript levels, also referred to as transcriptome analysis or analysis of the transcriptome, is described in more detail. Any such gene detection or gene expression detection method is starting from an analyte nucleic acid (i.e. the nucleic acid of interest (which does not necessarily need to be the whole nucleic acid of interest, parts of such nucleic acids can suffice for determining expression) and of which the amount is to be determined) and may be defined as comprising one or more steps of, for instance,
a step of isolating RNA from a (biological) sample (wherein a fraction of the isolated RNA is the analyte strand);
a step of reverse transcribing the RNA obtained from the biological sample into DNA;
a step of amplifying the isolated DNA; and/or
a step of quantifying the isolated RNA, the DNA obtained after reverse transcription, or the amplified DNA.

In case an amplified DNA is quantified, this quantification step can be performed concurrent with the amplification of the DNA, or is performed after the amplification of the DNA.

The quantification of gene expression or the determination of gene expression levels may be based on at least one of an amplification reaction, a sequencing reaction, a melting reaction, a hybridization reaction or a reverse hybridization reaction.

The invention covers methods which include detection/quantification of nucleic acids corresponding to one or more biomarkers as defined herein. In any of these methods the detection can comprise a step such as a nucleic acid amplification reaction, a nucleic acid sequencing reaction, a melting reaction, a hybridization reaction to a nucleic acid, or a reverse hybridization reaction to a nucleic acid, or a combination of such steps.

Often one or more artificial, man-made, or non-naturally occurring oligonucleotide is used in such method. In particular, such oligonucleotides can comprise besides ribonucleic acid monomers or deoxyribonucleic acid monomers: one or more modified nucleotide bases, one or more modified nucleotide sugars, one or more labelled nucleotides, one or more peptide nucleic acid monomers, one or more locked nucleic acid monomers, the backbone of such oligonucleotide can be modified, and/or non-glycosidic bonds may link two adjacent nucleotides. Such oligonucleotides may further comprise a modification for attachment to a solid support, e.g., an amine-, thiol-, 3-′propanolamine or acrydite-modification of the oligonucleotide, or may comprise the addition of a homopolymeric tail (for instance an oligo (dT)-tail added enzymatically via a terminal transferase enzyme or added synthetically) to the oligonucleotide. If said homopolymeric tail is positioned at the 3′-terminus of the oligonucleotide or if any other 3′-terminal modification preventing enzymatic extension is incorporated in the oligonucleotide, the priming capacity of the oligonucleotide can be decreased or abolished. Such oligonucleotides may also comprise a hairpin structure at either end. Terminal extension of such oligonucleotide may be useful for, e.g., specifically hybridizing with another nucleic acid molecule (e.g. when functioning as capture probe), and/or for facilitating attachment of said oligonucleotide to a solid support, and/or for modification of said tailed oligonucleotide by an enzyme, ribozyme or DNAzyme. Such oligonucleotides may be modified in order to detect (the levels of) a target nucleotide sequence and/or to facilitate in any way such detection. Such modifications include labelling with a single label, with two different labels (for instance two fluorophores or one fluorophore and one quencher), the attachment of a different ‘universal’ tail to two probes or primers hybridizing adjacent or in close proximity to each other with the target nucleotide sequence, the incorporation of a target-specific sequence in a hairpin oligonucleotide (for instance Molecular Beacon-type primer), the tailing of such a hairpin oligonucleotide with a ‘universal’ tail (for instance Sunrise-type probe and Amplifluor™-type primer). A special type of hairpin oligonucleotide incorporates in the hairpin a sequence capable of hybridizing to part of the newly amplified target DNA. Amplification of the hairpin is prevented by the incorporation of a blocking non-amplifiable monomer (such as hexethylene glycol). A fluorescent signal is generated after opening of the hairpin due to hybridization of the hairpin loop with the amplified target DNA. This type of hairpin oligonucleotide is known as scorpion primers (Whitcombe et al. 1999, Nat Biotechnol 17:804-807). Another special type of oligonucleotide is a padlock oligonucleotide (or circularizable, open circle, or C-oligonucleotide) that are used in RCA (rolling circle amplification). Such oligonucleotides may also comprise a 3′-terminal mismatching nucleotide and/or, optionally, a 3′-proximal mismatching nucleotide, which can be particularly useful for performing polymorphism-specific PCR and LCR (ligase chain reaction) or any modification of PCR or LCR. Such oligonucleotide may can comprise or consist of at least and/or comprise or consist of up to 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200 or more contiguous nucleotides.

The analyte nucleic acid, in particular the analyte nucleic acid of a biomarker of interest can be any type of nucleic acid, which will be dependent on the manipulation steps (such as isolation and/or purification and/or duplication, multiplication or amplification) applied to the nucleic acid of the gene of interest in the biological sample; as such it can be DNA, RNA, cDNA, may comprise modified nucleotides, or may be hybrids of DNA and/or RNA and/or modified nucleotides, and can be single- or double-stranded or may be a triplex-forming nucleic acid.

The artificial, man-made, non-naturally occurring oligonucleotide(s) as applied in the above detection methods can be probe(s) or a primer(s), or a combination of both.

A probe capable of specifically hybridizing with a target nucleic acid is an oligonucleotide mainly hybridizing to one specific nucleic acid sequence in a mixture of many different nucleic acid sequences.

Specific hybridization is meant to result, upon detection of the specifically formed hybrids, in a signal-to-noise ratio (wherein the signal represents specific hybridization and the noise represents unspecific hybridization) sufficiently high to enable unambiguous detection of said specific hybrids. In a specific case specific hybridization allows discrimination of up to a single nucleotide mismatch between the probe and the target nucleic acids. Conditions allowing specific hybridization generally are stringent but can obviously be varied depending on the complexity (size, GC-content, overall identity, etc.) of the probe(s) and/or target nucleic acid molecules. Specificity of a probe in hybridizing with a nucleic acid can be improved by introducing modified nucleotides in said probe.

A primer capable of directing specific amplification of a target nucleic acid is the at least one oligonucleotide in a nucleic acid amplification reaction mixture that is required to obtain specific amplification of a target nucleic acid. Nucleic acid amplification can be linear or exponential and can result in an amplified single nucleic acid of a single- or double-stranded nucleic acid or can result in both strands of a double-stranded nucleic acid. Specificity of a primer in directing amplification of a nucleic acid can be improved by introducing modified nucleotides in said primer. The fact that a primer does not have to match exactly with the corresponding template or target sequence to warrant specific amplification of said template or target sequence is amply documented in literature (for instance: Kwok et al. 1990, Nucl Acids Res 18:999-1005. Primers as short as 8 nucleotides in length have been applied successfully in directing specific amplification of a target nucleic acid molecule (e.g. Majzoub et al. 1983, J Biol Chem 258:14061-14064).

A nucleotide is meant to include any naturally occurring nucleotide as well as any modified nucleotide wherein said modification can occur in the structure of the nucleotide base (modification relative to A, T, G, C, or U) and/or in the structure of the nucleotide sugar (modification relative to ribose or deoxyribose). Any of the modifications can be introduced in a nucleic acid or oligonucleotide to increase/decrease stability and/or reactivity of the nucleic acid or oligonucleotide and/or for other purposes such as labelling of the nucleic acid or oligonucleotide. Modified nucleotides include phosphorothioates, alkylphosphorothioates, methylphosphonate, phosphoramidate, peptide nucleic acid monomers and locked nucleic acid monomers, cyclic nucleotides, and labelled nucleotides (i.e. nucleotides conjugated to a label which can be isotopic (<32>P, <35>S, etc.) or non-isotopic (biotin, digoxigenin, phosphorescent labels, fluorescent labels, fluorescence quenching moiety, etc.)). Other modifications are described higher (see description on oligonucleotides).

Nucleotide acid amplification is meant to include all methods resulting in multiplication of the number of a target nucleic acid. Nucleotide sequence amplification methods include the polymerase chain reaction (PCR; DNA amplification), strand displacement amplification (SDA; DNA amplification), transcription-based amplification system (TAS; RNA amplification), self-sustained sequence replication (3SR; RNA amplification), nucleic acid sequence-based amplification (NASBA; RNA amplification), transcription-mediated amplification (TMA; RNA amplification), Qbeta-replicase-mediated amplification and run-off transcription. During amplification, the amplified products can be conveniently labeled either using labeled primers or by incorporating labeled nucleotides.

The most widely spread nucleotide sequence amplification technique is PCR. The target DNA is exponentially amplified. Many methods rely on PCR including AFLP (amplified fragment length polymorphism), IRS-PCR (interspersed repetitive sequence PCR), iPCR (inverse PCR), RAPD (rapid amplification of polymorphic DNA), RT-PCR (reverse transcription PCR) and real-time PCR. RT-PCR can be performed with a single thermostable enzyme having both reverse transcriptase and DNA polymerase activity (Myers et al. 1991, Biochem 30:7661-7666). Alternatively, a single tube-reaction with two enzymes (reverse transcriptase and thermostable DNA polymerase) is possible (Cusi et al. 1994, Biotechniques 17:1034-1036).

Solid phases, solid matrices or solid supports on which molecules, e.g., nucleic acids, analyte nucleic acids and/or oligonucleotides as described hereinabove, may be bound (or captured, absorbed, adsorbed, linked, coated, immobilized; covalently or non-covalently) comprise beads or the wells or cups of microtiter plates, or may be in other forms, such as solid or hollow rods or pipettes, particles, e.g., from 0.1 μm to 5 mm in diameter (e.g. “latex” particles, protein particles, or any other synthetic or natural particulate material), microspheres or beads (e.g. protein A beads, magnetic beads). A solid phase may be of a plastic or polymeric material such as nitrocellulose, polyvinyl chloride, polystyrene, polyamide, polyvinylidene fluoride or other synthetic polymers. Other solid phases include membranes, sheets, strips, films and coatings of any porous, fibrous or bibulous material such as nylon, polyvinyl chloride or another synthetic polymer, a natural polymer (or a derivative thereof) such as cellulose (or a derivative thereof such as cellulose acetate or nitrocellulose). Fibers or slides of glass, fused silica or quartz are other examples of solid supports. Paper, e.g., diazotized paper may also be applied as solid phase. Clearly, molecules such as nucleic acids, analyte nucleic acids and/or oligonucleotides as described hereinabove, may be bound, captured, absorbed, adsorbed, linked or coated to any solid phase suitable for use in hybridization assay (irrespective of the format, for instance capture assay, reverse hybridization assay, or dynamic allele-specific hybridization (DASH)). Said molecules, such as nucleic acids, analyte nucleic acids and/or oligonucleotides as described hereinabove, can be present on a solid phase in defined zones such as spots or lines. Such solid phases may be incorporated in a component such as a cartridge of e.g. an assay device. Any of the solid phases described above can be developed, e.g. automatically developed in an assay device.

Quantification of amplified DNA can be performed concurrent with or during the amplification. Techniques include real-time PCR or (semi-) quantitative polymerase chain reaction (qPCR). One common method includes measurement of a non-sequence specific fluorescent dye (e.g. SYBR Green) intercalating in any double-stranded DNA. Quantification of multiple amplicons with different melting points can be followed simultaneously by means of following or analyzing the melting reaction (melting curve analysis or melt curve analysis; which can be performed at high resolution, see, e.g. Wittwer et al. 2003, Clin Chem 843-860; an alternative method is denaturing gel gradient electrophoresis, DGGE; both methods were compared in e.g. Tindall et al. 2009, Hum Mutat 30:857-859).

Another common method includes measurement of sequence-specific labelled probe bound to its complementary sequence; such probe also carries a quencher and the label is only measurable upon exonucleolytic release from the probe (hydrolysis probes such as TaqMan probes) or upon hybridization with the target sequence (hairpin probes such as molecular beacons which carry an internally quenched fluorophore whose fluorescence is restored upon unfolding the hairpin). This latter method allows for multiplexing by e.g. using mixtures of probes each tagged with a different label e.g. fluorescing at a different wavelength.

Exciton-controlled hybridization-sensitive fluorescent oligonucleotide (ECHO) probes also allow for multiplexing. The hybridization-sensitive fluorescence emission of ECHO probes and the further modification of probes have made possible multicolor RNA imaging in living cells and facile detection of gene polymorphisms (Okamoto 2011, Chem Soc Rev, 40:5815-5828).

Other methods of quantifying expression include SAGE (Serial Analysis of Gene Expression) and MPSS (Massively Parallel Signature Sequencing), each involving reverse-transcription of RNA.

With “assaying” or “determining” or “detecting” and the like (e.g. assessing, measuring) is meant that a biological sample, suspected of comprising a target nucleic acid (such as a nucleic acid of a biomarker of interest as described herein), is processed as to generate a readable signal in case the target nucleic acid is actually present in the biological sample. Such processing may include, as described above, a step of producing an analyte nucleic acid. Simple detection of a produced readable signal indicates the presence of a target or analyte nucleic acid in the biological sample. When in addition the amplitude of the produced readable signal is determined, this allows for quantification of levels of a target or analyte nucleic acid as present in a biological sample.

In particular, the readable signal may be a signal-to-noise ratio (wherein the signal represents specific detection and the noise represents unspecific detection) of an assay optimized to yield signal-to-noise ratios sufficiently high to enable unambiguous detection and/or quantification of the target nucleic acid. The noise signal, or background signal, can be determined e.g. on biological samples not comprising the target or analyte nucleic acid of interest, e.g. control samples, or comprising the required reference level of the target or analyte nucleic acid of interest, e.g. reference samples. Such noise or background signal may also serve as comparator value for determining an increase or decrease of the level of a target or analyte nucleic acid in the biological sample, e.g. in a biological sample taken from a subject suffering from a disease or disorder, further e.g. before start of a treatment and during treatment.

The readable signal may be produced with all required components in solution or may be produced with some of the required components in solution and some bound to a solid support. Said signals include, e.g., fluorescent signals, (chemi) luminescent signals, phosphorescence signals, radiation signals, light or color signals, optical density signals, hybridization signals, mass spectrometric signals, spectrometric signals, chromatographic signals, electric signals, electronic signals, electrophoretic signals, real-time PCR signals, PCR signals, LCR signals, Invader-assay signals, sequencing signals (by any method such as Sanger dideoxy sequencing, pyrosequencing, 454 sequencing, single-base extension sequencing, sequencing by ligation, sequencing by synthesis, “next-generation” sequencing (NGS) (van Dijk et al. 2014, Trends Genet 30:418-426)), nanopore sequencing, melting curve signals etc. An assay may be run automatically or semi-automatically in an assay device. In view of its relatively low costs compared to e.g. very costly cancer therapies, NGS is finding its way to routine clinical care (Ratner 2018, Nature Biotechnol 36:484).

Specific hybridization of an oligonucleotide (whether or not comprising one or more modified nucleotides) to its target sequence is to be understood to occur under stringent conditions as generally known in the art (e.g. Sambrook et al. 1989. Molecular Cloning. A laboratory manual. CSHL Press). However, depending to the hybridization solution (SSC, SSPE, etc.), oligonucleotides should be hybridized at their appropriate temperature in order to attain sufficient specificity. In order to allow hybridization to occur, the target nucleic acid molecules are generally thermally, chemically (e.g. by NaOH) or electrochemically denatured to melt a double strand into two single strands and/or to remove hairpins or other secondary structures from single stranded nucleic acids. The stringency of hybridization is influenced by conditions such as temperature, salt concentration and hybridization buffer composition. High stringency conditions for hybridization include high temperature and/or low salt concentration (salts include NaCl and Na3-citrate) and/or the inclusion of formamide in the hybridization buffer and/or lowering the concentration of compounds such as SDS (detergent) in the hybridization buffer and/or exclusion of compounds such as dextran sulfate or polyethylene glycol (promoting molecular crowding) from the hybridization buffer. Conventional hybridization conditions are described in e.g. Sambrook et al. 1989 (Molecular Cloning. A laboratory manual. CSHL Press) but the skilled craftsman will appreciate that numerous different hybridization conditions can be designed in function of the known or the expected homology and/or length of the nucleic acid sequence. Generally, for hybridizations with DNA oligonucleotides without formamide, a temperature of 68 DEG C, and for hybridization with formamide, 50% (v/v), a temperature of 42 DEG C is recommended. For hybridizations with oligonucleotides, the optimal conditions (formamide concentration and/or temperature) depend on the length and base composition of the probe and must be determined individually. In general, optimal hybridization for oligonucleotides of about 10 to 50 bases in length occurs approximately 5 DEG C below the melting temperature for a given duplex. Incubation at temperatures below the optimum may allow mismatched sequences to hybridize and can therefor result in reduced specificity. When using RNA oligonucleotides with formamide (50% v/v) it is recommend to use a hybridization temperature of 68 DEG C for detection of target RNA and of 50 DEG C for detection of target DNA. Alternatively, a high SDS hybridization solution can be utilized (Church et al. 1984, Proc Natl Acad Sci USA 81:1991-1995). The specificity of hybridization can furthermore be ensured through the presence of a crosslinking moiety on the oligonucleotide (e.g. Huan et al. 2000, Biotechniques 28:254-255; WO00/14281). Said crosslinking moiety enables covalent linking of the oligonucleotide with the target nucleotide sequence and hence allows stringent washing conditions. Such a crosslinking oligonucleotide can furthermore comprise another label suitable for detection/quantification of the oligonucleotide hybridized to the target.

RPKM (Reads Per Kilobase Million) is often used as measure for expression. FPKM (Fragments Per Kilobase Million) is very similar to RPKM; whereas RPKM was designed for single-end RNA-seq (every read corresponded to a single sequenced fragment), FPKM was designed for paired-end RNA-seq. With paired-end RNA-seq, two reads can correspond to a single fragment, or, if one read in the pair did not map, one read can correspond to a single fragment. The only difference between RPKM and FPKM is that FPKM takes into account that two reads can map to one fragment (and so it doesn't count this fragment twice). When using RNA-seq, reporting or results often is in RPKM (Reads Per Kilobase Million) or FPKM (Fragments Per Kilobase Million). Whatever metric used (another alternative for example is TPM (Transcripts Per Kilobase Million)), such metric is attempting to normalize for sequencing depth and gene length and provide a measure for quantifying transcript levels/gene expression/expression units.

Next to methodologies for determining gene expression by means of determining transcript levels (transcriptome analysis), it is also possible to quantify gene expression by means of proteomic analysis (proteome analysis or analysis of the proteome). Classical proteomic analysis methods include ELISA, western blotting, mass spectrometry, chromatographic separation, immunohistochemistry, cell sorting (based on cell surface marker(s)) etc. Although not necessarily required, it can be advantageous to rely on multiplexed cytometry methods that can be performed directly on, e.g., a section of a breast cancer tissue biopsy (Formalin-Fixed Paraffin-Embedded (FFPE), fresh frozen (FF), . . . ). Multiplexed cytometry methods, as well as some predictive cancer biomarkers identified using such methodology, have been reviewed by e.g. Fan et al. 2020 (Cancer Communications 40:135-153) and have emerged with the advent of more sophisticated imaging techniques (e.g. cyclic immunofluorescence, tyramide-based immunofluorescence, epitope-targeted mass spectrometry, RNA detection) and standardized quantification methodologies. Such multiplexed cytometry methods include multiplex immunocytochemistry (mICH), imaging mass spectrometry, multiplexed ion beam imaging, chip cytometry, nucleotide (DNA/RNA)-barcoding-based mICH, and digital spacing profiling. Another technique involving proteomic analysis is Cellular Indexing of Transcriptomes and Epitopes by Sequencing (CITE-seq).

### Computer/Computer System

A computer or computer system as mentioned herein may utilize one or more subsystems. A computer or computer system may be a single computer apparatus comprising the one or more subsystems (e.g. internal components), or may be multiple computers or multiple computer apparatuses each being a subsystem, and optionally, each comprising one or more own subsystems. Desktops, laptops, mainframe servers, tablets, mobile phones etc. all are computers or computer systems. The subsystems are usually interconnected and include a (central) processor (single-core processor, multi-core processor on a same integrated chip, or multiple processing units on a single circuit board or networked) capable of executing instructions, an input/output (I/O) controller, and a storage device (external, internal, peripheral, cloud, any medium readable by a computer or computer system). Input devices include keyboards, scanners, a computer mouse, camera, microphone, etc. In particular, the input device is a data collection or data generating device (which by itself may comprise a computer or computer system), such as a polynucleotide sequencing device (whether automated or not). Collected or generated data are fed to a computer or computer system designed to analyze the collected or generated data; this may be an ordinary computer system on which data analyzing software is installed (on a storage device) or which is capable of accessing data analyzing software (e.g. installed in or transmitted from a network) and whereby the processor of the computer system is instructed by the data analysis software on how to process the collected or generated data fed to the computer system, and how to display these via a display adapter to an output device. Output devices are further subsystems and comprise printers, monitors, computer readable medium. Input and output devices are usually connected to a computer or computer system via input/output ports to one another or via a network.

The specific combination of hardware and software allows implementation of e.g. analysis of data generated by a polynucleotide sequencing device or expression analysis device, or analysis of data generated by an imaging device. Different software packages (proprietary or open source) can be run on a computer or computer system to achieve the desired degree of data analysis. Output of one computerized data analysis can be the input of a subsequent computerized data analysis step, hence creating an analysis pipeline. Software components can be written in different codes (e.g. Java, C, C++, Perl, Python) as long as the computer processor is able to execute the functions of the software component.

The methods of the invention may be computer-implemented methods, or methods that are assisted or supported (in part) by a computer or by a computer system. For instance, information reflecting the analysis, determination, detection, presence or absence of labeled markers in an image, or of determining, detecting, assaying, assessing or analyzing biomarker expression or biomarker expression levels obtained from a sample is received by at least one first processor, and/or information reflecting the analysis, determination, detection, presence or absence of labeled markers in an image, or of determining, detecting, assaying, assessing or analyzing biomarker expression or biomarker expression levels obtained from a sample is provided in user readable format by at least one/another processor.

The same or a further processor may be calculating relative labeled marker levels in an image (such as relative to a control or standard), or a relative biomarker expression or biomarker expression level (such as relative to a control or standard) from the information received. The one or more processors may be coupled to random access memory operating under control of or in conjunction with a computer operating system. The processors may be included in one or more servers, clusters, or other computers or hardware resources, or may be implemented using cloud-based resources. The operating system may be, for example, a distribution of the Linux™ operating system, the Unix™ operating system, or other open-source or proprietary operating system or platform. Processors may communicate with data storage devices, such as a database stored on a hard drive or drive array, to access or store program instructions other data. Processors may further communicate via a network interface, which in turn may communicate via the one or more networks, such as the Internet or other public or private networks, such that a query or other request may be received from a client, or other device or service. Such computer-implemented methods (or such methods that are assisted or supported by a computer) may be provided as a kit or as part of a kit. The bioinformatics software required to perform (part of) the computer-implemented methods, i.e. a computer program product, may also be part of a kit, or may be provided as an individual product. A computer product may also consist of a computer readable medium which is storing any of the instructions, computer program, or bioinformatics software enabling a computer system to perform at least one of the analysis of the herein described methods and/or to perform at least one calculation (e.g. of image analysis or of biomarker expression or biomarker expression level) as described herein.

### Other Definitions

The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, New York (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

In referring to genes or proteins herein, no distinction is made in the annotation. Thus, whereas for example the human TCF4/ITF2 gene would be referred to as the TCF4/ITF2 gene, the mRNA as TCF4/ITF2 mRNA, and the protein as TCF4/ITF2, such distinction is not, or not always, made hereinabove or hereinafter.

In any of the above, a “patient” in general is a mammalian species having cancer or a tumor or diagnosed with cancer. The mammalian species in general is a higher species including primates, cattle (e.g. cows, sheep, goats, pigs), horses, and pets (e.g. dogs, cats). In one embodiment the patient is a human subject.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims The content of the documents cited herein are incorporated by reference.

### EXAMPLES

### Example 1. Materials and Methods

### 1.1. Patient Biopsies

Tumor biopsies from were collected as part of a non-interventional prospective study investigating transcriptomic changes upon immune checkpoint inhibition (Prospective Serial biopsy collection before and during immune-checkpoint inhibitor therapy in patients with malignant melanoma; SPECIAL). While most patients (n=20) were treatment-naïve, patients with metastatic relapse were allowed prior systemic treatment in adjuvant setting (n=2). In addition, one patient had received Cisplatinum-based neo-adjuvant chemotherapy for a metachronous non-small cell lung carcinoma eight months before inclusion. Written informed consent was obtained from all patients. All study procedures were in accordance with the principles of the Declaration of Helsinki, applicable Belgian law and regulations, and approved by the UZ Leuven Medical Ethical Committee (S62275).

### 1.2. Tumor Dissociation of Human Samples

Fresh tumor tissue was collected in cold transport Dulbecco's Modified Eagle Medium (DMEM, Invitrogen, Cat #61965025) on ice. To make a single cell solution, tumor fragments were rinsed in cold Dulbecco's Phospho-Buffered Saline (DPBS) and mechanically and enzymatically dissociated. The tumor was minced with sterile scalpels and incubated 15 minutes in a heather-shaker at 37° C. 800 rpm in 1.32 mL DMEM supplemented with 120 μL DNase | (10 mg/ml; Sigma-Aldrich, Cat #11284932001) and 60 μL collagenase P (50 ng/ml; Sigma-Aldrich, Cat #11249002001). The sample was diluted 1:2 in DPBS, centrifuged 5 min. at 300G at room temperature, incubated 5 min. in 500 μL red blood lysis buffer at room temperature, washed twice with DPBS supplemented with 0.04% bovine serum albumin and strained through a 35 μm nylon mesh. Cell concentration and viability was determined with acridine orange/propidium iodide staining (Westburg, Cat #LB F23001) on a LUNA-FL automated fluorescence counter.

### 1.3. Single Cell RNA Sequencing

Libraries for scRNA-seq were constructed using the 10× Genomics Chromium platform according to manufacturer's instructions. Library construction was primarily done with the Chromium Single Cell 3′ GEM, Library & Gel Bead Kit v3 (10× genomics, Cat #1000092). Thirteen samples were processed using the Chromium Single Cell A Chip Kit and 5′ Library & Gel Bead Kit (10× genomics, Cat #1000014). When comparing sequenced 3′ and 5′ gene expression libraries from the same tumor samples, we observed similar quality metrics. We opted for high target recovery (median 5000, range 5000-10000), keeping within the range of optimal input concentration per target recovery, as recommended by the manufacturer. In brief, cells were partitioned into Gel Bead-in-emulsions (GEMs) at limiting dilution, where lysis and reverse transcription occurred yielding uniquely barcoded full-length cDNA from poly-adenylated mRNA. GEMs were subsequently broken, and the pooled fraction was amplified, followed by fragmentation, end repair and adaptor ligation of size selected fractions.

All libraries were sequenced with single end reads on an Illumina NextSeq, HiSeq4000 or NovaSeq6000 until sufficient saturation was reached (60% on average). The raw sequencing reads were processed by CellRanger (10× Genomics), human reference genome v. GRCh38).

### 1.4. ScRNA-Seq Data Analysis

Raw count matrices were analysed using R package Seurat v. 3.1.541. The matrices were filtered by removing cell barcodes with >1000 expressed genes, <7,500 expressed genes and <30% of reads mapping to mitochondrial reads. Next, SCTransform was applied to each Seurat object for data normalization and transformation. DoubletFinder v. 2.0.283 was applied to each Seurat object (sample) separately assuming that the doublet rate in each sample was as indicated in the 10× Genomics website. Next, all the Seurat objects were merged, SCTransform was applied regressing out mitochondrial read percentage per cell. Subsequently, the data integration was performed using R package Harmony v. 1.040. After having normalized and integrated the data, cell cycle scoring was performed, data were filtered for singlets, and SCTransform was applied regressing for mitochondrial read percentage and cell cycle scores. This was followed by data integration of this subset as described above. The number of dimensions for clustering were chosen based on Harmony embeddings clustering. The cut-off was driven by identification of clear variation in embeddings across the cells. For cell type identification from the malignant, immune and stromal compartments we analysed the data including cells from both time points, but for the detailed characterisation of the treatment naïve samples we subset only for this time point.

### 1.5. Initial Identification of the Tumor Microenvironment Compartments

To gain a global view on the components of the tumor microenvironment we used existing signatures acquired from Jerby-Arnon et al. 2018 (Cell 175:984-997.e24) to calculate gene set scores using R package AUCell v.1.6.145 for the immune, stromal and malignant compartments. By plotting the scores, we assigned each unsupervised cluster to one of these three compartments.

1.6. CNV Inference in human samples

To distinguish malignant from normal cells we inferred copy number variation (CNV) based on scRNA-seq data using the R package HoneyBadger v. 0.146. The count matrix from the “RNA” assay of the integrated Seurat object of all cells was used as input. Immune cells were used as a reference for normal cells. They were defined based on the immune gene set from Jerby-Arnon et al. 2018 (Cell 175:984-997.e24), using an AUCell score cut-off >0.15. The mean CNV score was calculated as below:

$$CNV score = \frac{{\sum }_{i} \vert {G}_{cnv , i} \vert}{n}$$

where, G=gene, i=cell.

### 1.7. Identification and Analysis Malignant Cells.

Differential gene expression was run between globally classified malignant clusters (2,28,0,12,17,20,19) vs CAFs clusters (7,8) using Seurat FindMarkers function (Wilcoxon Rank Sum test). Next, for each gene, the difference of the percentage of cells expressing this gene in the malignant clusters minus the CAFs clusters was calculated and the genes were sorted in descending order. The top 50 genes were plotted on the global UMAP in order to identify the most specific and ubiquitously expressed ones within malignant clusters further called as Melanoma Score (MS).

To identify malignant cells, three stringent steps of filtration were applied. Firstly, the data was subsetted based on the AUCell score of malignant gene set acquired from Jerby-Arnon et al. 2018 (Cell 175:984-997.e24)>0.11 or mean CNV score >0.15. Subsequently, cells that passed the first filtration step were filtered based on the MS >0.2 or mean CNV score >0.15. Finally, to remove any contaminating immune cells, we filtered out PTPRC (CD45) cells. Lastly, samples with less than 10 remaining cells were removed from downstream analysis.

The subsets of malignant cells were resubjected to SCTransform (regressing out mitochondrial read percentage and the cell cycle scores,) and Harmony integration (grouping the variables by samples) followed by unsupervised Seurat clustering. The number of dimensions for clustering were chosen based on Harmony embeddings clustering—a cut-off was driven by identification of clear variation in embeddings across the cells. Number of clusters was chosen based on Silhouette (Rousseeuw 1987, J Comput Appl Math 20:53-65) scores measured at different resolutions and biological relevance of the marker genes per cluster. The marker genes of each unsupervised and semi-supervised cluster were identified using FindAllMarkers function in Seurat (Wilcoxon Rank Sum test). The final cluster annotations were based on the enriched pathways and terms of the top marker genes per cluster (top 100 genes) using an online tool Enrichr (https://maayanlab.cloud/Enrichr/). To understand the biological identity of the malignant clusters, we used databases such as Gene Ontology (GO), Reactome, OMIM Disease, MSigDB Hallmark 2020, Jensen Compartments, CellMarker Augmented, and CCLE Proteomics.

### 1.8. Gene Regulatory Network Analysis

SCENIC (Bolognesi et al. 2017, J Histochem Cytochem 65:431-444) analysis was run with raw counts from the “SCT” assay of malignant cells 50×. SCENIC uses gene regulatory network inference, followed by a refinement step using cis-regulatory information, to generate a set of refined regulons (i.e. TFs and their target genes) in the ScRNA-seq data. The Python implementation, (pySCENIC: https://github.com/aertslab/pySCENIC, version 0.9.19), was run using a Nextflow pipeline (https://github.com/aertslab/SCENICprotocol, version 0.2.0), which streamlined the main steps of gene regulatory network inference and refinement with pySCENIC, as well as the quantification of cellular activity, and visualization. The Nextflow pipeline also performed a standard analysis in parallel, using highly variable genes selected based on expression. Differentially activated TF regulons of each malignant cluster were identified by the Wilcoxon rank sum test against all the cells of the rest of the clusters. To infer cell-cell interactions we used the Seurat object and run CellChat (Jin et al. 2021, Nat Commun 12:1088) version 1.1.3 applying 10% truncated mean for average gene expression per cell group and minimum twenty cells required per cell.

### 1.9. Identification of the Minimal Lineage Gene Signature (MLGs)

To identify the MLGs we subsetted CAFs together with the Mesenchymal-like state and performed differential gene expression analysis between them. The top 50 genes were called the MLGs, from which four (SOX10, S100A1, MITF and CDH19), were selected for further validation by Akoya/RNAscope.

1.10. Validation of Identified Melanoma States in Independent scRNA-Seq Dataset

Transcript per Million (TPM) normalized the Jerby-Arnon et al. 2018 (Cell 175:984-997.e24) dataset was downloaded from the GEO portal (Accession number GSE115978). The TPM normalized dataset was used to generate a Seurat object. The cells with a number of genes >1000 & <7500 were selected for further analysis. Next, we calculated the AUCell score for the SMS using the same set of genes as in the main cohort (EDNRB, MYO10, PLP1, ERBB3, SYNGR1). The malignant cells were subsetted based on an SMS score >0.1 and the criteria applied in the Jerby-Arnon et al. 2018 (Cell 175:984-997.e24) study. Additionally, cells positive for PTPRC were excluded. Next, the data was scaled regressing out the cell cycle scores and percentage of expressed mitochondrial genes, and integrated using Harmony.

To validate the transcriptomic states identified in our dataset we furthermore performed label transfer of the malignant clusters in Seurat, using the following parameters: integration features=3000, k·anchor=20, and reduction=“pcaproject”. The prediction scores were plotted on the Harmony integrated UMAP.

### 1.11. Spatial Transcriptomics

Selected samples were processed for spatial transcriptomics using the 10× Genomics Visium platform. The analysis of these was approved by the Ethical Commission of the University Hospital of Leuven and approved by the review board (#S55760). Tumors were dissected, washed with 1× DPBS and snap-frozen in liquid nitrogen-chilled isopentane. Frozen tumors were transferred to a cold tissue mould filled with chilled optimal cutting temperature compound (Tissue-Tek O.C.T. compound, Sakura Finetek Cat #4583). The mould was then immediately placed on dry ice. Tissue blocks were stored at −80° C. in a sealed container. Both the tissue block and the proprietary Visium Spatial Gene Expression Slide (10× Genomics, Cat #PN-2000233) were equilibrated inside the cryostat for 30 min at −12° C. before sectioning. Sections were cut at a thickness of 10 μm and immediately placed onto the slide. Slides containing sections were stored at −80° C. for 24 h before use.

Fixation, staining, imaging, and construction of cDNA libraries was done according to the manufacturer's instructions (Visium Spatial Gene Expression User Guide_Rev F) (10× Genomics, CG000239) using the Visium Spatial Gene Expression Slide & Reagent Kit (10× Genomics, Cat #PN-1000187). Briefly, sections were fixed in chilled methanol for 30 min at −20° C. and stained with hematoxylin and eosin. Imaging was performed on a Nikon-Marzhauser Slide Express 2 whole-slide scanner at 20× magnification. After imaging, sections were permeabilized at 37° C. for 20 minutes. Permeabilization time was determined using the Visium Spatial Tissue Optimization Slide & Reagent Kit (10× Genomics, PN-1000193) following the Visium Spatial Tissue Optimization User Guide_RevE (10× Genomics, CG000238). After permeabilization, the on-slide reverse transcription reaction was performed at 53° C. for 45 min. Second strand synthesis was subsequently performed on-slide for 15 min at 65° C. All on-slide reactions were performed in a thermocycler with a metal slide adapter plate. Following second strand synthesis, samples were transferred to tubes for cDNA amplification and clean-up. Cycle number determination for cDNA amplification was done by qPCR. Library QC was assessed using an Agilent Technologies 4150 TapeStation Systems using the High Sensitivity D5000 ScreenTape and DNA Analysis Reagents (Agilent Technologies, Cat #5067-5592 & Cat #5067-5593).

Visium libraries were sequenced on Illumina Novaseq 6000. Raw sequencing files were processed with SpaceRanger (v1.0.0, 10× Genomics) to generate spatial gene expression matrices. Next, the data was analysed using the Seurat spatial vignette.

Spots with spatial features >500 and percentage of mitochondrial reads either <3 or <5 were retained and the expression data was normalized using SCTransform (Seurat v3.2.3) (Korsunsky et al. 2019, Nat Methods 16:1289-1296). Firstly, the spatial distribution of the major TIME constituents such as malignant cells, T cells, B cells, macrophages, CAFs, and ECs was mapped using the label transfer function with CCA-based label transfer (k anchor=10). Furthermore, to identify true malignant spots, we leveraged copy number inference using HoneyBadger (Fan et al. 2018, Genome Res 28:1217-1227). B cell or ECs spots with prediction score >0.8 were used as “normal” reference. Subsequently, we selected spots with a prediction score >0.7 for the malignant label and the mean CNV score (calculated as described above)>0.07 and annotated these as melanoma. Finally, for the malignant cell deconvolution, distance and colocalization calculations we used the CellTrek (Wei et al. 2022, Nat Biotechnol doi: 10.1038/s41587-022-01233-1) R package.

### 1.12. Multiplex Immunostaining Followed by Multiplex FISH

Five μm FFPE tissue sections of selected samples were cut and mounted on poly-L-lysine coated coverslips. Akoya Biosciences CODEX multiplex immunostaining (CODEX) and ACDbio RNAscope HiPlex v2 (12-plex) multiplex FISH (RNAscope) were each performed according to their respective manufacturer's instructions, and combined in sequence as previously described, with slight modifications (Cheng et al. 2022, bioRxiv 2022.02.10.479971).

In brief, coverslips were deparaffinized followed by heat-induced antigen retrieval in citrate buffer, pH 6. Next, they were stained with a combination of DNA-barcoded primary antibodies, washed and post-fixed in ice-cold methanol. They were mounted on an Akoya Biosciences CODEX system for multiple cycle immunostaining and imaged using a Keyence microscope with Akoya Biosciences CODEX instrument manager and Keyence software. Secondary antibodies were fed to the instrument in a pre-prepared 96-well plate. In total, 11 cycles of immunostaining (including 2 blanks with only nuclear staining) were run, consisting of DAPI nuclear staining, Atto550-, Cy5- and Alexa Fluor 750 fluorophores. Akoya Biosciences CODEX processor software performed automated image registration, autofluorescence and background subtraction. Cover slips were kept in storage buffer until RNAscope was performed.

To prepare samples for RNAscope, samples were washed in ethanol for 2 min. and air dried for 5 min. in a 60° C. oven. Target retrieval was followed by protease treatment. The 12-plex RNAscope assay consisted of 3 rounds (each round using 4 probes) of probe hybridization, amplification, autofluorescence reduction, fluorophore hybridization, DAPI counterstaining, imaging, fluorophore cleavage and washing. Coverslips were imaged using VectraPolaris Automated Quantitative Pathology Imaging System. CODEX images were registered to RNAscope using the BigWarp plugin for ImageJ (Bogovic et al. 2016, In Proc Intl Symp Biomed Imaging June 2016, 1123-1126). The CODEX image stack was used as a fixed target to register the 3 RNAscope imaging rounds onto, using manually placed landmarks. This resulted in resampling of the RNAscope to target resolution. Next, regions of interest (ROIs) of 100×100 μm were delineated using QuPath Quantitative Pathology & Bioimage Analysis software (Bankhead et al. 2017, Sci Rep 7:16878). In these ROIs, the autofluorescence channel of each RNAscope imaging round was subtracted from each respective fluorescent channel using the Image Calculator in ImageJ. Cells were segmented with the StarDist (Schmidt et al. 2018, Cell Detection with Star-Convex Polygons BT-Medical Image Computing and Computer Assisted Intervention-MICCAI 2018. in (eds. Frangi, A. F., Schnabel, J. A., Davatzikos, C., Alberola-López, C. & Fichtinger, G.) 265-273 (Springer International Publishing, 2018)) extension in QuPath, using the dsb2018_heavy_augment.pb pretrained model (Caicedo et al. 2019, Nat Methods 16:1247-1253).

1.13. MILAN (mIHC)

Multiplex immunofluorescent staining was performed according to the previously published MILAN protocol (Bolognesi et al. 2017, J Histochem Cytochem 65:431-444). The antibody panel was designed to allow the identification of the most relevant cell types derived from the scRNA-seq results. Immunofluorescence images were scanned using the Axio scan.Z1 slidescanner (Zeiss, Germany) at 10× objective with resolution of 0.65 μm/pixel. All samples were stained simultaneously. Image acquisition order was distributed spatially and independently of patient replicates. The stains were visually evaluated for quality by digital image experts and experienced pathologists (FMB, YVH, double-blind). Multiple approaches were taken to ensure data. On the image level, focus, presence of external artifacts and tissue integrity were reviewed. Regions that contained severely deformed tissues and artifacts were identified and excluded from downstream analysis. Antibodies that gave low confidence staining patterns by visual evaluation were excluded from the analysis. Image analysis was performed following a custom pipeline. Briefly, flat field correction was performed using a custom implementation of a previously described algorithm (Kask et al. 2016, J Microsc 263:328-340). Then, adjacent tiles were stitched by minimizing the Frobenius distance of the overlapping regions. Next, images from consecutive rounds were registered following an algorithm previously described (Srinivasa Reddy et al. 1996, IEEE Trans Image Process 5:1266-1271). During this process, the first round was always used as a fixed image whereas all consecutive rounds were sequentially used as moving images. Transformation matrices were calculated using the DAPI channel and then applied to the rest of the channels. Registration results were visually inspected by domain experts (FMB, YVH). Samples with tissue folds showed significant misalignments and were manually segmented in different regions. Each region was independently re-registered. Downstream analysis was independently performed for each annotated region. Next, tissue autofluorescence was subtracted using a baseline image with only secondary antibody. Finally, cell segmentation was applied to the DAPI channel using StarDist (Schmidt et al. 2018, Cell Detection with Star-Convex Polygons BT-Medical Image Computing and Computer Assisted Intervention-MICCAI 2018. in (eds. Frangi, A. F., Schnabel, J. A., Davatzikos, C., Alberola-López, C. & Fichtinger, G.) 265-273 (Springer International Publishing, 2018)). For every cell, topological features (X/Y coordinates), morphological features (nuclear size), and molecular features (Mean Fluorescence Intensity (MFI) of each measured marker) were extracted.

### 1.14. Cell Identification

MFI values were normalized within each region to Z-scores as recommended in (Caicedo et al. 2019, Nat Methods 16:1247-1253). Zscores were trimmed in the [0, 5] range to avoid a strong influence of possible outliers in downstream analyses. Single cells were mapped to known cell phenotypes using three different clustering methods: PhenoGraph (Levine et al. 2015, Cell 162:184-197), FlowSom (Van Gassen et al. 2015, Cytom Part A 87:636-645), and KMeans as implemented in the Rphenograph, FlowSOM, and stats R packages. While FlowSom and KMeans require the number of clusters as input, PhenoGraph can be executed by defining exclusively the number of nearest neighbours to calculate the Jaccard coefficient 30. The number of clusters identified by PhenoGraph was then passed as an argument for FlowSom and KMeans. Clustering was performed exclusively in a subset of the identified cells (50,000) selected by stratified proportional random sampling and using only the 23 markers defined as phenotypic. For each clustering method, clusters were mapped to known cell phenotypes following manual annotation from domain experts (FMB, YVH, double-blind). If two or more clustering methods agreed on the assigned phenotype, the cell was annotated as such. If all three clustering methods disagreed on the assigned phenotype, the cell was annotated as “not otherwise specified” (NOS). Annotated cells were used to construct a template that was in turn used to extrapolate the cell labels to the rest of the dataset. To that end, a UMAP was built by sampling 500 cells for each identified cell type in the consensus clustering. The complete dataset was projected into the UMAP using the base predict R function. For each cell, the label of the closest 100 neighbours was evaluated in the UMAP space and the label of the most frequent cell type was assigned.

Melanoma cells were further segmented based on the expression of HLA-DR (Gadeyne et al. 2021, Front Oncol 11:636057). Here, we set a cut-off of Z=2 to differentiate between HLA-DR positive and HLA-DR negative melanoma cells.

### 1.15. Neighbourhood Analysis

For neighbourhood analysis, a quantitative analysis of cell-cell interactions was performed using an adaptation of the algorithm described in Schapiro et al. 2017 (Nat Methods 14:873-876). A detailed description of the adapted implementation was previously published (Bosisio et al. 2020, ELife 9: e53008). Briefly, for every cell, all the other cells that are located at a maximum distance d were counted. Then the tissue is randomized preserving the cytometry of the tissue as well as the X and Y coordinates of each cell but permutating the cell identities. This is repeated N times (here N=1000) which allows to assign an empirical p-value by comparing the number of counts observed in the real tissue versus the N random cases. We performed the described analysis for different values of the distance d (from 10 to 100 μm with a step of 10 μm) to show the consistency of the reported results. Particularly here, the analysis was performed exclusively to evaluate whether CD3+ and CD8+ T cells (Tcys) were interacting more with HLA-DR positive or HLA-DR negative melanoma cells. Therefore, we only included melanoma subtypes in the randomization process while keeping all the other cell subtypes unchanged. To add an effect-size metric, we also calculated the ratio between the observed counts and the random counts.

### 1.16. Cell Culture

The human melanoma cell cultures were derived from patient biopsies by the Laboratory of Oncology and Experimental Surgery (Prof. Dr. Ghanem Ghanem, Institute Jules Bordet, Brussels, Belgium). All cell lines (MM011, MM029, MM034, MM047, MM057, MM099, MM164) were grown in 5% CO2 at 37° C. in F10 supplemented with 10% FBS, 2.5% GlutaMAX and 1% penicilline/streptomycin. Jurkat T cells (human leukaemic T-cell line; Abraham & Weiss 2004, Nat Rev Immunol 4:301-308) were grown in DMEM with 10% FBS and 1% penicillin/streptomycin.

A375 and HEK293 FT cells (human embryonal kidney 293T cells transformed with the SV40 large T antigen) were grown in DMEM with 10% FBS and 1% penicillin/streptomycin.

Ma-Mel-86c human melanoma cells (Zhao et al. 2016, Cancer Res 76:4347-4358) were grown in RPMI with 10% FBS and 1% penicillin/streptomycin.

Cells were tested for Mycoplasma contamination prior to performed experiments.

### 1.17. Drugs

BRAFi Dabrafenib (Cat #HY-14660) and MEKi Trametinib (Cat #HY-10999) were purchased from MedChemExpress. ARV-771 (Cat #HY-100972) was purchased from Bioconnect.

1.18. siRNA-Mediated Transient Genetic Inactivation

Cells were transfected with the indicated specific short interfering RNA (siRNA) SMARTpools (Dharmacon, Cat #L-004594-00-0005 and Cat #D-001810 Oct. 20) using TransIT-X2 Transfection Reagent (Mirus) according to the manufacturer's protocol. siRNAs were used at a final concentration of 50 nM.

### 1.19. Lentiviral Vector Production

HEK293 FT cells were transfected with dVPR and VSVG packaging plasmids using Lipofectamine 2000 reagent (ThermoFisher Scientific) according to the manufacturer's instructions. 24 hours after transfection, medium was replaced with DMEM medium (Invitrogen) supplemented with 20% fetal bovine serum (FBS). Medium containing viral vectors was collected 48 and 72 hours after transfection. Viral vectors were filtered through a 0.45 nm syringe filter, aliquoted and stored at −80° C. Inducible TCF4 overexpression was achieved using a doxycycline-inducible vector system. Briefly, a TetR-T2A-NeoR insert was cloned inside a FUGW vector (Addgene Plasmid, Cat #14883). In a second plasmid, the expression of TCF4 was controlled by doxycycline through a TetO-regulated CMV promoter. Both vectors were transduced in MM011 or Ma-Mel-86c cells and selection was obtained with neomycin and puromycin respectively.

NY-ESO-1 A*02:01-SLLMWITQC (NYE 157-165; SEQ ID NO:1; also referred to as “NYE” hereinafter) expression was achieved using a pLVX-CMV-ZsGreen plasmid. Selection was obtained with puromycin. Inducible TCF4 downregulation was achieved using a doxycycline-inducible vector system. Briefly, the shRNA sequence targeting TCF4 was designed based on the sequence of the siRNA pool and cloned in a FH1 vector (Addgene Plasmid, Cat #164098).

The lentiviral vectors used to downregulate TCF4 in the in vivo mouse experiment (see 1.34 and Example 7) pLV[shRNA]-Bsd-U6>mTcf4 [shRNA #1] and its relative control pLV [shRNA]-Bsd-U6>Scramble_shRNA #1 were constructed and packaged by VectorBuilder. The vector ID are respectively VB230130-1137wap and VB010000-0007mbh, which can be used to retrieve detailed information about the vector on vectorbuilder.com. Selection was obtained with blasticidin.

### 1.20. Bulk RNA Sequencing

Approximately 2*10⁵ cells were plated in a 6-well plate. For knockdown experiments, these were transfected with the described siRNA pool 24 and 72 hours after plating and collected 24 hours after the second transfection. For inducible TCF4 experiments, cells were treated with 2 ng/ml doxycycline (Sigma-Aldrich, Cat #D9891) every 48 hours and collected 96 hours after plating. For ARV-771 experiments, cells were treated with 50 nM ARV-771 (Bioconnect, Cat #HY-100972) 24 hours after plating and collected 96 hours after plating. RNA was extracted using the RNA NucleoSpin extraction kit (Macherey&Nagel) according to the manufacturer's instructions.

The RNA integrity was monitored using Bioanalyzer analysis. 5 ng of RNA per sample was reverse-transcribed and amplified using a modified version of the SMARTseq2 protocol, previously described in Rambow et al. 2018 (Cell 174:843-855.e19). Prior to generating sequencing libraries using the NexteraXT kit (Illumina, Cat #FC-131-10), cDNA profiles were monitored using the Bioanalyzer. Sequencing was performed on a Illumina Nextseq500 platform. Differential gene expression analyses were executed using the DeSeq2 pipeline.

### 1.21. Geneset Enrichment Analysis

Geneset enrichment analysis was performed using GSEA 4.1.0. Briefly, approximately 3000 DEGs (si TCF4 vs si Ctrl) were ranked by log 2FC and the overlap with the following gene sets was estimated (MsigDB ID: M5930, M983 and M518).

### 1.22. Western Blotting

Harvested cell culture pellets were resuspended in protein lysis buffer (25 mM HEPES pH 7,5; 0,3 M NaCl; 1.5 mM MgCl2; 2 mM EDTA; 2 mM EGTA; 1 mM DTT; 1% Triton X-100; 10% glycerol; phosphatase/protease inhibitor cocktail), incubated on ice (10 min) and centrifuged at 14000 rcf for 15 minutes at 4° C. Equal amounts of protein, quantified using aLife Technologies Qubit 2.0 instrument were run on 4-12% Bis-Tris Plus Bolt gels (ThermoFisher Scientific) and transferred to a nitrocellulose membrane with an iBlot dryblot system (ThermoFisher Scientific). Membrane blocking (5% milk/TBS-0.2% Tween) was followed by incubation with the appropriate primary antibodies and HRP-conjugated secondary antibody. Signals were detected by enhanced chemiluminescence on Amersham hyperfilm.

Antibodies that were used are the following:
Rabbit polyclonal anti-MITF/Sigma-Aldrich Cat #HPA003259; RRID: AB_1079381
Rabbit monoclonal anti-TCF4/Abcam Cat #ab217668; RRID: AB_2714172
Rabbit monoclonal anti-GAPDH/Cell Signaling Cat #2118; RRID: AB_561053
Goat polyclonal anti-Rabbit IgG-HRP/Cell Signaling Cat #7074; RRID: AB_2099233

### 1.23. Colony Formation Assay

Cells were grown to near confluency on 12-well plates and treated on the following day with 2 ng/ml doxycycline (Sigma-Aldrich, cat #D9891) or vehicle. Forty eight hours after plating, cells were treated with the indicated drug combinations for four days. Cells were washed once with DPBS, stained with crystal violet (1% crystal violet w/v, 35% methanol v/v) for 15 minutes, washed with DPBS again and destained with tap water.

Three regions of interests were quantified using the ImageJ plugin ColonyArea (Guzman et al. 2014, PLOS One 9:1-9) to define the intensity percentage. Each sample was then normalized over the relative control well and statistical significance was assessed t-test (unpaired, two-tailed Student's t-test).

### 1.24. Co-Culture Experiments

The Nf-core hla typing analysis pipeline was used to determine the HLA profile from RNA sequencing data of the MM099 cell line (Ewels et al. 2020, Nat Biotechnol 38:276-278). HLA-matched peripheral blood mononuclear cells (PBMCs) and MM099 cells were grown in 5% CO2 at 37° C. for two days in RPMI 1640 medium supplemented with 10% FBS, 3 μg/mL anti-CD3 antibody (ThermoFisher Scientific, Cat #16-0038-85), 5 μg/mL anti-CD28 antibody (ThermoFisher Scientific, Cat #16-0289-85), 100 ng IL-2 (ThermoFisher Scientific, Cat #PHC0027).

TCF4 was silenced in MM099 cells as described above. Upon TCF4 knockdown, approximately 2000 MM099 cells per well were plated in a 96-well plate. Eight hours after plating, 10000 activated PBMCs per well were added, alongside aforementioned activating proteins and CellEvent™ Caspase-3/7 Green Detection Reagent (1:5000, ThermoFisher Scientific, Cat #C10423). Cells were imaged using the IncuCyte ZOOM System (Essen Bioscience) and automated apoptosis measurements were obtained based in images taken at two hour intervals, for the duration of the experiment. Three biological replicates were averaged and normalized over the last time point of the control and statistical significance was assessed (paired, two-tailed Student's t-test).

### 1.25. OmniATAC-Seq

Approximately 2*105 MM057 cells were plated in a 6-well plate and treated with ARV-771 100 nM after 24 hours. Forty eight hours after plating, cells were collected. Nuclei of 50,000 cells were isolated and an Omni-assay for transposase-accessible chromatin using sequencing (OmniATAC-seq) was performed as described previously (Corces et al. 2017, Nat Methods 14:959-962). After final amplification, samples were cleaned up with MinElute (QIAGEN) and libraries were prepared using the KAPA Library Quantification Kit (supplier). Samples were sequenced on an IlluminaNextSeq 500 High Output chip. Briefly, reads were mapped to human genome (GRCh37) using STAR (2.7.1a-foss-2018a). Resulting BAMfiles were cleaned for duplicates using Picard (2.21.8-Java-1.8.0) and indexed. Mitochondrial reads were removed using SAMtools (1.9-20190828-foss-2018a) and BigWig files were created (deepTools/3.3.1-foss-2018a-Python-3.7.4). ATAC-seq peaks were identified and visualized using MACS2 peak calling (2.1.2.1-foss-2018a-Python-2.7.16) with single-end BAMPE parameters. Finally, BED files were created from broadPeak files using BEDTools (2.28.0-foss-2018a).

### 1.26. RT-qPCR

MM057 cells were plated and treated with the specified dose of ARV-771, as described above 48 hours after plating, cells were collected, resuspended in RA1 lysis buffer using the RNA NucleoSpin extraction kit (Macherey&Nagel) and processed according to manufacturer's instructions. RNA was quantified using a ThermoScientific NanoDrop 1000 and 500 to 2000 ng was reverse transcribed with a High-Capacity cDNA Reverse Transcription Kit (Life Technologies). qPCRs were run using the SensiFAST probe No-ROX kit (Bioline, Cat #BIO-86005) on a Roche Life Science LightCycler 384. Data processing with Biogazelle Qbase+3.1 software relied on normalization with a minimum of two reference genes.

### 1.27. Proliferation Assay

Roughly 400 MM029 cells per well were plated in a 96-well plate and treated with ARV-771 300 nM and/or Dabrafenib 50 nM+Trametinib 10 nM after 24 hours. Cells were imaged using the IncuCyte ZOOM System (Essen Bioscience) and automated cell confluency measurements were made using images taken at 2 hour intervals, for the duration of the experiments. Five technical replicates were averaged and statistical significance was assessed (paired, two-tailed Student's t-test).

### 1.28. TCGA SKCM Data Analysis

The SKCM raw count matrix composed of 375 samples was downloaded from Firehose. Raw TCF4 counts were calculated per sample and samples were grouped based on their phenotype (Verfaillie et al. 2015, Nat Commun 6:6683). Furthermore, log 2 transformed read counts of were compared in metastatic versus primary melanoma lesions. Correlation analysis between MITF and TCF4 mRNA levels in TCGA_SKCM was performed using cbioportal (Gao et al. 2013, Sci Signal 6, μl1; Cerami et al. 2012, Cancer Discov 2:401-404).

### 1.29. Matrigel Invasion Assay and Quantification

The invasive capacity of melanoma cells was determined by Matrigel transwell invasion assays using 0.8 mm BD BioCoatMatrigel Invasion Chambers (Corning, Cat #354480), according to manufacturer's guidelines. Briefly, TCF4 expression was knocked down in MM099 cells as described above. Next, cells were starved overnight with FBS- and L-glutamine-deprived medium. Around 2*10⁵ cells were plated in each chamber (coated with 25 μg Matrigel) in FBS-deprived medium, while 10% FBS- and 2.5% L-glutamine-enriched medium was used in the wells placed in the lower chamber. Uncoated inserts were used as a control for proliferation. 24 hours after seeding, membranes were stained with crystal violet. Non-invading cells remaining on the upper surface of the chamber were removed by scrubbing with a cotton-tipped swab. Three to four randomly selected images were acquired per well and the surface cells were counted with ImageJ. The surface occupied by invading cells was calculated relative to the total surface of the membrane. Experiments included biological triplicates and technical duplicates.

### 1.30. CCLE Data Analysis

TCF4 read counts were plotted for skin melanoma cell lines from the CCLE cohort (DepMap, Broad (2022): DepMap 22Q2 Public. figshare. Dataset. https://doi.org/10.6084/m9.figshare.19700056.v2) and correlated (Pearson correlation coefficient) with IC50s (μM) of BRAF (PLX4720) and MEK (AZD6244) inhibitors.

### 1.31. Tumor Infiltrating Lymphocyte (TIL) Activation Assay

Activation of TILs by autologous tumour cells was analysed using the previously described melanoma model Ma-Mel-86 (Schrörs et al. 2017, Oncotarget 8:28312-28327; Pieper et al. 2018, Oncoimmunology 7: e1450127). Ma-Mel-86 tumor cells transfected with a doxycycline-inducible TCF4 expression vector were pre-treated with or without doxycycline (2 μg/ml) for 5 days. This was followed by co-culturing with autologous tumor-reactive CD8+TILs at a 1:1 ratio in the presence of 10 μg/mL Brefeldin A (Sigma-Aldrich). After 4 hours, cells were fixed/permeabilized using the Fixation/Permeabilization Concentrate and Diluent kit (eBioscience) and stained with antibody cocktail containing anti-human CD3-Brilliant Violet 421 (1 μL/test), CD8-APC-Cy7 (1 μL/test), CD4-APC (1 μL/test), and IFNγ-PE or TNFα-PE (5 μL/test) antibodies (Biolegend). Stained cells were analysed in a Gallios flow cytometer (Beckman Coulter). Data analysis was performed using the Kaluza software (Beckman Coulter).

1.32. Cloning of T Cell Receptor (TCR) Sequences into Retroviral Vectors

NYE-TCR expressing Jurkat T-cells were kindly provided by Dr. Martin Pule and were generated as described (Philip et al. 2014, Blood 124:1277-1287; Thomas et al. 2019, Nat Commun 10:4451). Expression of CD8 and of TCRs was knocked out in Jurkat T cells, followed by introduction of the NYE-TCR. Sequences of the variable chains of the used NYE-TCR are indicated in Table A.

TABLE A
DNA and amino acid sequences of the
NYE-TCR Vα and Vβ chains.

| TCR | DNA sequence | Protein sequence |
|---|---|---|
| NY-ESO- | TCTGTGCCTGAGGGCGAAAA | RSTLYIAASQPGDSATYLCA |
| 1_A*02_1G4_ | CCTGGTGCTGAACAAGCAAG | VRPLKQEVTQIPAALSVPEG |
| TCR-Vα | AAGTGACACAGATCCCTGCC | ENLVLNCSFTDSAIYNLQWF |
| | GCTCTGTGCAGCTTCACCGA | RQDPGKGLTSLLLIQSSQRE |
| | CAGCGCCATCTACAACCTGC | QTSGRLNASLDKSSGYGGSY |
| | AGTGGTTCAGACAGGACCCC | IPTFGRGTSLIVHPY |
| | GGCAAGGGACTGACAAGCCT | (SEQ ID NO: 3) |
| | GCTGCTGATTCAGAGCAGCC | |
| | AGAGAGAGCAGACCAGCGGC | |
| | AGACTGAATGCCAGCCTGGA | |
| | TAAGTCCTCCGGCAGAAGCA | |
| | CCCTGTATATCGCCGCTTCT | |
| | CAGCCTGGCGATAGCGCCAC | |
| | ATATCTGTGTGCCGTGCGAC | |
| | CTCTGTACGGCGGCAGCTAC | |
| | ATCCCTACATTTGGCAGAGG | |
| | CACCAGCCTGATCGTGCACC | |
| | CCTAC | |
| | (SEQ ID NO: 2) | |
| NY-ESO- | AAAACCGGCCAGAGCATGAC | ASSYVGNTGELFFGEGSRLT |
| 1_A*02_1G4_ | CCTGCAGTGCGCCGGCGTGA | VLGVTQTPKFQVLKTGQSMT |
| TCR-Vβ | CCCAGACACCTAAGTTCCAG | LQCAQDMNHEYMSWYRQDPG |
| | GTGCTGCAGGATATGAACCA | MGLRLIHYSVGAGITDQGEV |
| | CGAGTACATGAGCTGGTACA | PNGYNVSRSTTEDFPLRLLS |
| | GACAGGACCCTGGCATGGGC | AAPSQTSVYFC |
| | CTGAGACTGATCCACTATTC | (SEQ ID NO: 5) |
| | TGTCGGAGCCGGCATCACCG | |
| | ACCAGGGCGAAGTTCCTAAT | |
| | GGCTACAACGTGTCCAGAAG | |
| | CACCACCGAGGACTTCCCAC | |
| | TGAGACTGCTGTCTGCCGCT | |
| | CCTAGCCAGACCAGCGTGTA | |
| | CTTTTGTGCCAGCAGCTACG | |
| | TGGGCAACACCGGCGAGCTG | |
| | TTTTTTGGCGAGGGCAGCAG | |
| | ACTGACCGTGCTC | |
| | (SEQ ID NO: 4) | |

### 1.33. NYE-Expressing A375 (NYE-A375) Human Melanoma Cell and NYE-TCR-Expressing (NYE-TCR) Jurkat Cell Co-Culture Experiments

For co-culture experiments, NYE-A375 human melanoma cells were collected 48 hours after transfection with siCTRL or siTCF4 in the presence of IFN-γ. 5×10⁵ NYE-A375 cells were plated with 1×10⁶ NYE-TCR Jurkat cells in 200 μL of RPMI/DMEM (1:1) supplemented with 10% FBS and 1% Penicillin-Streptomycin per well of a 96-well plate. NYE-TCR Jurkat single-cultured cells were used as negative control. PMA (phorbol 12-myristate 13-acetate; 50 ng/ml, Sigma-Aldrich) and ionomycin (1 μg/mL, Sigma-Aldrich) were added to NYE-TCR Jurkat single-cultured cells as positive control.

Twenty-four hours after co-culture, NYE-TCR Jurkat cells were collected, washed twice in FACS buffer (PBS supplemented with 1% BSA (Sigma-Aldrich) and 2.5 mM EDTA (Sigma-Aldrich)). Cells were then stained with BB700 Mouse Anti-Human CD3 (Clone SK7, BD Biosciences, 1:200), BV421 Mouse Anti-Human CD8 (Clone RPA-T8, BD Biosciences, 1:200), BV650 Mouse Anti-Human CD69 (Clone FN50, BD Biosciences, 1:100), and eBioscience™ Fixable Viability Dye eFluor™ 780 (ThermoFisher Scientific, 1:1000) and incubated at 4° C. in the dark for 30 minutes. Cells were then washed twice in FACS buffer and resuspended in FACS buffer for flow cytometry. All experiments were run on a BD LSRFortessa™ X-Cell Analyzer and data was analysed with FlowJo v10.8.1.

### 1.34 In Vivo Knockdown of TCF4 Expression in Mice

All of the mouse colonies were maintained in a certified animal facility in accordance with European guidelines. Specifically, animals were housed in a controlled environment under 14 h-10 h light-dark cycles, standard diet and water ad libitum. All of the experiments strictly complied with the protocols approved by the University of Leuven Animal Care and Use ethical committee.

2*10\hat{ }5 B16F10 melanoma cells expressing an shRNA against TCF4 or an shRNA control were subcutaneously injected into 8-10 weeks old C57BL/6 male mice. 5 days after injection, treatment with anti-PD-1 (RMP1-14 or IgG2a isotype CTRL, BioXCell; 100 μg in 100 μl) was initiated. Tumour volume was monitored using callipers and the volume was calculated using the following formula: V=1/6*π*width*length*(width×length/2).

### Example 2. Portraying the Treatment-Naive Human Melanoma Transcriptomic Landscape

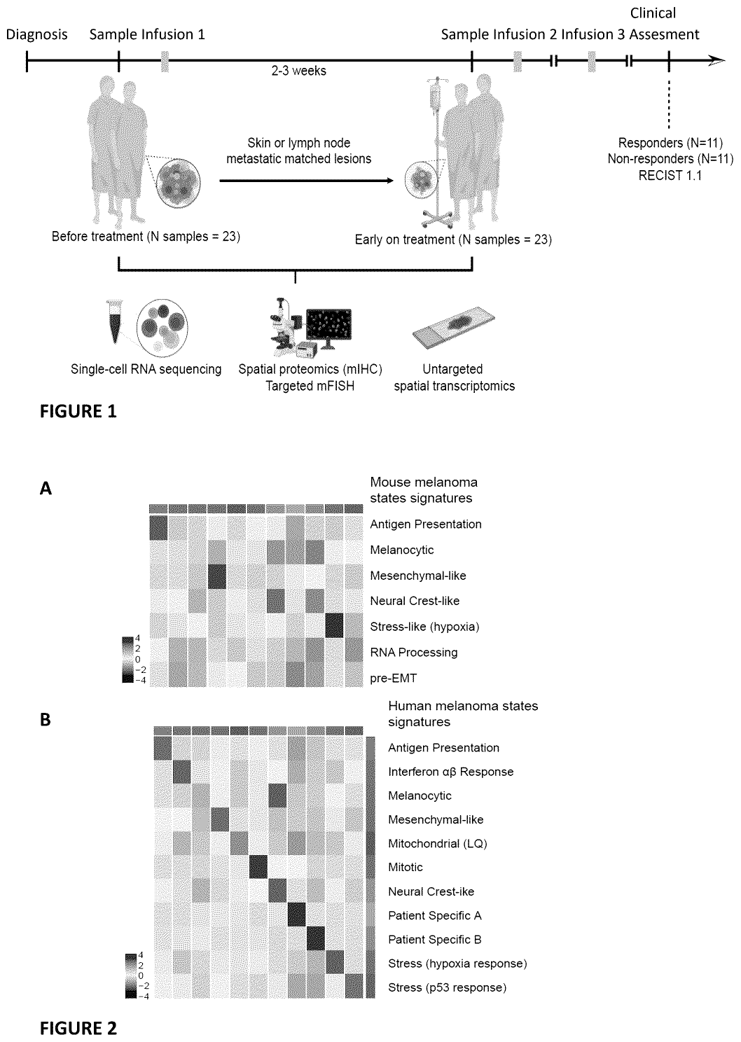

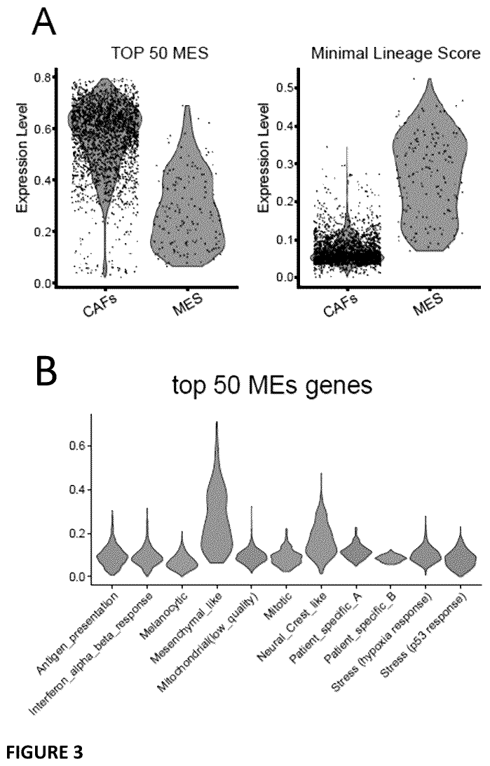

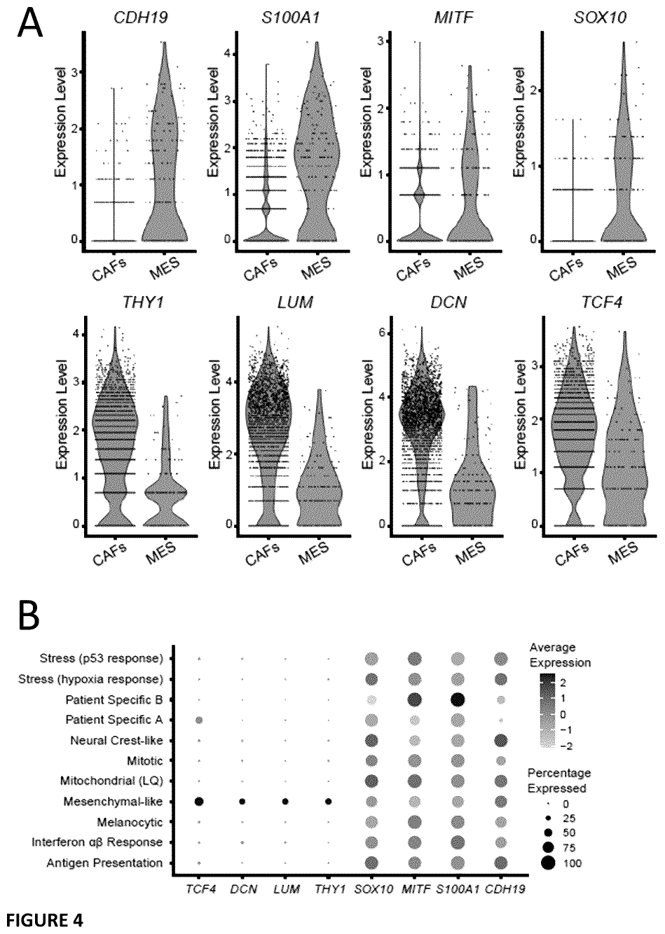

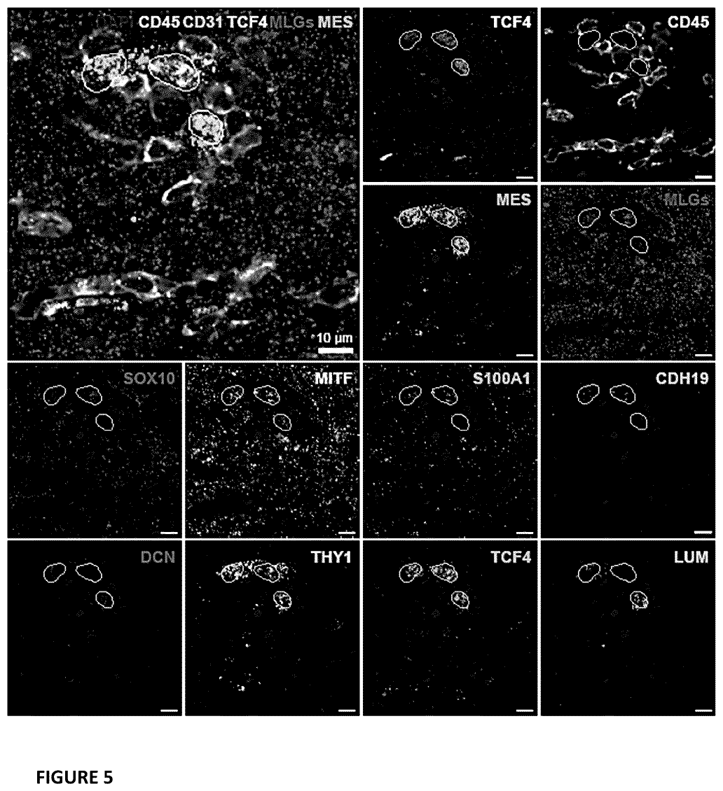

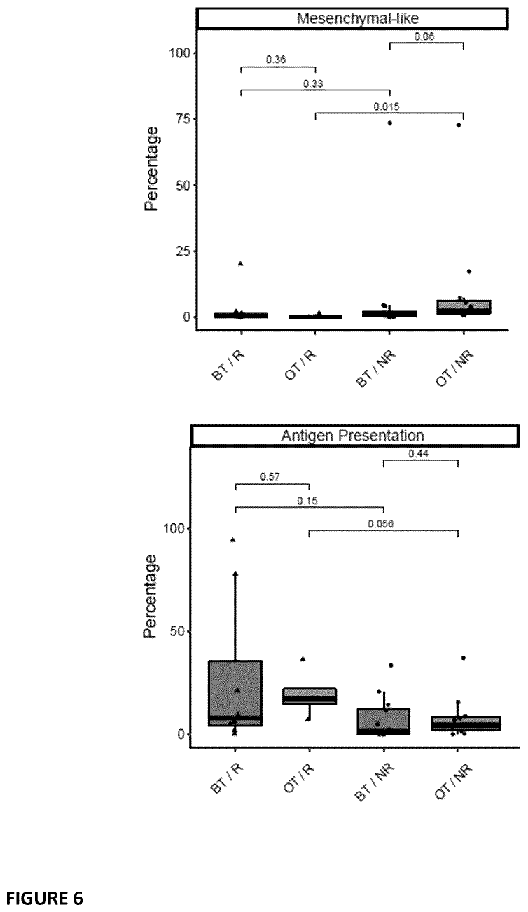

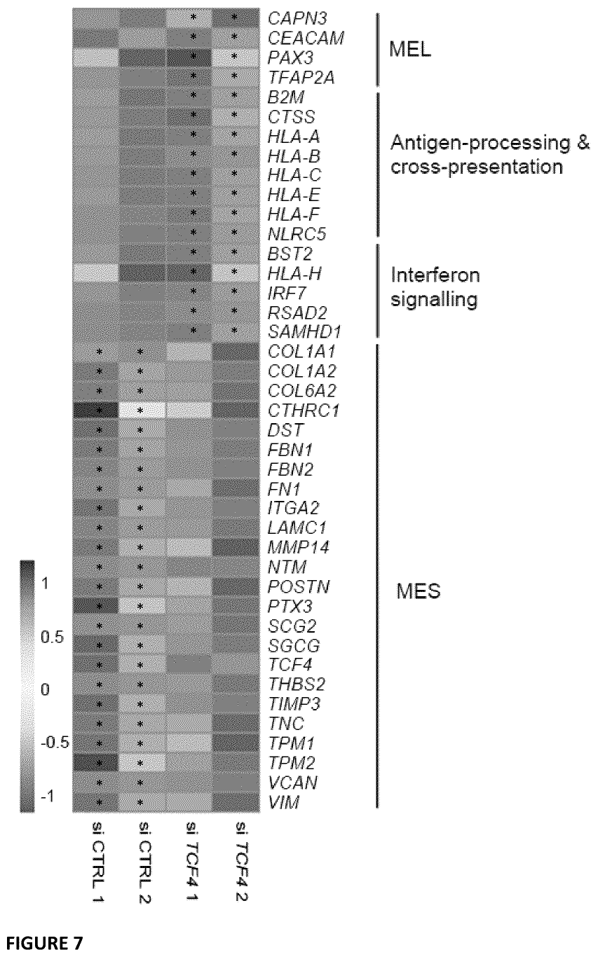

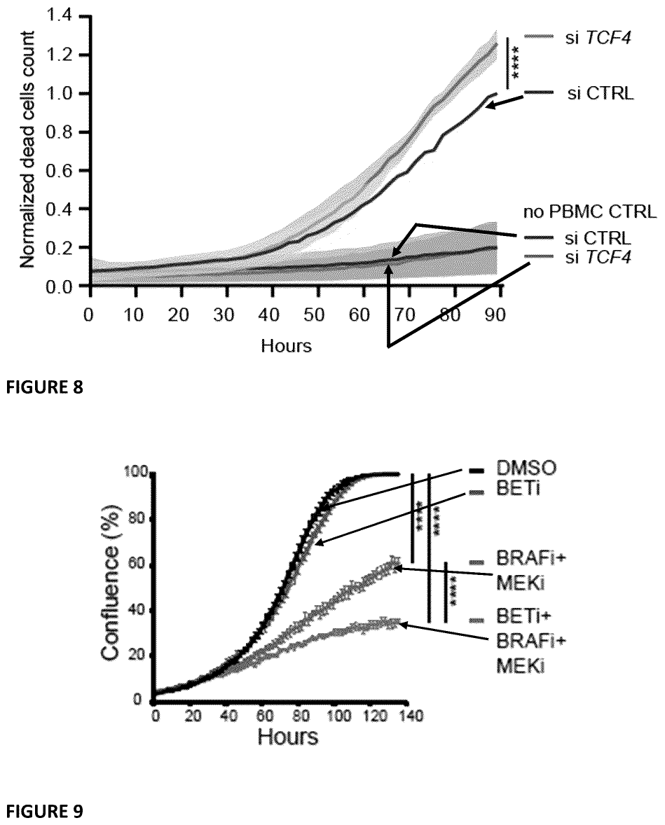

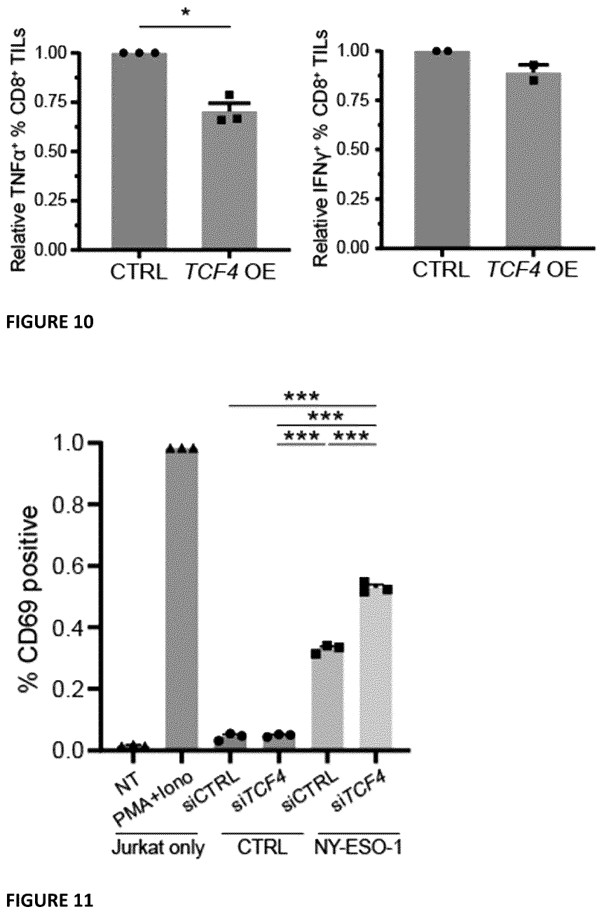

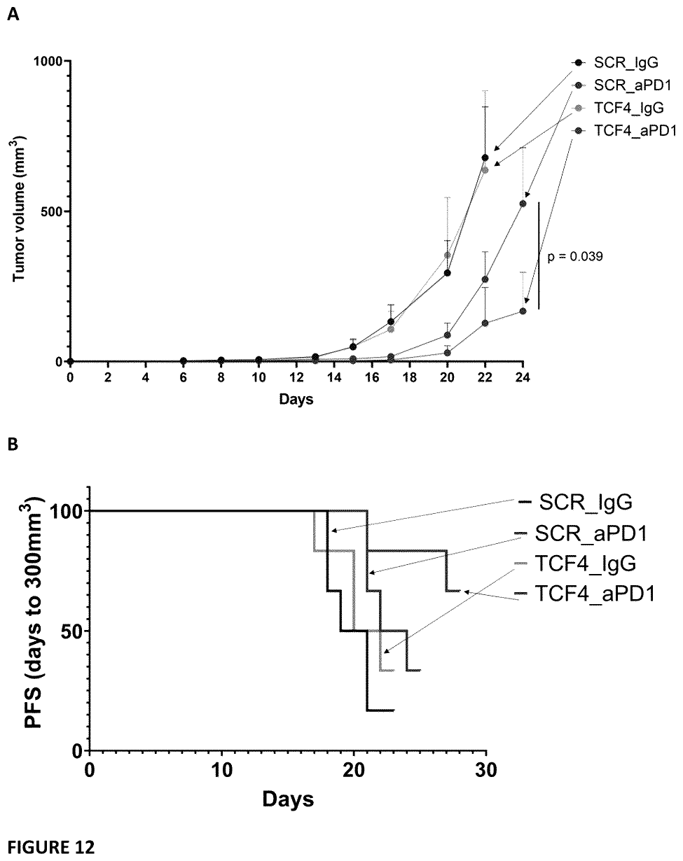

To dissect the cellular composition of the human melanoma ecosystem and study how it evolves under ICB, we initiated a prospective longitudinal study including treatment naïve stage III/IV (AJCC 8^{th} edition) melanoma patients receiving anti-PD-1 based therapy (anti-PD-1 monotherapy: n=17; ipilimumab+nivolumab combi therapy (n=6) SPECIAL; UZ/KU Leuven #S62275). Cutaneous, subcutaneous or lymph node metastases were biopsied before initiation of therapy (BT: before treatment). Subsequently, a second tumor biopsy was collected right before the administration of the second ICB treatment cycle (OT: on-treatment). We obtained patient- and lesion-matched biopsied across both time points for 20 patients. Part of the obtained material was preserved for routine pathological assessment, multiplex immunohistochemistry (mIHC), multiplex RNA fluorescence in situ hybridization (mFISH) and untargeted spatial transcriptomics. The remaining tissue was processed for single-cell transcriptome profiling (FIG. 1). Demographic, clinical, histopathological and genetic information was collected at baseline. Patients with unresectable disease were stratified as responders (complete remission, partial remission) and non-responders (stable disease, progressive disease) based on RECISTv1.1. best overall response, whereas patients treated with curative intent were stratified according to pathological response assessment at tumor resection (Eisenhauer et al. 2009, Eur J Cancer 45:228-247). Each of the four molecular subtypes (e.g. BRAF-, NRAS-, NF1-mutant and triple wild-type melanoma) were represented in this cohort at the expected frequency.

In total, >59K single cells passed our quality control requirements. To dissect the cellular composition of the melanoma tumor immune microenvironment (TIME), we first measured the activity of previously described stromal, immune and malignant/melanoma gene sets (Tirosh et al. 2016, Science 352:189-196) and assigned each cell from the unsupervised clusters to one of these three compartments. Importantly, each of these compartments could be detected in all lesions, irrespective of their metastatic site of origin.

We further refined our malignant cell annotation pipeline to filter out cells that do not exhibit large-scale genomic rearrangements, and do not harbour a high score for the malignant signature described by Jerby-Arnon et al. 2018 (Cell 175:984-997.e24) or for the “Melanoma Signature”, which we generated to discriminate between dedifferentiated melanoma cells and cancer-associated fibroblasts (see methods and below). We also excluded cells expressing the immune cell marker gene PTPRC (CD45).

As previously reported for melanoma (Jerby-Arnon et al. 2018, Cell 175:984-997.e24; Tirosh et al. 2016, Science 352:189-196; Sade-Feldman et al. 2018, Cell 175:998-1013.e20) and other human cancers (Wu et al. 2021, Nat Genet 53:1334-1347; Neftel et al. 2019, Cell 178:835-849.e21), dimension reduction visualisation showed a clear separation of malignant cells per patient, thereby impeding identification of shared transcriptomic states. This was partly overcome by regressing for patient ID and integrating the data using Harmony (Korsunsky et al. 2019, Nat Methods 16:1289-1296). Silhouette (Rousseeuw 1987, J Comput Appl Math 20:53-65) scores were measured to identify the optimal clustering resolution, which we initially set to 12 Seurat malignant clusters. Differential gene expression (DEG) analysis resulted in characteristic gene lists for each cluster. This analysis prompted us to merge cluster 0 and cluster 2 as they exhibited a similar enrichment for ribosomal genes, thus yielding 11 distinct malignant clusters.

For an in-depth characterisation of the treatment-naïve melanoma ecosystem, we perform another DEG analysis focusing on the untreated samples and interpreted the differentially expressed genes lists using EnrichR (Xie et al. 2021, Curr Protoc 1: e90-e90) across multiple databases. Gene regulatory modules were also defined for each cluster using SCENIC (Aibar et al. 2017, Nat Methods 14:1083-1086). For the functional annotation of malignant clusters we relied both on the interpretation of DEG lists, with various gene set enrichment tools, and prior biological knowledge acquired through analysis of a scRNA-seq dataset from Tyr::NRasQ61K/°; Ink4a−/−mouse tumors (Karras et al. 2022, Nature 610:190-198). Unsupervised clustering of these mouse lesions identified 7 distinct melanoma cell states, which we named Melanocytic, Mesenchymal-like, Neural-Crest-like, Stress (hypoxia response), RNA-processing, Stem-like (pre-EMT) and Antigen Presentation cell states. 6 of these 7 murine melanoma states overlapped with cellular states identified in the human lesions (FIG. 2A). These included the Melanocytic (MEL), Mesenchymal-like (MES), Antigen Presentation, Neural Crest-like, Stress (hypoxia response) cell states. The murine RNA processing state largely overlapped with the human Stress (p53 response) state. Among the previously described marker genes NGFR was identified in the Neural-Crest-like state, VEGFA was highly expressed in Stress (hypoxia response) cells, and PDGFR in MES cells. HLA class I and II and other genes involved in antigen processing and presentation, such as TAP1, B2M and NLRC5, were identified as discriminative markers of the Antigen Presentation cell population. The cross-species comparison also highlighted five human-specific cell states: an Interferon Alpha/Beta Response state expressing interferon type I responsive genes (i.e. IFI6, IFI27, IRF7), but not genes involved in antigen processing and presentation pathways. A Mitotic state, which expressed high levels of MKI67 and TOP2A, was also identified, as well as a Mitochondrial state, which exhibited high mitochondrial gene expression and showed no consistent pathway enrichment. We annotated this latter cell state as a “low quality” malignant cell cluster. Mitotic and Mitochondrial (low quality) cell clusters are both routinely identified in human tumor biopsy samples (Qian et al. 2020, Cell Res 30:745-762; Kinker et al. 2020, Nat Genet 52:1208-1218). Finally, two patient-specific clusters (Patient-specific A and Patient-specific B), which did not exhibit any specific recognizable functional features, also emerged at this level of resolution. Since these clusters were only detected in individual patients we postulate that they may be driven by specific genetic alterations. Note that while the murine pre-EMT stem-like state did not emerge as an independent cluster, supervised analysis highlighted human melanoma cells from different patients residing in this state (Karras et al. 2022, Nature 610:190-198).

Using the gene signature of each state we calculated signature scores per individual and visualised the score per state (FIG. 2B). While the cellular heterogeneity of melanoma cells broadly aligned with the 11 cell states, a substantial fraction of cells was not exclusively constraint to these states, indicating that melanoma cells can manifest multiple and/or overlapping phenotypes.

The MITF rheostat model predicts that melanoma cell state identity is, by and large, regulated by the activity of the MITF transcription factor (TF) (Rambow et al. 2019, Genes Dev 33:1295-1318). The proliferative/melanocytic and de-differentiated invasive/mesenchymal-like states exhibit high and low MITF activity, respectively. Measuring the activity of these gene expression programs (Hoek et al. 2008, Cancer Res 68:650-656; Verfaillie et al. 2015, Nat Commun 6:6683) across all malignant cells confirmed that cells with varying MITF activity co-exist in drug-naïve human metastatic melanoma lesions (data not shown). As expected, the Neural-Crest and MES states were the most de-differentiated states. We also measured the activity of a series of previously published melanoma transcriptional cell states (Tsoi et al. 2018, Cancer Cell 33:890-904.e5; Rambow et al. 2018, Cell 174:843-855.e19; Wouters et al. 2020, Nat Cell Biol 22:986-998; Baron et al. 2020, Cell Syst 11:536-546.e7) identified in various cellular and/or in vivo model systems and confirmed their identity and/or presence in clinical samples (Jerby-Arnon et al. 2018, Cell 175:984-997.e24).

We next grouped all malignant cells in distinct CNV genomic clusters. An alluvial plot was used to connect the genomic and transcriptomic clusters for each cell. Except for cells from the patient specific clusters A and B, all transcriptional clusters were fed with cells from different genomic clusters. Moreover, we did not observe any association between the abundance of a particular melanoma cell state and a specific oncogenic driver mutation. Together these data identified several shared and evolutionarily conserved melanoma transcriptional metaprograms, which do not appear to be driven by genetic intratumor heterogeneity, but instead are likely to be specified by cues emanating from the tumor immune microenvironment.

A list of mesenchymal-like MES cell markers is given in Table 1. A list of markers for the antigen-presenting cell state is given in Table 2.

TABLE 1
Mesenchymal-like (MES) cell markers.

PDGFRB, NRP1, COL5A1, COL1A1, COL3A1, LUM, THY1, TCF4, ID3, PMEPA1, MRC2, DCN, COL1A2,
COL4A1, MXRA8, TMSB4X, COL6A2, COL6A3, COL5A2, COL4A2, COL6A1, AQP1, FBLN1, IGFBP7,
COL12A1, BGN, PCOLCE, VCAN, ANTXR2, PBX1, HTRA1, C1R, SELENOM, FBN1, SPARC, MARCKS, FN1,
COL15A1, PALLD, CALD1, ITGA1, POSTN, CFH, PTN, ITGB1, H3F3B, TPM2, TIMP1, TMEM47, PRRX1,
PTMA, C1S, COL18A1, TUBA1A, TPM1, IGFBP2, AHNAK, CCDC80, TMSB10, MMP2, IGFBP4, CD9,
PGRMC1, NR2F2, TPM4, CNN3, S100A4, LAMB1, EMP2, HMGB1, GSN, CTHRC1, TGFBR2,
TNFRSF12A, FGFR1, S100A6, IFITM2, LGALS1, VCL, LAMC1, TGFBI, MGP, PTMS, ANXA2, ANXA1,
LAPTM4A, GNG11, ITM2B, TIMP3, LHFPL6, H3F3A, FERMT2, DEK, FSTL1, ARGLU1, RAP1B, UTRN,
MYL9, CAVIN1, NDRG2, ZFP36L2, PPIB, UBC, PNRC1, FAM133B, QKI, PRNP, NFIB, AKAP13, RUNX1,
RHOB, MATN2, PRSS23, MAP1B, COL9A3, CYBRD1, PHACTR2, JAG1, PLPP1, TAGLN2, LIMA1, SPRY1,
STARD13, SH3PXD2A, MPZ, ENG, SH3BP5, RGS16, BZW1, CTGF, MDK, UACA, SPTBN1, LIMS1,
PMP22, TNC, LRP1, SERPING1, LTBP4, ATP2B1, SOX4, TXNIP, ITM2C, HSPG2, RNF24, SSFA2, CEBPD,
FOXP1, HDGF, S100A10, HIF1A, BAZ1B, CST3, KCTD12, NCOA7, UBL5, CSRP1, TOP1, GNAS, FXYD1

### Mesenchymal-Like (MES) Cell Markers Common to Human and Murine Tumors are Listed in Table 3.

TABLE 3
Mesenchymal-like (MES) cell markers common to human and murine tumors.

PDGFRB, NRP1, COL5A1, COL1A1, COL3A1, LUM, THY1, ID3, MRC2, DCN, COL1A2, COL4A1,
COL6A2, COL6A3, COL5A2, COL4A2, COL6A1, IGFBP7, BGN, PCOLCE, VCAN, FBN1, SPARC,
COL15A1, ITGA1, CFH, PRRX1, TCF4

### Example 3. Spatially Mapping of Melanoma Cell State Diversity

To gain insights into the spatial organization of the various melanoma cell states in drug-naïve lesions, we performed untargeted spatially resolved transcriptomics on selected samples (n=6; S1 to S6) from our patient cohort, using the 10× genomics Visium platform. Each section was annotated by a pathologist based on the morphology of the associated hematoxylin and eosin (H&E) staining. Regions were labelled as either malignant, stromal or immune.

On the Visium platform, multiple (often different) cell types contribute to the transcription profile of each spot (˜0-200 cells/spot). Therefore, to properly capture the nuances of each patient's molecular profile, and to not risk quenching weak signals, each slide/patient was analysed separately (Andersson et al. 2021, Nat Commun 12:6012). To spatially resolve the malignant cell states, the spatial transcriptomics data was integrated with the scRNA-seq data using Seurat-v3 anchor-based integration (Stuart et al. 2019, Nat Rev Genet 20:257-272) and CellTrek (Wei et al. 2022, Nat Biotechnol 40:1190-1199) deconvolution methods.

The Seurat anchor-based integration confirmed that spots in cancer regions were highly enriched for the malignant signature and spots falling outside of the malignant areas were enriched in stromal and/or immune cells (data not shown). These findings were considered as affirmative of our mapping's validity. Zooming in into the cancer regions revealed that, by and large, melanoma transcriptional metaprograms are not randomly distributed but tend to co-occur in spatially restricted clusters. The differential spatial distribution of the malignant cell states can be further highlighted through co-localisation analyses. For instance, as opposed to Mesenchymal-like cells, cells harbouring the Neural Crest-like state preferentially co-localised with Antigen presentation cells.

Using the scRNA-seq data, we further established a strong positive correlation between the percentage of cells harbouring the Antigen Presentation state and activated CD8 T cells (R=0.64, P=0.0023, FIG. 2C). Quantitative inference and analysis of intercellular communication networks predicted a functional interaction between these two cell types through engagement of the MHC class I and II signalling pathways. Consistently, the Antigen Presentation cell state was enriched in lesions with an immune inflamed, also described as brisk, phenotype. To further establish a spatial relationship between these two cell types, we performed mIHC using multiple iterative labelling by antibody neodeposition (Bolognesi et al. 2017, J Histochem Cytochem 65:431-444) (MILAN) on treatment naïve melanoma samples (n=20). Neighbourhood analysis confirmed enrichment of melanoma cells positive for the MHC class II marker HLA-DR in the proximity of CD8 T cells (Tcy). In contrast, HLA-DR-negative melanoma cells and T cells occur in mutually exclusive regions.

Together, these findings indicate that the transcriptomic heterogeneity of melanoma is spatially organized within the tumor architecture and is, at least partly, driven by heterotypic cellular interactions with the tumor immune microenvironment (TIME). For instance, by integrating signalling predictions with cellular proximity, these data indeed raise the possibility that the melanoma antigen presentation cell population emerge by direct interaction with immune cells (i.e. T-cells).

### Example 4. Unambiguous Detection of Melanoma MES Cells

Similar to epithelial cancer cells that have undergone Epithelial-to-Mesenchymal Transition (EMT), melanoma cells that acquired a mesenchymal-like/de-differentiated phenotype closely resemble normal mesenchymal cells in the TIME, CAFs in particular (Rambow et al. 2019, Genes Dev 33:1295-1318; Pastushenko et al. 2019, Trends Cell Biol 29:212-226). Findings concerning EMT through the analysis of bulk-level expression data from human tumors have therefore been confounded by the presence of CAFs (Tyler & Tirosh 2021, Nat Commun 12:2592). Moreover, identification of coherent and specific marker gene sets that distinguish CAFs and malignant cells that underwent EMT has been a major challenge in the field. Recently, an approach for decoupling the mesenchymal expression profiles of cancer cells and CAFs leveraging scRNA-seq datasets was developed and applied to various epithelial cancers (Tyler & Tirosh 2021, Nat Commun 12:2592). Unexpectedly, there was no clear evidence for a full EMT malignant state, indicating that this state either does not exist, or is extremely rare and/or transient. Instead, cancer cell-specific partial EMT (pEMT) programs that are distinct from CAF signatures were defined. Even more surprisingly, pEMT was not associated to any specific clinical features across cancers, thereby indicating that the clinical relevance of pEMT expression programs may be highly context-specific. Our single cell analyses did, however, identify melanoma cells expressing a full MES program in both human and mouse (Karras et al. 2022, Nature 610:190-198) datasets. In our human dataset, the 50 most abundantly expressed genes in MES cells were remarkably almost all highly expressed in CAFs (FIG. 3A, 3B). In order to define a melanoma-specific mesenchymal-like gene expression signature, we established a list of the most differentially expressed genes between melanoma MES cells and CAFs (Table 4; the top 50 most differentially expressed genes listed in Table 5).

TABLE 4
List of most differentially expressed genes between melanoma MES cells and CAFs.

ERBB3, CDH19, PTPRZ1, PLP1, SOX10, EDNRB, S100B, ST3GAL6, ADGRG1, MCAM, LGI4, SOX6,
TFAP2A, ITGA6, SORBS2, S100A1, DUSP4, PRAME, GPM6B, SNCA, ATP1B1, AP1S2, TUBB2B, MIA,
CHD7, PAX3, LYST, SYNM, CRYAB, SEC11C, CADM1, COL9A3, SERPINE2, TYR, MICAL3, DCT, MEF2C,
FMN1, SSFA2, ATP1A1, HSP90AB1, SORT1, STMN1, SYT11, PLOD3, HSP90AA1, MLANA, PHACTR1,
PLAT, PDGFA, ENDOD1, MYO10, GULP1, GPR155, MT-ND4L, SDCBP, ETV5, FIGN, SYNGR1, RHOB,
SLC23A2, NES, CSRP1, PMEL, DUSP6, HMG20B, MARCKSL1, SDC3, TOMM20, ASAH1, CNP, PUF60,
HACD3, SDHC, MATN2, FAM210B, ISYNA1, HSPE1, CSPG4, NDUFA4, CD320, SLC3A2, ATP6V0B,
TSC22D4, HSPD1, NDUFB9, FBXO7, SLC25A4, MITF, MT-CO2, UHMK1, SOX4, STARD13, POLR2F,
SARAF, HSPA12A, COMT, GPNMB, SYPL1, PHLDA1, PPIA, C1orf43, MXI1, KMT2E, MAGED2,
GPR137B, PCDH7, RPS7, APP, ATP6V1G1

TABLE 5
List of top 50 most differentially expressed genes between melanoma MES cells and CAFs.

ERBB3, CDH19, PTPRZ1, PLP1, SOX10, EDNRB, S100B, ST3GAL6, ADGRG1, MCAM, LGI4, SOX6,
TFAP2A, ITGA6, SORBS2, S100A1, DUSP4, PRAME, GPM6B, SNCA, ATP1B1, AP1S2, TUBB2B, MIA,
CHD7, PAX3, LYST, SYNM, CRYAB, SEC11C, CADM1, COL9A3, SERPINE2, TYR, MICAL3, DCT, MEF2C,
FMN1, SSFA2, ATP1A1, HSP90AB1, SORT1, STMN1, SYT11, PLOD3, HSP90AA1, MLANA, PHACTR1,
PLAT, PDGFA

Several of these genes including CDH19 and S100A1, which we termed Minimal Lineage Genes (MLGs), were identified in both mouse and human MES signatures and were indeed highly and selectively expressed in MES cells (FIG. 4A). Importantly, expression of these genes was higher than MITF and SOX10, two melanoma markers known to be expressed at very low to undetectable levels in the de-differentiated MES cells. In contrast, whereas stromal genes like THY1, LUM and DCN were expressed at higher levels in CAFs than in MES cells, several markers including the basic helix-loop-helix (bHLH) transcription factor TCF4 (also known as ITF2 or E2-2) was instead expressed at comparable levels in both cell types (FIG. 4A). Note that, consistent with previous findings (Cisse et al. 2008, Cell 135:37-48), TCF4 expression was also detected in endothelial (ECs) and plasmacytoid dendritic cells (pDCs; data not shown). Measuring expression of these genes in all melanoma states revealed that whereas CDH19 and S100A1 were expressed in all subtypes (including MES cells), TCF4 and other stromal genes were selectively expressed in melanoma MES cells (FIG. 4B). We concluded that the MLGs provides the field with a unique tool to unambiguously discriminate between de-differentiated/mesenchymal-like melanoma cells and CAFs in both mouse and human single-cell datasets.

We next sought to devise a method for the detection and mapping of Mesenchymal-like cells in situ. Because expression of the MLGs is relatively low in MES cells, we opted to combine a highly sensitive detection mFISH-RNAscope method with an oligonucleotide barcoded antibody-based mIHC protocol (AKOYA/CODEX). Guided by our scRNA-seq data, we designed a mFISH panel selecting the most discriminatory MLGs (S100A1 and CDH19) and MES (THY1, DCN, LUM) markers to complement a broad mIHC panel targeting selected melanoma, immune and stromal protein markers. We included the pan-mesenchymal marker TCF4, as well as MITF and SOX10, in both our mIHC and FISH panels.

We first performed mIHC followed by mFISH on a selected treatment naïve melanoma lesion. MES cells were identified by co-staining of MLGs (CDH19 and S100A1) and melanoma (MEL) markers (MITF and SOX10) with MES markers (DCN, THY1, LUM and TCF4) within the CD45-negative cell population. Instead, CAFs were positive for the MES markers and negative for MLGs. Other melanoma subpopulations were positive for the MEL and MLG markers and negative for the MES markers (FIG. 5). Note that pDCs were identifiable as CD45+CD31−MES−MLGs−, and ECs were CD45−CD31+MES+MLGs− (data not shown).

To further validate our method, we selected another melanoma sample (GC34) because it was identified as particularly rich in melanoma MES cells by scRNA-seq and it harboured the BRAF^{V600E} mutation. We stained adjacent sections with our combined mIHC and mFISH protocol, and with an antibody directed against the oncogenic form of BRAF^{V600E} (as substitute for the melanoma cell markers). As expected, this sample contained a very high proportion of cells identified as melanoma MES cells. These cells also stained for the BRAF^{V600E}-specific antibody, thus further confirming their melanoma origin. Together, these data provide a new method to unambiguously identify true melanoma MES cells in both scRNA-seq datasets and on tissue sections, and firmly establish the presence of these cells in human treatment-naïve melanoma lesions.

### Example 5. MES Cells are Enriched in Early On-Treatment Melanomas Refractory to ICB

Having established the cellular architecture of the drug-naïve melanoma ecosystem and the necessary tools for the unambiguous annotation of all malignant cell states, we next studied how one cycle of ICB therapy may remodel the melanoma transcriptional landscape. We did not observe any significant differences in the proportion of the various melanoma cell states between the BT and OT time points. Interestingly, however, two of the melanoma cell states, namely the Mesenchymal-like and Antigen Presentation states, were quantitatively impacted and associated with divergent clinical responses (FIG. 6). Whereas the Antigen Presentation cell state was enriched in OT samples from responders (R), the MES cells were significantly enriched in OT samples from non-responders (NR). The trend of increased abundance of Antigen Presentation cells in lesions from R compared to NR was already observed BT, but this difference was further enhanced at the OT time point (FIG. 6). In contrast, the enrichment of melanoma MES cells in the NR lesions was only observed OT (Wilcoxon-test p=0.015). Importantly, the presence of these two cell populations in the OT samples showed a high diagnostic ability for response prediction and, thereby, biomarker potential. In contrast, none of the other melanoma cell states showed any significant associations with response.

### Example 6. TCF4 Orchestrates Multiple Melanoma Transcriptional Metaprograms

To assess the contribution of TCF4 in the establishment/maintenance of the MES transcriptional metaprogram, we performed bulk RNA-seq in the melanoma MES cell line MM099, following silencing of TCF4 expression. Genes downregulated upon TCF4 knockdown were involved in cellular movement, EMT, integrin signalling and angiogenesis, thus establishing its role as a driver of the mesenchymal-like (MES) transcriptional program (FIG. 7). This was concomitant to an upregulation of a series of MITF target genes and genes from the melanocytic (MEL) transcriptional program. This observation was consistent with a previous report indicating that TCF4 can repress MITF in normal melanocytes (Furumura et al. 2001, J Biol Chem 276:28147-28154). However, whether TCF4 represses MITF in melanoma cells is unknown. To test this hypothesis, we overexpressed TCF4 in two different melanocytic melanoma cell lines (MM001 and MM011) and observed a downregulation of MITF mRNA and protein levels as well as MITF target genes. These data indicated that, in addition to its function as a master regulator of the MES transcriptional program, TCF4 also actively suppresses the MITF-driven melanocytic transcriptional program.

Importantly, silencing TCF4 in MM099 caused a dramatic decrease in the ability to invade in short-term in vitro migration assays.

Consistent with the melanoma MES state being intrinsically resistant to MAPK therapeutics (Rambow et al. 2019, Genes Dev 33:1295-1318), an inverse correlation between the sensitivity to BRAF- and MEK-inhibitors and TCF4 expression was observed in the Cancer Cell Line Encyclopedia melanoma cell line cohort. Critically, silencing TCF4 sensitized the human melanoma BRAF^{V600E}-mutant invasive line MM099 to these inhibitors. These data indicated that TCF4 contributes to the acquisition and/or maintenance of the mesenchymal-like phenotype and thereby to resistance to targeted therapy.

Remarkably, and not previously reported, many genes involved in immune response (antigen processing and presentation, activation of leukocytes, and interferon signalling) were upregulated upon TCF4 knockdown (FIG. 7). This included the transcription factor NLRC5, a master regulator of MHC class I and class I-related genes (Cho et al. 2021, Immunology 162:252-261). Consistently, there was a strong enrichment of the Antigen Presentation and Interferon (IFN) signalling gene signatures among the genes upregulated upon TCF4 silencing (FIG. 7). Together, these data indicated that TCF4 actively suppresses the melanoma MEL, Antigen Presentation and Interferon signalling gene expression programs. By doing so, TCF4 appears to directly promote immune cell evasion and/or resistance to immunotherapy.

### Example 7. Targeting TCF4 Expression Increases Apoptosis of Mesenchymal-Like (MES) Tumor Cells, Increases T-Cell Activation, and Potentiates Therapy with Immune Checkpoint Blockers

By suppressing the antigen processing and presentation machinery, TCF4 further protects de-differentiated melanoma cells from T-cell killing. Consistent with this model, TCF4 silencing increased apoptotic cell death activation in a melanoma MES cell culture exposed to HLA-matched peripheral blood mononuclear cells (PBMCs), which were pre-treated with a T cell activating cytokine cocktail (FIG. 8). These results, together with the increased levels of MES-cells early on in patients not responding to ICB therapy, are further supportive of combining TCF4 inhibition with immune checkpoint blocking therapy (switching on T cells for attacking tumor cells) in the treatment of ICB-refractive tumors; or of inhibiting TCF4 to improve response to ICB.

To further illustrate the contribution of the activated T cells in inducing apoptosis of MES tumor cells, the MES tumor cells are incubated with (i) the HLA-matched PBMC from which T-cells have been removed (no increase in apoptosis of MES cells) and with (ii) isolated activated T cells (increase in apoptosis of MES cells).

Furthermore, TCF4 overexpression in the patient-derived melanoma cell line Ma-Mel-86c led to reduced activation of autologous T cells, as measured by a decrease in TNFα- and IFNγ-positivity (FIG. 10). In an alternative setting, immortalized Jurkat CD8−/−TCR−/−T cells transduced to stably express exclusively the TCR recognising the NY-ESO-1 A02 (1G4) peptide were co-cultured with A375 melanoma cells engineered to express the NY-ESO-1 A02 (1G4) peptide (NYE-A375). Consistently, silencing of TCF4 in NYE-A375 cells led to a marked decrease in activation of the NYE-TCR Jurkat cells as measured by the % of CD69-positive Jurkat cells (FIG. 11). Notably, this assay relied on the recognition by the T-cells of specific antigens (NY-ESO-1), discriminating the role of TCF4 on the melanocytic antigens from the role on the antigen presentation machinery.

Thus, the increased apoptosis rate of melanoma cells in which TCF4 expression is silenced by PBMCs (see above) can be linked with the activity of the T cells in the PBMC preparation, in particular with the activity of the activated T cells in the PBMC preparation.

Furthermore, a MaMEL cell-line (MAMel86c line (melanocytic, TCF4 low, Mitf high); a non-MES cell-type) expressing the melanocytic antigen targeted by T-cells is effectively killed by T-cells targeting the antigen. Induced overexpression of TCF4 (doxycline-inducible) in the MaMEL cell-line reduces killing by T-cells targeting the antigen.

Finally, an in vivo murine model with B16F10 melanoma cells expressing an shRNA against TCF4 or an shRNA control, TCF4 knockdown combined with immune checkpoint blocking therapy (anti-PD1) lead to an increase in response to anti-PD1 therapy when compared to the control condition. Indeed, FIG. 12 demonstrates that silencing TCF4 in B16F10 melanoma cells injected in C57BL/6 mice led to an increase in response to anti-PD1 therapy when compared to the control condition (shRNA scramble).

Other in vivo murine models that can be assessed include use of the NRASQ61K/°; Ink4a−/− melanoma tumor (Karras et al. 2022, Nature 610:190-198), YUMM1.7 and YUMMER1.7 (Zhang et al. 2021, Nature 598, 682-687) and YUMMER.G. (Ramseier et al. 2019, Cancer Res 79, 13 Supplement, 4622).

### Example 8. Targeting TCF4 Expression Through BET-Inhibition

TCF4 was shown to drive B cell lymphoma and blastic plasmacytoid dendritic cell neoplasm (BPDCN) (Jain et al. 2019, Sci Transl Med 11: eaav5599; Ceribelli et al. 2016, Cancer Cell 30:764-778). In these studies, TCF4 expression was shown to be dependent on the bromodomain and extra terminal domain (BET) protein BRD4 through its recruitment to a specific TCF4 enhancer region. Inhibition of BRD4 using the BET-degrader ARV-771 was shown to decrease its expression. Interestingly, bulk Assay for Transposase-Accessible Chromatin using sequencing (ATAC-seq) revealed that the BRD4-bound enhancer region upstream the TCF4 promoter is largely accessible in melanoma MES cells such as MM099 and MM057, but not in melanoma MEL cells such as MM001 and MM011. Exposure of the melanoma line MM057 to the BET-degrader ARV-771 decreased chromatin accessibility upstream of the TCF4 locus, including of one of the sites previously identified as a BRD4-bound enhancer region. Consistently, this treatment led to a dose-dependent decrease in TCF4 expression. Notably, the overall transcriptional reprogramming effect observed upon BET-inhibition was far more drastic in mesenchymal-like than in melanocytic cell lines, indicating that the MES transcriptional program may be particularly dependent on this pathway. Moreover, exposure to ARV-771 recapitulated most of the transcriptional changes observed upon TCF4 silencing. Most genes from the MES signature were strongly downregulated, whereas genes from the MITF-dependent MEL signature were upregulated. Importantly, just like upon TCF4 knockdown, these transcriptional changes were accompanied by an increased sensitivity to BRAF- and MEK-inhibition (FIG. 9). Moreover, the antigen presentation program was also upregulated upon exposure to ARV-771. This observation raises the possibility that this compound may also be used to increase the immunogenicity of MES cells and thereby their sensitivity of ICB.

### Example 9. Discussion

In this study, we portrayed the cellular architecture of treatment-naïve skin and lymph node melanoma metastases, and thereby provide the field with a rich resource which may serve as the foundation for the creation of a comprehensive cell atlas of melanoma. The data can be visualised, analysed and downloaded from an interactive web server (https://marinelab.sites vib.be/en/data-access). We focused our attention on the malignant compartment and show that the melanoma transcriptional landscape is more complex than previously assumed. We describe a series of recurrent cell states that are evolutionarily conserved and show that the spatial distribution of these distinct melanoma subpopulations is not random, suggesting that the tumor immune microenvironment directly contributes to this geographically organised transcriptomic heterogeneity. This is, for instance, illustrated by the proximity we describe between the Antigen Presentation cell population and T cells, which suggests that this particular transcriptional program may be acquired through an intercellular communication pathway established by T cells. In concordance with this hypothesis, we previously observed that engraftment of a homogeneous mouse melanocytic cell line into immune competent, but not immunodeficient, mice resulted in the formation melanoma lesions harbouring a complex and heterogenous transcriptomic landscape that included the Antigen Presentation cell state (Karras et al. 2022, Nature 610:190-198).

The functional contribution of these newly defined cell states to tumor growth and/or metastatic spreading can now be explored. The identification of evolutionarily conserved states makes it possible to use the mouse as a model system for this, through lineage tracing and depletion experiments. Notably, using such an approach, we recently demonstrated that a population of MES cells, present in primary tumors in minute amounts, drives the metastatic process (Karras et al. 2022, Nature 610:190-198).

Importantly, our single-cell RNA sequencing data made it possible to identify true malignant MES cells and to develop a set of markers that unambiguously distinguish MES cells from CAFs in scRNA-seq datasets, as well as in situ. This is a critical step forward for the field since the contribution of these cells to various aspects of melanoma biology has remained difficult to establish due to the lack of such a method. In addition, the data we provide herein may also be used to infer the proportion of MES cells from bulk transcriptomics data.

Although previous studies indicated that melanoma de-differentiation may contribute to immune escape (Landsberg et al. 2012, Nature 490:412-416; Mehta et al. 2018, Cancer Discov 8:935-943), a clear association between melanoma MES and resistance to immune checkpoint inhibition has not been formally established. Our data provide evidence that these cells may contribute to primary resistance to ICB and that their presence after one cycle of ICI therapy is predictive of lack of response. This observation indicates that (multiplex) analysis of an early on-treatment biopsy (2 weeks after the first infusion of immune checkpoint inhibitors) may provide a predictive biomarker for robust stratification of patients into Responders (R) and Non-Responders (NR) and thus before NR patients develop any significant adverse events. This observation validates the design of our clinical study, and our initial hypothesis that early on-treatment samples may be much more informative than baseline samples. Although the presence of MES in the on-treatment samples is predictive of lack of response, their proportion remains overall relatively low (below 20% of all malignant cells for most samples) at this early time point. Additional studies will clarify whether their proportion increases at later time points. One interesting possibility is that the MES population may also contribute to primary resistance to ICB in a non-cell autonomous manner, by promoting an immunosuppressive environment. In support of this possibility, emerging data indicates that cells harbouring overlapping phenotypes with melanoma MES cells, such as inflammatory fibroblasts and mesenchymal carcinoma cells, do secrete immunosuppressive factors such as CD73 (Magagna et al. 2021, Cancers 13:5878). We identified TCF4 as a key driver of the mesenchymal-like transcriptional program. Other TFs such as c-JUN/AP1 (Cano & Portillo 2010, Cell Adhes Migr 4:56-60), ZEB1 (Forrest et al. 2013, PLOS One 8: e73169), SOX9 (Jain et al. 2019, Sci Transl Med 11: eaav5599), TEADs (Karras et al. 2022, Nature 610:190-198) and more recently PRRX1 (Karras et al. 2022, Nature 610:190-198) have previously been identified as master regulators of this particular program. It will be interesting to study how redundant and interconnected the activity of these TFs are.

Critically, our data suggest that, in addition to driving the mesenchymal-like transcriptional program and related phenotypes, TCF4 actively suppresses both the melanocytic and antigen presentation programs. By doing so, TCF4 may directly promote immune cell evasion and resistance to immunotherapy. Indeed, melanocytic antigens are prime targets of the adaptive immune system. Moreover, by suppressing the antigen processing and presentation machinery, TCF4 may further reduce the immunogenicity of this de-differentiated melanoma subpopulation. Together, these data identify TCF4 as a putative target to improve response to ICB.

One potential limitation of targeting TCF4 is that this TF is also expressed in other cell types. However, beside melanoma MES cells, TCF4 expression is the highest in DCs, where it was shown to act as a major suppressor of their immunogenic function (Manoharan et al. 2021, J Immunol 207:1428-1436). Therefore, manipulation of the TCF4 pathway in DCs could represent a therapeutic opportunity to further boost antitumor immunity.

Given the fact that TCF4 expression is highly dependent on BRD4 function, the use of BET protein inhibitors may offer an alternative strategy to target this pathway. The treatment of cancer with BET-inhibitors has been explored in numerous early clinical trials (Bechter & Schöffski 2020, Pharmacol Ther 208:107479). Toxicity profiles of several generations of inhibitors showed that these agents can be given safely to patients. Unfortunately, these inhibitors have not yet been broadly used in the clinic due to their modest anti-tumor activity when used as single agents. We argue that BET-inhibitors remain, however, attractive drugs for combinatorial treatments and when used in the appropriate clinical settings.

Our observation that BET-inhibition sensitizes melanoma cells to BRAF- and MEK-inhibition offer one clinical context in which BET-inhibitors may provide clinical benefit.

Another attractive clinical context in which BETi could be positioned is in combination with ICB. We provide evidence that exposure of melanoma MES cells to the BET-inhibitor ARV-711, just like TCF4 silencing, unleashes the expression of antigen presentation machinery and HLA-genes. These data therefore offer a rationale to increase the immunogenicity of melanoma MES cells and warrant the further testing of BET-inhibition in combination with ICB to overcome primary resistance. Notably, recent preclinical studies support this possibility (Echevarría-Vargas et al. 2018, EMBO Mol Med 10: e8446: Nikbakht et al. 2019, J Invest Dermatol 139:1612-1615; Erkes et al. 2019, Pigment Cell Melanoma Res 32:687-696).

Moreover, since emergence of the mesenchymal-like signature was shown to be prominent in patients who experience disease progression after first line immunotherapy (Lee et al. 2020, Nat Commun 11:1897), one could envision that BET-inhibition could reinvigorate anti-tumor immune responses and overcome secondary resistance to ICB.

Lower efficacy was observed with ICB when given as second-line treatment, after first line targeted therapy (Atkins et al. 2021, J Clin Oncol 3:356154-356154). It has recently been proposed that this cross-resistance phenomenon may be driven, at least partly, by changes in the TIME induced by BRAF and MEK-inhibition, leading to a lack of functional CD103+ DCs, and consequently an ineffective T cell response (Haas et al. 2021, Nat cancer 2:693-708). Our findings may offer an alternative (but not mutually exclusive) explanation, invoking a cancer-cell intrinsic mechanism. It is well-established that melanoma MES cells are key drivers of tolerance and/or resistance to targeted therapy. Likewise, we show that this population is enriched in (early on-treatment) lesions from non-responders to ICB, and therefore propose that MES cells may drive, at least partly, cross-resistance to these treatments. Importantly, we show that these cells are exquisitely sensitive to the BRAF/MEK/BET-i triple combination. This combination may therefore also offer an attractive treatment strategy for patients who fail to respond to immunotherapy.

Together, our data offer the rationale for the (pre-) clinical testing of BET-inhibition as both a putative sensitizer to targeted therapy and ICB and for the treatment of patients that develop secondary resistance to these therapies. We argue, however, that the testing of these new combination treatment regimens should be accompanied by a careful selection of the models and/or patients. In this context, the method we describe herein, which allows the unambiguous identification of melanoma MES cells in tumor biopsies, should be considered as a putative biomarker.

1. An inhibitor of transcription factor 4 (TCF4/ITF2) for use in treating or inhibiting cancer, or for use in inhibiting progression, relapse or metastasis of cancer, wherein the cancer is poorly responding to or resistant to immune checkpoint inhibitor therapy or to therapy comprising an immune checkpoint inhibitor.

2. An inhibitor of transcription factor 4 (TCF4/ITF2) for use in treating or inhibiting cancer, or for use in inhibiting progression, relapse or metastasis of cancer,
wherein the patient having cancer has received at least one administration of an immune checkpoint inhibitor therapy or of a therapy comprising an immune checkpoint inhibitor; and
wherein, after the at least one administration of an immune checkpoint inhibitor therapy or of a therapy comprising an immune checkpoint inhibitor the cancer is expressing levels of TCF4/ITF2 corresponding to TCF4/ITF2 expression levels in the same type of cancer of patients known not to respond to the immune checkpoint inhibitor therapy or to the therapy comprising an immune checkpoint inhibitor; and/or
wherein the patient having cancer has received at least one administration of an immune checkpoint inhibitor therapy or of a therapy comprising an immune checkpoint inhibitor; and
wherein, after the at least one administration of an immune checkpoint inhibitor therapy or of a therapy comprising an immune checkpoint inhibitor the cancer is comprising a number of mesenchymal-like cells corresponding to numbers of mesenchymal-like cells in the same type of cancer of patients known not to respond to the immune checkpoint inhibitor therapy or to the therapy comprising an immune checkpoint inhibitor.

3. The inhibitor of TCF4/ITF2 for use according to claim 1 or 2 in combination with immune checkpoint inhibitor therapy or with a therapy comprising an immune checkpoint inhibitor.

4. The inhibitor of TCF4/ITF2 for use according to any of the foregoing claims wherein the inhibitor of TCF4/ITF2 is a specific inhibitor of TCF4/ITF2, and is selected from a DNA nuclease specifically knocking out or disrupting TCF4/ITF2, an RNase specifically targeting TCF4/ITF2, or an inhibitory oligonucleotide specifically targeting TCF4/ITF2.

5. An immune checkpoint inhibitor for use in treating or inhibiting cancer, or for use in inhibiting progression, relapse or metastasis of cancer, in combination with an inhibitor of transcription factor 4 (TCF4/ITF2).

6. The immune checkpoint inhibitor for use according to claim 5, wherein the inhibitor of TCF4/ITF2 is a specific inhibitor of TCF4/ITF2.

7. A combination of an inhibitor of transcription factor 4 (TCF4/ITF2) and an immune checkpoint inhibitor.

8. A composition comprising the combination according to claim 7.

9. The combination according to claim 7 or the composition according to claim 8 for use as a medicine.

10. A method for determining or predicting response of a cancer patient to immune checkpoint inhibitor therapy or to therapy comprising an immune checkpoint inhibitor, comprising:
determining, assessing, measuring, or quantifying the level of TCF4/ITF2 expression in a sample obtained from a cancer lesion, wherein the sample is obtained after the patient having cancer received at least one administration of an immune checkpoint inhibitor therapy or of a therapy comprising an immune checkpoint inhibitor;
determining or predicting the cancer patient having cancer to respond to the immune checkpoint inhibitor therapy or therapy comprising the immune checkpoint inhibitor when the TCF4/ITF2 expression level in the sample corresponds to TCF4/ITF2 expression levels in the same type of cancer of patients known to respond to the immune checkpoint inhibitor therapy or to the therapy comprising an immune checkpoint inhibitor.

11. A method for determining or predicting response of a cancer patient to immune checkpoint inhibitor therapy or to therapy comprising an immune checkpoint inhibitor, comprising:
determining, assessing, measuring, or quantifying the number of mesenchymal-like cancer cells in a sample obtained from a cancer lesion, wherein the sample is obtained after the patient having cancer received at least one administration of an immune checkpoint inhibitor therapy or of a therapy comprising an immune checkpoint inhibitor;
determining or predicting the cancer patient having cancer to respond to the immune checkpoint inhibitor therapy or therapy comprising the immune checkpoint inhibitor when the number of mesenchymal-like cancer cells in the sample corresponds to the number of mesenchymal-like cancer cells in the same type of cancer of patients known to respond to the immune checkpoint inhibitor therapy or to the therapy comprising an immune checkpoint inhibitor.

12. The method according to claim 10 or 11 further comprising:
determining, assessing, measuring, or quantifying the number of antigen-presenting cancer cells in a sample obtained from a cancer lesion, wherein the sample is obtained before the patient having cancer received at least one administration of an immune checkpoint inhibitor therapy or of a therapy comprising an immune checkpoint inhibitor;
determining or predicting the cancer patient having cancer to respond to the immune checkpoint inhibitor therapy or therapy comprising the an immune checkpoint inhibitor when the number of antigen-presenting cancer cells in the sample corresponds to the number of antigen-presenting cancer cells in the same type of cancer of patients known to respond to the immune checkpoint inhibitor therapy or to the therapy comprising an immune checkpoint inhibitor.

13. A method of determining the presence of mesenchymal-like cancer cells in a tumor, comprising:
assessing the presence of a protein and/or RNA encoding the protein in a sample obtained from the tumor, wherein the protein and/or RNA is a product of a gene of Table 1 or 3;
assessing the presence of a protein and/or RNA encoding the protein in a sample obtained from the tumor, wherein the protein and/or RNA is a product of a gene of Table 4 or 5;
assessing the presence of a protein and/or RNA encoding the protein in a sample obtained from the tumor, wherein the protein and/or RNA is a product of a cancer cell gene;
determining mesenchymal-like cancer cells to be present in a tumor when detecting cancer cells in which a product of a gene of Table 1 or 3, a product of a gene of Table 4 or 5, and a product of a cancer cell gene are present.

14. A method of determining the presence of mesenchymal-like cancer cells in a tumor, comprising detecting co-localization of a product of a gene of Table 1 or 3, a product of a gene of Table 4 or 5, and a product of a cancer cell gene in the same cancer cell in a sample obtained from a patient having cancer, and designating such cancer cell as mesenchymal-like cancer cell.

15. The method according to claim 13 or 14 wherein the gene of Table 1 or 3 is THY1, LUM, DCN, and/or TCF; the gene of Table 4 or 5 is CDH19, S100A1, MITF and/or SOX10; and the cancer cell gene is MITF and/or SOX10, and/or is gene with a mutation specific to the cancer cell.


SEQUENCE LISTING (5 sequences)

SEQ ID NO: 1 - protein, 9 aa, synthetic construct
SLLMWITQC

SEQ ID NO: 2 - DNA, 345 bp, synthetic construct
  source 1..345: mol_type = other DNA
aagcaagaagtgacacagatccctgccgctctgtctgtgcctgagggcgaaaacctggtgctgaactgcagcttcaccgacagcgccatctacaacctgcagtggttcagacaggaccccggcaagggactgacaagcctgctgctgattcagagcagccagagagagcagaccagcggcagactgaatgccagcctggataagtcctccggcagaagcaccctgtatatcgccgcttctcagcctggcgatagcgccacatatctgtgtgccgtgcgacctctgtacggcggcagctacatccctacatttggcagaggcaccagcctgatcgtgcacccctac

SEQ ID NO: 3 - protein, 115 aa, synthetic construct
KQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPY

SEQ ID NO: 4 - DNA, 333 bp, synthetic construct
  source 1..333: mol_type = other DNA
ggcgtgacccagacacctaagttccaggtgctgaaaaccggccagagcatgaccctgcagtgcgcccaggatatgaaccacgagtacatgagctggtacagacaggaccctggcatgggcctgagactgatccactattctgtcggagccggcatcaccgaccagggcgaagttcctaatggctacaacgtgtccagaagcaccaccgaggacttcccactgagactgctgtctgccgctcctagccagaccagcgtgtacttttgtgccagcagctacgtgggcaacaccggcgagctgttttttggcgagggcagcagactgaccgtgctc

SEQ ID NO: 5 - protein, 111 aa, synthetic construct
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVL